(19) United States
(12) Patent Application Publication
(10) Pub. No.: US 2020/0318079 A1
(43) Pub. Date: Oct. 8, 2020

(54) ACTIVITY-DEPENDENT EXPRESSION CONSTRUCTS AND METHODS OF USING THE SAME

(71) Applicant: The Board of Trustees of the Leland Stanford Junior University, Stanford, CA (US)

(72) Inventors: Karl A. Deisseroth, Stanford, CA (US); Li Ye, Palo Alto, CA (US); Charu Ramakrishnan, San Jose, CA (US); Kimberly R. Thompson, Palo Alto, CA (US)

(21) Appl. No.: 16/303,903

(22) PCT Filed: May 23, 2017
(86) PCT No.: PCT/US17/34032

§ 371 Date: Nov. 21, 2018

Related U.S. Application Data
(60) Provisional application No. 62/341,516, filed on May 25, 2016.

(51) Int. Cl. C12N 7/00; C12N 15/86; A61K 48/00; A61K 49/00

(52) U.S. Cl. CPC C12N 7/00; C12N 15/86; A61K 48/005; C12N 2830/002; C12N 2750/14143; C12N 2750/14121; A61K 49/00

(57) ABSTRACT

The present disclosure provides nucleic acid activity-dependent expression vectors and activity-dependent expression cassettes for the activity-dependent expression of an encoded polypeptide. Also provided are recombinant adenoassociated viruses (AAV) containing an expression vector comprising an activity-dependent expression cassette for the activity-dependent expression of an encoded polypeptide by cells infected with the AAV vector. The present disclosure also provides methods for the activity-dependent labeling of cells in vitro or in vivo by introducing into the cells an expression vector containing an activity-dependent regulatory sequence driving expression of a labeling polypeptide. Also provided are methods for the activity-dependent control of cells in vitro or in vivo by introducing into the cells an expression vector containing an activity-dependent regulatory sequence driving expression of a light-responsive polypeptide.

### CROSS-REFERENCE

This application claims the benefit of U.S. Provisional Patent Application No. 62/341,516, filed May 25, 2016, which application is incorporated herein by reference in its entirety.

### INCORPORATION BY REFERENCE OF SEQUENCE LISTING PROVIDED AS A TEXT FILE

A Sequence Listing is provided herewith as a text file, “STAN-1319PRV_SeqList_ST25.txt” created on May 25, 2016 and having a size of 167 KB. The contents of the text file are incorporated by reference herein in their entirety.

### BACKGROUND

Activity-based changes are complex biological processes, both on the cellular and organismal levels, requiring the sensing and conversion of external stimuli into changes in cell function and/or cell behavior. One example of the complexity of cell activity modulation within an organism that is being intensely investigated is the mammalian brain.

The many individual regions and layers of the prefrontal cortex are known to contain cells with a rich diversity of activity patterns. Indeed, otherwise-indistinguishable populations of principal cells exhibiting profoundly distinct changes in activity in response to the same task or stimulus have been characterized by electrophysiological recording and cellular-resolution fluorescence Ca²⁺ imaging. At the same time, datastreams of anatomical and molecular information on prefrontal cell typology have emerged from a variety of methods, also pointing toward rich cellular diversity of principal excitatory neurons despite the traditional view that these cells were more homogenous in nature than the highly diverse and readily separable interneurons. Together these findings have highlighted the morphological, wiring, and electrophysiological diversity of principal neurons even within individual layers and subregions. This diversity is mirrored in the complexity of their activation patterns.

The elevated expression of c-fos is concomitant with many forms of cell activation, where the term “cell activation” can be generally considered as an early phase of biological processes which have in common a long-term phenotypic change, e.g., stimulation of quiescent cells to enter the cell cycle, induction of differentiation and long lasting modification of the functional activity of terminally differentiated cells like macrophages or neurons. The elevated expression of c-fos in neurons has been observed in many instances of neuronal activation both in vitro and in vivo. FOS genes encode leucine zipper proteins that can dimerize with proteins of the JUN family, thereby forming the transcription factor complex AP-1. As such, the FOS proteins have been implicated as regulators of cell proliferation, differentiation, transformation and apoptotic cell death, which functions are induced in response to cell activating events.

Coupling expression of desired proteins to cell activity allows for the visualization of complex cell activity patterns and the modulation of cell responses and behaviors following exposure to particular stimuli.

### SUMMARY

The present disclosure provides nucleic acid activity-dependent expression vectors and activity-dependent expression cassettes for the activity-dependent expression of an encoded polypeptide. Also provided are recombinant adeno-associated viruses (AAV) containing an expression vector comprising an activity-dependent expression cassette for the activity-dependent expression of an encoded polypeptide by cells infected with the AAV vector. The present disclosure also provides methods for the activity-dependent labeling of cells in vitro or in vivo by introducing into the cells an expression vector containing an activity-dependent regulatory sequence driving expression of a labeling polypeptide. Also provided are methods for the activity-dependent control of cells in vitro or in vivo by introducing into the cells an expression vector containing an activity-dependent regulatory sequence driving expression of a light-responsive polypeptide.

The present disclosure provides an expression vector comprising, an activity-dependent expression cassette comprising: (a) a regulatory sequence comprising a c-Fos 5′-non-coding region and a c-Fos first intron sequence; and (b) a polypeptide coding sequence operably linked to the regulatory sequence, wherein the polypeptide encoded by the polypeptide coding sequence is expressed from the expression cassette upon activity-dependent activation of the regulatory sequence. In some cases the vector is a viral vector, including e.g., a recombinant adeno-associated virus (AAV) vector. In some cases, the regulatory sequence is a mammalian c-fos regulatory sequence comprising a mammalian c-Fos 5′-non-coding region and a mammalian c-Fos first intron sequence. In some cases, a mammalian c-fos regulatory sequence is a rodent c-fos regulatory sequence comprising a rodent c-Fos 5′-non-coding region and a rodent c-Fos first intron sequence. In some cases, a rodent c-fos regulatory sequence is a mouse c-fos regulatory sequence comprising a mouse c-Fos 5′-non-coding region and a mouse c-Fos first intron sequence. In some cases, the expression cassette further comprises a sequence encoding a PEST peptide operably linked to the 3′ end of the polypeptide coding sequence. In some cases, the polypeptide coding sequence is heterologous to the c-fos regulatory sequence. In some cases, the polypeptide coding sequence encodes a light-responsive polypeptide. In some cases, a light-responsive polypeptide is a depolarizing opsin or a hyperpolarizing opsin. In some cases, the polypeptide coding sequence encodes a molecular tag. In some cases, the polypeptide coding sequence encodes a calcium sensor or voltage sensor or ion channel. In some cases, the polypeptide coding sequence encodes a toxic protein. In some cases, the polypeptide coding sequence encodes a receptor. In some cases, the polypeptide coding sequence encodes a nuclease. In some cases, the polypeptide coding sequence encodes a transcription factor. In some cases, the polypeptide coding sequence encodes a fusion protein comprising two or more polypeptides selected from the group consisting of: a light-responsive polypeptide, a molecular tag, a calcium sensor or voltage sensor or ion channel, a toxic protein, a receptor, a nuclease and a transcription factor. In some cases, the c-Fos 5′-non-coding region is less than 800 nucleotides in length. In some cases, the c-Fos 5′-non-coding region has a sequence identity of 80% or greater with SEQ ID NO:1. In some cases, the c-Fos first intron sequence comprises the entire first intron of a c-Fos gene or a degenerate sequence thereof. In some cases, the c-Fos first intron has a sequence identity of 80% or greater with SEQ ID NO:2. In some cases, the expression cassette further comprises a sequence of 50 to 200 nucleotides in length positioned between the c-Fos 5′-non-coding region and the c-Fos first intron sequence. In some cases, the sequence of 50 to 200 nucleotides in length comprises a sequence encoding the first exon of a c-Fos gene or a portion thereof. In some cases, the sequence encoding the first exon of a c-Fos gene has a sequence identity of 80% or greater with SEQ ID NO:3.

The present disclosure also provides a recombinant adeno-associated virus (AAV), comprising an expression vector including or excluding, alone or in combination, any of the elements discussed above.

The present disclosure also provides a method for activity-dependent labeling of an active cell, the method comprising: (a) contacting a cell with an expression vector comprising an expression cassette comprising: (i) a regulatory sequence comprising a c-Fos 5′-non-coding region and a c-Fos first intron sequence; and (ii) a coding sequence encoding a labeling polypeptide operably linked to the regulatory sequence; and (b) maintaining the cell under conditions permissive for activity-dependent activation of the regulatory sequence, wherein upon activity-dependent activation of the regulatory sequence the labeling polypeptide is expressed thereby labeling the active cell. In some cases, the contacting is performed in vitro. In some cases, the contacting is performed in vivo. In some cases, the cell is a neuron. In some cases, the neuron is a mammalian neuron. In some cases, the neuron is present in the central nervous system of a vertebrate. In some cases, during the maintaining, the cell is contacted with a stimulus thereby activating the regulatory sequence. In some cases, the stimulus is an electrical stimulus. In some cases, the stimulus is a pharmacological stimulus. In some cases, the contacting is performed in vivo by administering the expression vector to the central nervous system of a vertebrate and the maintaining comprises subjecting the vertebrate to a behavioral task sufficient to activate the regulatory sequence. In some cases, the labeling polypeptide is a molecular tag. In some cases, the labeling polypeptide is a recombinase and the cell comprises a recombination sequence that, upon recombination, induces expression of a molecular tag.

The present disclosure also provides a method for activity-dependent control of an activated cell, the method comprising: (a) contacting a cell with an expression vector comprising an expression cassette comprising: (i) a regulatory sequence comprising a c-Fos 5′-non-coding region and a c-Fos first intron sequence; and (ii) a coding sequence encoding a light-responsive polypeptide operably linked to the regulatory sequence; (b) maintaining the cell under conditions permissive for activity-dependent activation of the regulatory sequence, wherein upon activity-dependent activation of the regulatory sequence the light-responsive polypeptide is expressed in the activated cell; and (c) exposing the activated cell to light sufficient to trigger the light-responsive polypeptide to induce a response in the cell thereby controlling the activated cell. In some cases, the contacting is performed in vitro. In some cases, the contacting is performed in vivo. In some cases, the cell is a neuron. In some cases, the neuron is a mammalian neuron. In some cases, the neuron is present in the central nervous system of a vertebrate. In some cases, during the maintaining, the cell is contacted with a stimulus thereby activating the regulatory sequence. In some cases, the stimulus is an electrical stimulus. In some cases, the stimulus is a pharmacological stimulus. In some cases, the contacting is performed in vivo by administering the expression vector to the central nervous system of a vertebrate and the maintaining comprises subjecting the vertebrate to a behavioral task sufficient to activate the regulatory sequence. In some cases, the response is depolarization. In some cases, the response is hyperpolarization.

### BRIEF DESCRIPTION OF THE FIGURES

FIG. 1A-1H: Images demonstrating brain-wide origin/target-defined project mapping.

FIG. 2A-2K: Additional images demonstrating brain-wide origin/target-defined project mapping.

FIG. 3A-3F: Schematic showing the strategy of expression cassette construction, and data showing that cocaine and shock-activated mPFC populations have distinct projection targets.

FIG. 4A-4F: Additional data showing that cocaine and shock-activated mPFC populations have distinct projection targets.

FIG. 5A-5G: Data showing the use of fosCh for targeting cocaine and shock-activated mPFC populations.

FIG. 6A-6B: Additional data showing the use of fosCh for targeting cocaine and shock-activated mPFC populations.

FIG. 7A-7E: Schematic showing the placement of electrodes for recording experiments, and data showing the differential behavioral influence of cocaine and shock-activated mPFC populations.

FIG. 8A-8B: Additional data showing the differential behavioral influence of cocaine and shock-activated mPFC populations.

FIG. 9 provides the sequence of a mouse c-Fos-5′-non-coding region, c-Fos first exon and c-Fos first intron regulatory region.

FIG. 10 provides the sequence of an alternative mouse c-Fos regulatory region.

FIG. 11 provides the sequence of an alternative mouse c-Fos regulatory region.

FIG. 12 provides a map of vector pAAV-cFos-DIO-eNpHR 3.0-eYFP-PEST.

FIG. 13 provides a map of vector pAAV-cFos-DIO-hChR2(H134R)-eYFP-PEST.

FIG. 14 provides a map of vector pAAV-cFos-ER-CreT-ER-ds-p2A.

FIG. 15 provides a map of vector pAAV-cFos-eYFP-PEST.

FIG. 16 provides a map of vector pAAV-cFos-hChR2(H134R)-eYFP-PEST.

FIG. 17 provides a map of vector pAAV-cFos-WGA-Cre.

FIG. 18 provides a map of vector pAAV-cFos-WGA-Cre-WPRE.

FIG. 19 provides the sequences of useful light-responsive polypeptides as described herein (SEQ ID NOs:30-64).

### DEFINITIONS

The term “promoter” as used herein refers to a regulatory region of genomic or recombinant nucleic acid that is composed of one or more transcription start sites and generally contains binding sites for transcription factors and/or transcription factor complexes of the basal transcription machinery.

The term “enhancer” as used herein refers to a cis-acting sequence that increases the utilization of one or more neighboring eukaryotic promoters. Enhancers and can function in either orientation (i.e., “forward” or “reverse”) and in any location (3′, i.e., “downstream”, or 5′, i.e., “upstream”) relative to the promoter.

The term “5′-non-coding region” as used herein refers to non-coding nucleic acid sequence (i.e., nucleic acid sequence that does not code for a naturally produced polypeptide) present adjacent to and 5′ or “upstream” of the start codon (i.e., first translated codon in the protein derived from a protein coding gene) of a gene and generally containing one or more regulatory elements that modulate gene expression. Thus, by “5′-non-coding region promoter” is meant a promoter present within the nucleic acid sequence upstream of the start codon of a gene. Accordingly, as used herein, the 5′-non-coding region may include but is not limited to all or a portion of the genomic sequence that is transcribed into the 5′-untranslated region (5′-UTR) of an RNA expressed from a gene. General features of a 5′-non-coding region include the transcription start site (TSS) of the gene, promoters, enhancers, etc. However, depending on the length of sequence extracted from the 5′ non-coding sequence, a 5′-non-coding sequence derived from a gene locus may include or exclude any or all of the above described individual features. In some instances, extracted 5′ non-coding sequence may include nucleic acid sequence upstream of one or more promoters present within the 5′-non-coding region and/or upstream of the TSS.

The term “exon” generally refers to a region of the transcript sequence within a gene which is not removed from the primary RNA transcript by RNA splicing. However, as used herein, in some instances an exon may also refer to a portion of a nucleic acid sequence that encodes all, e.g., in the case of single exon genes, or a portion, e.g., in the case of multi-exon genes, of a protein. Accordingly, in some instances that will be readily apparent, a reference to an exon will exclude a non-coding portion of a transcript, e.g., that is upstream of the translation start site (i.e., start codon) including e.g., the 5′-UTR.

The term “intron” as used herein refers to a region of a primary transcript that is transcribed, but removed from within the transcript by splicing together the sequences, i.e. the exons, on either side of it.

The term “vector” as used herein refers to generally refers to a replicon that has been modified to act as a vector for foreign sequence. An “expression vector” generally refers to a vector that has been modified for the purpose of expressing a coding sequence from the vector. For example, a vector may comprise a coding sequence capable of being expressed in a target cell. As used herein, “vector construct,” “expression vector,” and “gene transfer vector,” generally refer to any nucleic acid construct capable of directing the expression of a gene of interest and which is useful in transferring the gene of interest into target cells. Thus, the term includes cloning and expression vehicles, as well as integrating vectors and non-integrating vectors. Vectors are thus capable of transferring nucleic acid sequences to target cells and, in some instances, are used to manipulate nucleic acid sequence, e.g., recombine nucleic acid sequences (i.e. to make recombinant nucleic acid sequences) and the like. For purposes of this disclosure examples of vectors include, but are not limited to, plasmids, phage, transposons, cosmids, virus, and the like.

The term “recombinant”, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide, means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polypeptide” and “protein” are used interchangeably to refer to a polymer of amino acid residues linked by peptide bonds, and for the purposes of the instant disclosure, have a minimum length of at least 10 amino acids. Oligopeptides, oligomers multimers, and the like, typically refer to longer chains of amino acids and are also composed of linearly arranged amino acids linked by peptide bonds, whether produced biologically, recombinantly, or synthetically and whether composed of naturally occurring or non-naturally occurring amino acids, are included within this definition. Both full-length proteins and fragments thereof greater than 10 amino acids are encompassed by the definition. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

### DETAILED DESCRIPTION

The present disclosure provides nucleic acid activity-dependent expression vectors and activity-dependent expression cassettes for the activity-dependent expression of an encoded polypeptide. Also provided are recombinant adeno-associated viruses (AAV) containing an expression vector comprising an activity-dependent expression cassette for the activity-dependent expression of an encoded polypeptide by cells infected with the AAV vector. The present disclosure also provides methods for the activity-dependent labeling of cells in vitro or in vivo by introducing into the cells an expression vector containing an activity-dependent regulatory sequence driving expression of a labeling polypeptide. Also provided are methods for the activity-dependent control of cells in vitro or in vivo by introducing into the cells an expression vector containing an activity-dependent regulatory sequence driving expression of a light-responsive polypeptide.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Expression Constructs

The present disclosure provides expression constructs for the activity-dependent expression of encoded polypeptides. Expression constructs of the present disclosure will generally include at least a regulatory sequence and a sequence encoding a polypeptide of interest, herein commonly referred to as an “encoded polypeptide”. Polypeptides encoded from the coding sequence of an expression construct, discussed in more detail below, will vary depending in part on the particular goal or end-use of the expression construct.

The elements of the expression constructs of the instant disclosure will generally be arranged with the regulatory sequences “upstream” or 5′ to the polypeptide coding sequence such that the regulatory sequences are operably linked to the coding sequence, meaning the regulatory region and the coding sequence are in such relative orientation that activation of the regulatory region drives expression of the coding sequence(s). The expression constructs of the instant disclosure may further include or exclude, depending on the particular application, particular elements necessary for maintenance, propagation and/or use of the expression construct in a vector, as described in more detail below.

### Regulatory Sequences

Activity-dependent regulatory sequences of the present disclosure contain nucleic acid expression control elements that are responsive to transcription factors that induce expression of the proto-oncogene c-Fos (also known as, depending on the relevant species, FOS, Fos proto-oncogene, AP-1 transcription factor subunit, FBJ osteosarcoma oncogene, and the like). c-Fos immediate and early upregulation is commonly associated with cellular activation including the activation of cells in response to external stimuli. Accordingly, without being bound by theory, it was determined that regulatory elements of c-Fos provide efficient components for the activity-dependent induction of downstream coding sequences.

Regulatory sequences of the herein described expression constructs may include a 5′-non-coding regulatory sequence, intronic sequence or a combination thereof. Regulatory sequences however need not be limited only to those sequences that provide a regulatory function as such sequences may, in some instances, include additional sequence that does not contribute a regulatory function. In some instances, regulatory sequences may be modified to exclude one or more certain sequences not having a regulatory function. The regulatory sequences described herein may be entirely non-coding or may include some coding sequence including e.g., where non-coding sequence is present in combination with one or more coding exons or portions thereof.

A regulatory sequence of an expression construct of the instant disclosure may generally contain a 5′-non-coding regulatory sequence of a c-Fos gene. 5′-non-coding regulatory regions will generally include nucleotide sequence upstream of the 5′ start codon, i.e., the first translated codon of the first exon, of the gene. 5′-non-coding sequence will generally contain at least one promoter element and may also contain but need not necessarily include one or more enhancers. Thus, a c-Fos 5′-non-coding regulatory region includes at least one 5′ c-Fos promoter. The 5′-non-coding region may contain but is not limited to the genomic nucleotide sequence that is transcribed into the 5′-untranslated region (5′-UTR) of the c-Fos gene transcript. A c-Fos 5′-non-coding region may include the c-Fos transcription initiation site or transcription start site (TSS) and may further include non-coding sequence upstream of the c-Fos TSS.

As such, the size of the 5′-non-coding regulatory region of an expression construct of the instant disclosure may vary and may include but is not limited to e.g., more or less than 1 kb of sequence upstream from the start codon of a c-Fos gene, including but not limited to e.g., 1 kb or less of the upstream sequence, 950 bp or less of upstream sequence, 900 bp or less of upstream sequence, 850 bp or less of upstream sequence, 800 bp or less of upstream sequence, 790 bp or less of upstream sequence, 780 bp or less of upstream sequence, 770 bp or less of upstream sequence, 760 bp or less of upstream sequence, 750 bp or less of upstream sequence, 740 bp or less of upstream sequence, 730 bp or less of upstream sequence, 720 bp or less of upstream sequence, 710 bp or less of upstream sequence, 700 bp or less of upstream sequence, etc.

The length of a 5′ non-coding regulatory region of an expression construct of the present disclosure may vary and may range from less than 250 bp to 1 kb or more than 1 kb; for example, the length of a 5′ non-coding regulatory region of an expression construct of the present disclosure can range from 250 bp to 900 bp, 250 bp to 850 bp, 250 bp to 800 bp, 250 bp to 750 bp, 250 bp to 700 bp, 250 bp to 650 bp, 250 bp to 600 bp, 250 bp to 550 bp, 250 bp to 500 bp, 500 bp to 900 bp, 500 bp to 850 bp, 500 bp to 800 bp, 500 bp to 750 bp, 500 bp to 700 bp, 500 bp to 650 bp, 500 bp to 600 bp, 750 bp to 900 bp, 750 bp to 850 bp, 750 bp to 800 bp, etc.

A regulatory sequence of an expression construct of the instant disclosure may generally contain sequence of a first intron of a c-Fos gene, whereby “first intron” is meant the non-coding sequence immediately following (i.e., downstream of the 3′ splice site) of the first exon of a c-Fos gene that is spliced out during processing of the c-Fos transcript. Accordingly, by “sequence of a first intron” is meant the genomic sequence corresponding to the spliced out intronic transcript sequence. Expression cassettes may include the entire first intron sequence or a portion of the first intron sequence including but not limited to e.g., a percentage of the full-length first intron including but not limited to e.g., 100%, 99%, 98%, 97%, 96%, 95%, 90%, 85%, 80%, 75%, 70%, 65%, 60%, 55%, 50%, 45%, 40%, 35%, 30%, 25%, 20%, etc., of the first intron. As such, the length of the c-Fos first intron sequence present in an expression construct of the instant disclosure will vary, depending in part on the source of the c-Fos intron (i.e., the c-Fos gene from which the first intron sequence is derived), and may include but are not limited to e.g., 800 bp or less, 795 bp or less, 790 bp or less, 785 bp or less, 780 bp or less, 775 bp or less, 770 bp or less, 765 bp or less, 760 bp or less, 755 bp or less, 754 bp or less, 753 bp or less, 752 bp or less, 751 bp or less, 750 bp or less, 725 bp or less, 700 bp or less, 675 bp or less, 650 bp or less, 625 bp or less, 600 bp or less, 575 bp or less, 550 bp or less, 525 bp or less, 500 bp or less, 475 bp or less, 450 bp or less, 425 bp or less, 400 bp or less, 375 bp or less, 350 bp or less, 325 bp or less, 300 bp or less, 275 bp or less, 250 bp or less, 225 bp or less, 200 bp or less, 175 bp or less, 150 bp or less, 125 bp or less, 100 bp or less, 75 bp or less, 50 bp or less, and the like.

In some instances, the length of a sequence of a c-Fos first intron of an expression construct may range from 25 bp to 1 kb, or more than 1 kb; e.g., the length of a c-Fos first intron of an expression construct of the present disclosure can range from, e.g., 25 bp to 1000 bp, 25 bp to 900 bp, 25 bp to 800 bp, 25 bp to 700 bp, 25 bp to 600 bp, 25 bp to 500 bp, 25 bp to 400 bp, 25 bp to 300 bp, 25 bp to 200 bp, 25 bp to 100 bp, 50 bp to 1000 bp, 50 bp to 900 bp, 50 bp to 800 bp, 50 bp to 700 bp, 50 bp to 600 bp, 50 bp to 500 bp, 50 bp to 400 bp, 50 bp to 300 bp, 50 bp to 200 bp, 50 bp to 100 bp, 100 bp to 1000 bp, 100 bp to 900 bp, 100 bp to 800 bp, 100 bp to 700 bp, 100 bp to 600 bp, 100 bp to 500 bp, 100 bp to 400 bp, 100 bp to 300 bp, 100 bp to 200 bp, 200 bp to 1000 bp, 200 bp to 900 bp, 200 bp to 800 bp, 200 bp to 700 bp, 200 bp to 600 bp, 200 bp to 500 bp, 200 bp to 400 bp, 200 bp to 300 bp, 300 bp to 1000 bp, 300 bp to 900 bp, 300 bp to 800 bp, 300 bp to 700 bp, 300 bp to 600 bp, 300 bp to 500 bp, 300 bp to 400 bp, 500 bp to 1000 bp, 500 bp to 900 bp, 500 bp to 800 bp, 500 bp to 700 bp, 500 bp to 600 bp, etc. In some instances, a c-Fos first intron sequence of an expression construct may start at the first intron 5′ spice site and may continue for a desired length, including e.g., a length as described herein. In some instances, a c-Fos first intron sequence may exclude the 5′ splice site and/or one or more nucleotides 3′ of the 5′ slice site including e.g., 1 to 100 nucleotides adjacent to and 3′ of the 5′ splice site, including but not limited to e.g., 1 to 75 nucleotides, 1 to 50 nucleotides, 1 to 25 nucleotides, 1 to 20 nucleotides, 1 to 15 nucleotides, 1 to 10 nucleotides, 1 to 5 nucleotides, etc. In some instances, a c-Fos first intron sequence may include sequence adjacent to the 5′ splice site and, in some instances, may include the 5′ splice site. In some instances, a c-Fos first intron sequence may exclude sequence adjacent to the 5′ splice site and, in some instances, may exclude the 5′ splice site.

In some instances, a regulatory sequence of an expression construct of the instant disclosure may include all or a portion of one or more exons of a c-Fos gene, including but not limited to e.g., all or a portion of the first exon of a c-Fos gene, all or a portion of the second exon of a c-Fos gene, etc. In some instances, a regulatory sequence that includes sequence upstream and downstream of an exon of a c-Fos gene may be modified to remove all or a portion of the sequence encoding the exon resulting in a regulatory sequence that lacks c-Fos exons or lacks a complete c-Fos exon. For example, in some instances, a c-Fos regulatory sequence may include c-Fos 5′-non-coding sequence and c-Fos first intron sequence but exclude all or a portion of the c-Fos first exon. In some instances, a c-Fos regulatory sequence may include c-Fos 5′-non-coding sequence and c-Fos first intron sequence and all or a portion of the c-Fos first exon.

As described herein, the regulatory elements, and sequences accompanying or adjacent to such regulatory elements, including e.g., exons, of the expression constructs of the instant disclosure may be derived from one or more c-Fos genes. Useful c-Fos genes for deriving regulatory elements as described herein include c-Fos genes isolated or cloned from, in whole or in part, or identified in an individual, examples of which include but are not limited to e.g., invertebrate c-Fos genes, vertebrate c-Fos genes, mammalian c-Fos genes, rodent c-Fos genes, primate c-Fos genes, lagomorph c-Fos genes, canine c-Fos genes, feline c-Fos genes, ungulate c-Fos genes, primate c-Fos genes, non-human primate c-Fos genes, human c-Fos genes, etc.

Useful c-Fos genes include but are not limited to e.g., NCBI GeneID 14281 from Mus musculus present on chromosome 12 map location 12 39.7 cM (RefSeq NC_000078.6), NCBI GeneID 314322 from Rattus norvegicus present on chromosome 6 map location 6q31 (RefSeq NC_005105.4), NCBI GeneID 2353 from Homo sapiens present on chromosome 14 map location 14q24.3 (RefSeq NC_000014.9), NCBI GeneID 3772082 from Drosophila melanogaster present on chromosome 3R map location 3-99 cM (RefSeq NT_033777.3), NCBI GeneID 493935 from Felis catus present on chromosome B3 map location (RefSeq NC_018728.2), NCBI GeneID 100144486 from Sus scrofa present on chromosome 7 (RefSeq NC_010449.4), NCBI GeneID 702077 from Macaca mulatta present on chromosome 7 (RefSeq NC_027899.1), NCBI GeneID 453047 from Pan troglodytes present on chromosome 14 (RefSeq NC_006481.3), NCBI GeneID 443218 from Ovis aries present on chromosome 7 (RefSeq NC_019464.2), NCBI GeneID 548954 from Xenopus tropicalis, NCBI GeneID 100820712 from Oryzias latipes present on chromosome 24 (RefSeq NC_019882.1), NCBI GeneID 447201 from Xenopus laevis, NCBI GeneID 103457600 from Poecilia reticulata present on chromosome LG21 (RefSeq NC_024351.1), NCBI GeneID 101959407 from Ictidomys tridecemlineatus, NCBI GeneID 101831721 from Mesocricetus auratus, and the like.

For example, in some instances, a c-Fos gene from which regulatory elements may be derived may be a mouse c-Fos gene including e.g., NCBI Gene ID:14281 encoding e.g., RefSeq NP_034364.1 (SEQ ID NO:19) from transcript RefSeq NM_010234.2 (SEQ ID NO:20). Exemplary ′5-non-coding region sequence of a mouse c-Fos gene includes but is not limited to e.g., the 1.5 kb sequence upstream from the start codon provided in SEQ ID NO:4. In some instances, a useful mouse c-Fos 5′-non-coding region will include the following sequence, in whole or in part, which represents 767 bp upstream of the start codon of the mouse c-Fos gene:

(SEQ ID NO: 5)
GTGGGCAAGCTTTCCTTTAGGAACAGAGGCTTCGAGCCTTTAAGGCTGCG
TACTTGCTTCTCCTAATACCAGAGACTCAAAAAAAAAAAAAAAGTTCCAG
ATTGCTGGACAATGACCCGGGTCTCATCCCTTGACCCTGGGAACCGGGTC
CACATTGAATCAGGTGCGAATGTTCGCTCGCCTTCTCTGCCTTTCCCGCC
TCCCCTCCCCCGGCCGCGGCCCCGGTTCCCCCCCTGCGCTGCACCCTCAG
AGTTGGCTGCAGCCGGCGAGCTGTTCCCGTCAATCCCTCCCTCCTTTACA
CAGGATGTCCATATTAGGACATCTGCGTCAGCAGGTTTCCACGGCCGGTC
CCTGTTGTTCTGGGGGGGGGACCATCTCCGAAATCCTACACGCGGAAGGT
CTAGGAGACCCCCTAAGATCCCAAATGTGAACACTCATAGGTGAAAGATG
TATGCCAAGACGGGGGTTGAAAGCCTGGGGCGTAGAGTTGACGACAGAGC
GCCCGCAGAGGGCCTTGGGGCGCGCTTCCCCCCCCTTCCAGTTCCGCCCA
GTGACGTAGGAAGTCCATCCATTCACAGCGCTTCTATAAAGGCGCCAGCT
GAGGCGCCTACTACTCCAACCGCGACTGCAGCGAGCAACTGAGAAGACTG
GATAGAGCCGGCGGTTCCGCGAACGAGCAGTGACCGCGCTCCCACCCAGC
TCTGCTCTGCAGCTCCCACCAGTGTCTACCCCTGGACCCCTTGCCGGGCT
TTCCCCAAACTTCGACC.

In some instances, a c-Fos 5′-non-coding region of an expression construct of the instant disclosure may include a sequence having 100% identity with SEQ ID NO:5. In some instances, a c-Fos 5′-non-coding region of an expression construct of the instant disclosure may include a sequence having less than 100% identity with SEQ ID NO:5, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:5.

In some instances, a useful mouse c-Fos 5′-non-coding region will include the following sequence, in whole or in part, which represents 761 bp upstream of the start codon of the mouse c-Fos gene:

(SEQ ID NO: 1)
AAGCTTTCCTTTAGGAACAGAGGCTTCGAGCCTTTAAGGCTGCGTACTTG
CTTCTCCTAATACCAGAGACTCAAAAAAAAAAAAAAAGTTCCAGATTGCT
GGACAATGACCCGGGTCTCATCCCTTGACCCTGGGAACCGGGTCCACATT
GAATCAGGTGCGAATGTTCGCTCGCCTTCTCTGCCTTTCCCGCCTCCCCT
CCCCCGGCCGCGGCCCCGGTTCCCCCCCTGCGCTGCACCCTCAGAGTTGG
CTGCAGCCGGCGAGCTGTTCCCGTCAATCCCTCCCTCCTTTACACAGGAT
GTCCATATTAGGACATCTGCGTCAGCAGGTTTCCACGGCCGGTCCCTGTT
GTTCTGGGGGGGGGACCATCTCCGAAATCCTACACGCGGAAGGTCTAGGA
GACCCCCTAAGATCCCAAATGTGAACACTCATAGGTGAAAGATGTATGCC
AAGACGGGGGTTGAAAGCCTGGGGCGTAGAGTTGACGACAGAGCGCCCGC
AGAGGGCCTTGGGGCGCGCTTCCCCCCCCTTCCAGTTCCGCCCAGTGACG
TAGGAAGTCCATCCATTCACAGCGCTTCTATAAAGGCGCCAGCTGAGGCG
CCTACTACTCCAACCGCGACTGCAGCGAGCAACTGAGAAGACTGGATAGA
GCCGGCGGTTCCGCGAACGAGCAGTGACCGCGCTCCCACCCAGCTCTGCT
CTGCAGCTCCCACCAGTGTCTACCCCTGGACCCCTTGCCGGGCTTTCCCC
AAACTTCGACC.

In some instances, a c-Fos 5′-non-coding region of an expression construct of the instant disclosure may include a sequence having 100% identity with SEQ ID NO:1. In some instances, a c-Fos 5′-non-coding region of an expression construct of the instant disclosure may include a sequence having less than 100% identity with SEQ ID NO:1, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:1.

In some instances, a useful mouse c-Fos first intron sequence will include, in whole or in part, the following sequence which represents the 754 bp first intron of the mouse c-Fos gene:

(SEQ ID NO: 2)
GTGAGTTTGGCTTTGTGTAGCCGCCAGGTCCGCGCTGAGGGTCGCCGTGG
AGGAGACACTGGGGTGTGACTCGCAGGGGCGGGGGGGTCTTCCTTTTTCG
CTCTGGAGGGAGACTGGCGCGGTCAGAGCAGCCTTAGCCTGGGAACCCAG
GACTTGTCTGAGCGCGTGCACACTTGTCATAGTAAGACTTAGTGACCCCT
TCCCGCGCGGCAGGTTTATTCTGAGTGGCCTGCCTGCATTCTTCTCTCGG
CCGACTTGTTTCTGAGATCAGCCGGGGCCAACAAGTCTCGAGCAAAGAGT
CGCTAACTAGAGTTTGGGAGGCGGCAAACCGCGGCAATCCCCCCTCCCGG
GGCAGCCTGGAGCAGGGAGGAGGGAGGAGGGAGGAGGGTGCTGCGGGCGG
GTGTGTAAGGCAGTTTCATTGATAAAAAGCGAGTTCATTCTGGAGACTCC
GGAGCAGCGCCTGCGTCAGCGCAGACGTCAGGGATATTTATAACAAACCC
CCTTTCGAGCGAGTGATGCCGAAGGGATAACGGGAACGCAGCAGTAGGAT
GGAGGAGAAAGGCTGCGCTGCGGAATTCAAGGGAGGATATTGGGAGAGCT
TTTATCTCCGATGAGGTGCATACAGGAAGACATAAGCAGTCTCTGACCGG
AATGCTTCTCTCTCCCTGCTTCATGCGACACTAGGGCCACTTGCTCCACC
TGTGTCTGGAACCTCCTCGCTCACCTCCGCTTTCCTCTTTTTGTTTTGTT
TCAG.

In some instances, a c-Fos intron sequence of an expression construct of the instant disclosure may include a sequence having 100% identity with SEQ ID NO:2. In some instances, a intron sequence of an expression construct of the instant disclosure may include a sequence having less than 100% identity with SEQ ID NO:2, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:2.

In some instances, a regulatory region may include a mouse c-Fos first exon coding sequence, in whole or in part, including e.g., the following mouse c-Fos first exon coding sequence or a portion thereof:

(SEQ ID NO: 3)
ATGATGTTCTCGGGTTTCAACGCCGACTACGAGGCGTCATCCTCCCGCTG
CAGTAGCGCCTCCCCGGCCGGGGACAGCCTTTCCTACTACCATTCCCCAG
CCGACTCCTTCTCCAGCATGGGCTCTCCTGTCAACACACAG.

In some instances, a c-Fos exon sequence of an expression construct of the instant disclosure may include a sequence having 100% identity with SEQ ID NO:3. In some instances, a exon sequence of an expression construct of the instant disclosure may include a sequence having less than 100% identity with SEQ ID NO:3, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:3.

In some instances, a regulatory region of an expression construct of the instant disclosure may include, consist essentially of or be the regulatory region, containing a mouse 5′-non-coding region, a mouse first exon and a mouse first intron sequence presented in SEQ ID NO: 7.

In some instances, a c-Fos gene from which regulatory elements may be derived may be a human c-Fos gene including e.g., NCBI Gene ID:2353 (NG_029673.1) encoding e.g, RefSeq NP_005243.1 (SEQ ID NO:21) from transcript RefSeq NM_005252.3 (SEQ ID NO:22). Exemplary ′5-non-coding region sequence of a human c-Fos gene includes but is not limited to e.g., the 1.5 kb sequence upstream from the start codon provided in SEQ ID NO:8. In some instances, a useful human c-Fos 5′-non-coding region will include the following sequence, in whole or in part, which represents 784 bp upstream of the start codon of the human c-Fos gene:

(SEQ ID NO: 9)
GTAGGGGCGCATTCCTTCGGGAGCCGAGGCTTAAGTCCTCGGGGTCCTGT
ACTCGATGCCGTTTCTCCTATCTCTGAGCCTCAGAACTGTCTTCAGTTTC
CGTACAAGGGTAAAAAGGCGCTCTCTGCCCCATCCCCCCCGACCTCGGGA
ACAAGGGTCCGCATTGAACCAGGTGCGAATGTTCTCTCTCATTCTGCGCC
GTTCCCGCCTCCCCTCCCCCAGCCGCGGCCCCCGCCTCCCCCCGCACTGC
ACCCTCGGTGTTGGCTGCAGCCCGCGAGCAGTTCCCGTCAATCCCTCCCC
CCTTACACAGGATGTCCATATTAGGACATCTGCGTCAGCAGGTTTCCACG
GCCTTTCCCTGTAGCCCTGGGGGGAGCCATCCCCGAAACCCCTCATCTTG
GGGGGCCCACGAGACCTCTGAGACAGGAACTGCGAAATGCTCACGAGATT
AGGACACGCGCCAAGGCGGGGGCAGGGAGCTGCGAGCGCTGGGGACGCAG
CCGGGCGGCCGCAGAAGCGCCCAGGCCCGCGCGCCACCCCTCTGGCGCCA
CCGTGGTTGAGCCCGTGACGTTTACACTCATTCATAAAACGCTTGTTATA
AAAGCAGTGGCTGCGGCGCCTCGTACTCCAACCGCATCTGCAGCGAGCAT
CTGAGAAGCCAAGACTGAGCCGGCGGCCGCGGCGCAGCGAACGAGCAGTG
ACCGTGCTCCTACCCAGCTCTGCTCCACAGCGCCCACCTGTCTCCGCCCC
TCGGCCCCTCGCCCGGCTTTGCCTAACCGCCACG.

In some instances, a c-Fos 5′-non-coding region of an expression construct of the instant disclosure may include a sequence having 100% identity with SEQ ID NO:9. In some instances, a c-Fos 5′-non-coding region of an expression construct of the instant disclosure may include a sequence having less than 100% identity with SEQ ID NO:9, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:9.

In some instances, a useful human c-Fos first intron sequence will include, in whole or in part, the following sequence which represents the 753 bp first intron of the human c-Fos gene:

(SEQ ID NO: 11)
GTAAGGCTGGCTTCCCGTCGCCGCGGGGCCGGGGGCTTGGGGTCGCGGAG
GAGGAGACACCGGGCGGGACGCTCCAGTAGATGAGTAGGGGGCTCCCTTG
TGCCTGGAGGGAGGCTGCCGTGGCCGGAGCGGTGCCGGCTCGGGGGCTCG
GGACTTGCTCTGAGCGCACGCACGCTTGCCATAGTAAGAATTGGTTCCCC
CTTCGGGAGGCAGGTTCGTTCTGAGCAACCTCTGGTCTGCACTCCAGGAC
GGATCTCTGACATTAGCTGGAGCAGACGTGTCCCAAGCACAAACTCGCTA
ACTAGAGCCTGGCTTCTCCGGGGAGGTGGCAGAAAGCGGCAATCCCCCCT
CCCCCGGCAGCCTGGAGCACGGAGGAGGGATGAGGGAGGAGGGTGCAGCG
GGCGGGTGTGTAAGGCAGTTTCATTGATAAAAAGCGAGTTCATTCTGGAG
ACTCCGGAGCGGCGCCTGCGTCAGCGCAGACGTCAGGGATATTTATAACA
AACCCCCTTTCAAGCAAGTGATGCTGAAGGGATAACGGGAACGCAGCGGC
AGGATGGAAGAGACAGGCACTGCGCTGCGGAATGCCTGGGAGGAAAAGGG
GGAGACCTTTCATCCAGGATGAGGGACATTTAAGATGAAATGTCCGTGGC
AGGATCGTTTCTCTTCACTGCTGCATGCGGCACTGGGAACTCGCCCCACC
TGTGTCCGGAACCTGCTCGCTCACGTCGGCTTTCCCCTTCTGTTTTGTTC
TAG.

In some instances, a c-Fos intron sequence of an expression construct of the instant disclosure may include a sequence having 100% identity with SEQ ID NO:11. In some instances, a intron sequence of an expression construct of the instant disclosure may include a sequence having less than 100% identity with SEQ ID NO:11, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:11.

In some instances, a regulatory region may include a human c-Fos first exon coding sequence, in whole or in part, including e.g., the following human c-Fos first exon coding sequence or a portion thereof:

(SEQ ID NO: 12)
ATGATGTTCTCGGGCTTCAACGCAGACTACGAGGCGTCATCCTCCCGCTG
CAGCAGCGCGTCCCCGGCCGGGGATAGCCTCTCTTACTACCACTCACCCG
CAGACTCCTTCTCCAGCATGGGCTCGCCTGTCAACGCGCAG.

In some instances, a c-Fos exon sequence of an expression construct of the instant disclosure may include a sequence having 100% identity with SEQ ID NO:12. In some instances, a exon sequence of an expression construct of the instant disclosure may include a sequence having less than 100% identity with SEQ ID NO:12, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:12.

In some instances, a regulatory region of an expression construct of the instant disclosure may include, consist essentially of or be the regulatory region, containing a human 5′-non-coding region, a human first exon and a human first intron sequence presented in SEQ ID NO:13.

In some instances, a c-Fos gene from which regulatory elements may be derived may be a rat c-Fos gene including e.g., NCBI Gene ID:314322 encoding e.g, RefSeq NP_071533.1 (SEQ ID NO:23) from transcript RefSeq NM_022197.2 (SEQ ID NO:24). Exemplary ′5-non-coding region sequence of a rat c-Fos gene includes but is not limited to e.g., the 1.5 kb sequence upstream from the start codon provided in SEQ ID NO:14. In some instances, a useful rat c-Fos 5′-non-coding region will include the following sequence, in whole or in part, which represents 770 bp upstream of the start codon of the rat c-Fos gene:

(SEQ ID NO: 15)
GTGGGCTAGCTTTCCTTTGGGAACAGAGACTTGGAGCCTTTAGGGCTGCG
TGCCTGCTTCTCCTAATACCAGAGACTTTTTTAAAAAGCTCCAGATTGCT
GGACAATGGAAAGGAGATGACCCCCAGTCTCATCCCCTGACCCTGGGAAC
AGAGTACACATTGAATCAGGTGCGAATGTTCGCTCGCCTTCTCTGCCTTT
CCCGCCTCCCCTCCCCCGGCCGCGGCCCCCGCTCCCCCCTTGCGCTGCAC
CCTCAGAGTTGGCTGCAGCCGGCGAGCTGTTCCCGTCAATCCCTCCCTCC
TTTACACAGGATGTCCATATTAGGACATCTGCGTCAGCAGGTTTCCACGG
CCGGTCCCTGTTGTCCTGGGGGGAACCATCCCCGAAATCCTACATGCGGA
GGGTCCAGGAGACCTTCTAAGATCCCAATTGTGAACACTCATAGGTGAAA
GTTACAGACTGAGACGGGGGTTGAGAGCCTGGGGCGTAGAGTTGATGACA
GGGAGCCCGCAGAGGGCATTCGGGAGCGCTTTCCCCCCTCCAGTTTCTCT
GTTCCGCTCATGACGTAGTAAGCCATTCAAGCGCTTCTATAAAGCGGCCA
GCTGAGGCGCCTACTACTCCAACCGCGATTGCAGCTAGCAACTGAGAAGA
CTGGATAGAGCCGGCGGAGCCGCGAACGAGCAGTGACCGCGCTCCCACCC
AGCTCTGCTCTGCAGCTCCCACCAGTGTCTACCCCTGGACCCCTCGCCGA
GCTTTGCCCAAACCACGACC.

In some instances, a c-Fos 5′-non-coding region of an expression construct of the instant disclosure may include a sequence having 100% identity with SEQ ID NO:15. In some instances, a c-Fos 5′-non-coding region of an expression construct of the instant disclosure may include a sequence having less than 100% identity with SEQ ID NO:15, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:15.

In some instances, a useful rat c-Fos first intron sequence will include, in whole or in part, the following sequence which represents the 760 bp first intron of the rat c-Fos gene:

(SEQ ID NO: 16)
GGTGAGTTTGGCTTTGTGCAGTCGCCAGGTCCGCGCTGGGGGTCGCCGAG
GAGGGCACATTGGGGTGTGACTGTCAGGGAAGAGTAGGGGTCTTCCTTGT
TTGCTCCGGAGGGAGACTGGCGCGGTCAGAGCAGCCCTAGCCTGGGAACC
CAGGACTTGTCTGAGCGCGTGCACACTTGTCATACTAAGACTTAGTGACC
CCCCTCCCGCGCGGCAGGTTTACTCTGAGTGTCCTGCGCTCTTCTCTCGG
TGACTTGTTTCTGAGATCAGCCGGGGCCAACAAGTCTCTAGCAAAGACTC
GCTAACTAGAGCCTGGGAGGCGGCAAACGGCGGCAATCCCCCCTCCCGGG
GCAGCCTGGAGCAGGGAGAAGGGAGGAGGGAGGAGGGTGCTGCGAGCCGG
TGTGTAAGGCAGTTTCATTGATAAAAAGCGAGTTCATTCTGGAGACTCCG
GAGCAGCGCCTGCGTCAGCGCAGACGTCAGGGATATTTATAACAAACCCC
CTTTCGAGCGAGTGATGCTGAAGGGATAACGGGAACGCAGCAGTAGGATG
GAGGAGAAAGGCTGAGCTGCGGAATTCAGGGGAGGATAGAGGATATTGGG
AGACCTTTTTATCTCGGATGAAGTGCATACAGGAAGACACAAGCAGTCTC
TGACCAGAATGCTTCTCTCTCCCTGCTTCATGCGACACTAGGGCCACTTG
CTCCACCTGTGTCTGGAACCTCCTCGCTCACCTCCGCTTTCCTCTTTTTG
TTTTGTTTCA.

In some instances, a c-Fos intron sequence of an expression construct of the instant disclosure may include a sequence having 100% identity with SEQ ID NO:16. In some instances, a intron sequence of an expression construct of the instant disclosure may include a sequence having less than 100% identity with SEQ ID NO:16, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:16.

In some instances, a regulatory region may include a rat c-Fos first exon coding sequence, in whole or in part, including e.g., the following rat c-Fos first exon coding sequence or a portion thereof:

(SEQ ID NO: 17)
ATGATGTTCTCGGGTTTCAACGCGGACTACGAGGCGTCATCCTCCCGCTG
CAGTAGCGCCTCCCCGGCCGGGGACAGCCTTTCCTACTACCATTCCCCAG
CCGACTCCTTCTCCAGCATGGGCTCCCCTGTCAACACACA.

In some instances, a c-Fos exon sequence of an expression construct of the instant disclosure may include a sequence having 100% identity with SEQ ID NO:17. In some instances, a exon sequence of an expression construct of the instant disclosure may include a sequence having less than 100% identity with SEQ ID NO:17, including but not limited to e.g., a sequence identity of 99% or more, 98% or more, 97% or more, 96% or more, 95% or more, 94% or more, 93% or more, 92% or more, 91% or more, 90% or more, 89% or more, 88% or more, 87% or more, 86% or more, 85% or more, 84% or more, 83% or more, 82% or more, 81% or more, 80% or more, 79% or more, 78% or more, 77% or more, 76% or more, 75% or more, 74% or more, 73% or more, 72% or more, 71% or more, 70% or more, 65% or more, 60% or more, 55% or more, 50% or more, etc., to SEQ ID NO:17.

In some instances, a regulatory region of an expression construct of the instant disclosure may include, consist essentially of or be the regulatory region, containing a rat 5′-non-coding region, a rat first exon and a rat first intron sequence presented in SEQ ID NO:18.

In some instances, a c-Fos regulatory region may include one or more of the following sequences containing putative c-Fos promoters:

(SEQ ID NO: 6; mouse)
tccattcacagcgcttctataaaggcgccagctgaggcgcctactactcC
AACCGCGACT;
(SEQ ID NO: 10; human)
ttcataaaacgcttgttataaaagcagtggctgcggcgcctcgtactccA
ACCGCATCTG.

In constructing a regulatory region within an expression construct of the instant disclosure the described regulatory sequences may be combined or substituted as appropriate. For example, the individual components, or fragments thereof, from a particular species (e.g., mouse, rat, human, etc.) may be combined, in whole or in part as desired. In some instances, the individual components, or fragments thereof, from different species (e.g., mouse, rat, human, etc.) may be combined, in whole or in part as desired to generate a chimeric or xeno-regulatory sequence. Furthermore, individual regulatory element may be further compacted to smaller or minimal functional elements for various reasons, e.g., to decrease the overall size of the resulting construct. Various methods for identifying the minimal functional elements of a regulatory element may be employed including but not limited to “promoter bashing”, “enhancer bashing”, in silico comparison with homologous/orthologous sequences to identify conserved domains, and the like.

### Encoded Polypeptides

The regulatory regions of the herein described expression cassettes may be operably linked to a sequence encoding one or more polypeptides such that activity-dependent activation of the regulatory region may drive expression of the encoded polypeptide. An encoded polypeptide operably linked to a regulatory region may be a protein derived from the same species as the regulatory region or the encoded polypeptide may be heterologous to the species from which regulatory region was derived, i.e., the encoded polypeptide may be derived from a species different from that of the regulatory region. In some instances, the encoded polypeptide may be wholly or partly synthetic, i.e., not derived from any naturally occurring peptide sequence. In some instances, the encoded polypeptide of a construct described herein may be a modified or mutated polypeptide, i.e., a polypeptide that has been modified or mutated as compared to its naturally occurring or wild-type form. In some instances, the encoded polypeptide may encode a wild-type protein though the nucleic acid encoding the wild-type protein may be modified from its wild-type form, e.g., the encoding sequence may be optimized for expression in a particular host, including e.g., where the encoding sequence is optimized for the codon usage of a particular host. As such, in some instances the encoding sequence may be “humanized” or “murinized”. Further modifications for mammalian and/or human and/or rodent expression or other purposes may be appended to the encoded proteins herein described including but not limited to e.g., an endoplasmic reticulum (ER) export signal, a nuclear localization signal (NLS), a cellular trafficking signal, etc.

Various encoded polypeptides may be expressed from the expression constructs of the instant disclosure including but not limited to e.g., light-responsive polypeptides, molecular tags, calcium or voltage sensors, ion channels, toxic proteins, receptors, nucleases, transcription factors, etc. Selection of a particular encoded polypeptide may depend on the end-use of the activity-dependent expression vector and/or the method within which it is employed. Subject encoded polypeptides may be described herein, in some instances, according to their expressed protein form; however, an ordinary skilled artisan will readily understand how the encoding nucleic acid sequence can be readily obtained or derived from such description.

In some instances, an encoded polypeptide of the instant disclosure may be a light-responsive polypeptide. As used herein the term “light-responsive polypeptide” refers to those polypeptides that undergo a conformational change, thus propagating a signal, in response to light exposure and may include but are not limited to e.g., those proteins useful in optogenetics (for review see e.g., Lerner & Deisseroth (2016) Cell. 164:1136-1150; Deisseroth (2015) Nat Neurosci. 18(9):1213-25; Buzsáki et al. (2015) Neuron. 86(1):92-105; Karunarathne et al. (2015) J Cell Sci. 128(1):15-25; McDevitt et al. (2014) Neuropsychiatr Dis Treat. 10:1369-79; Sidor et al. (2014) Front Behav Neurosci. 8:41; Xie et al. (2013) Acta Pharmacol Sin. 34(11):1381-5; Williams et al. (2013) Proc Natl Acad Sci USA. 110(41):16287; Touriño et al. (2013) Curr Opin Neurobiol. 23(3):430-5; Aston-Jones et al. (2013) Brain Res. 1511:1-5; Han et al. (2012) ACS Chem Neurosci. 3(8):577-84; Mei et al. (2012) Biol Psychiatry. 71(12):1033-8; Han et al. (2012) Prog Brain Res. 196:215-33; Zeng et al. (2012) Prog Brain Res. 196:193-213; Del Bene et al. (2012) Dev Neurobiol. 72(3):404-14; the disclosures of which are incorporated herein by reference in their entirety). Useful light-responsive polypeptides include but are not limited to e.g., opsins (e.g., depolarizing opsins, hyperpolarizing opsins, etc.) and those polypeptides described in PCT Publication Nos. WO2015/023782, WO2012/061744, WO2012/061684 and WO2015/148974; the disclosures of which, and their corresponding U.S. counterpart applications, are incorporated herein by reference in their entirety.

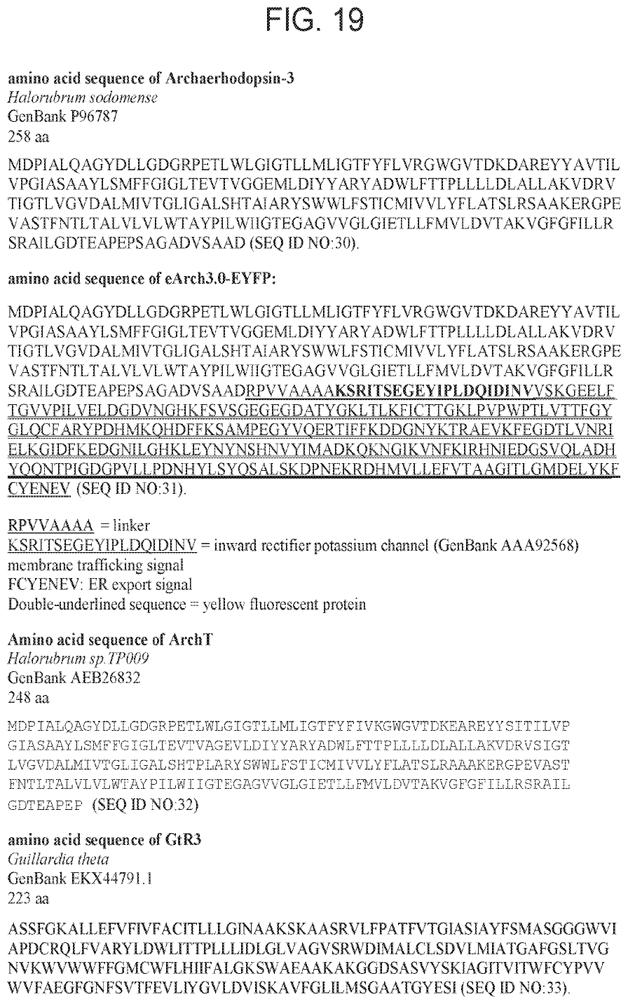

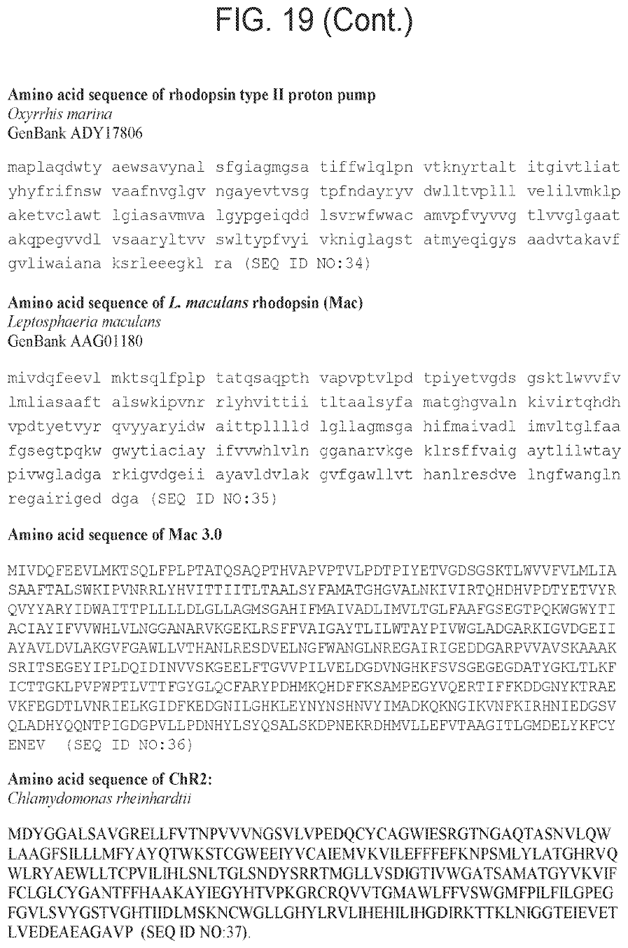

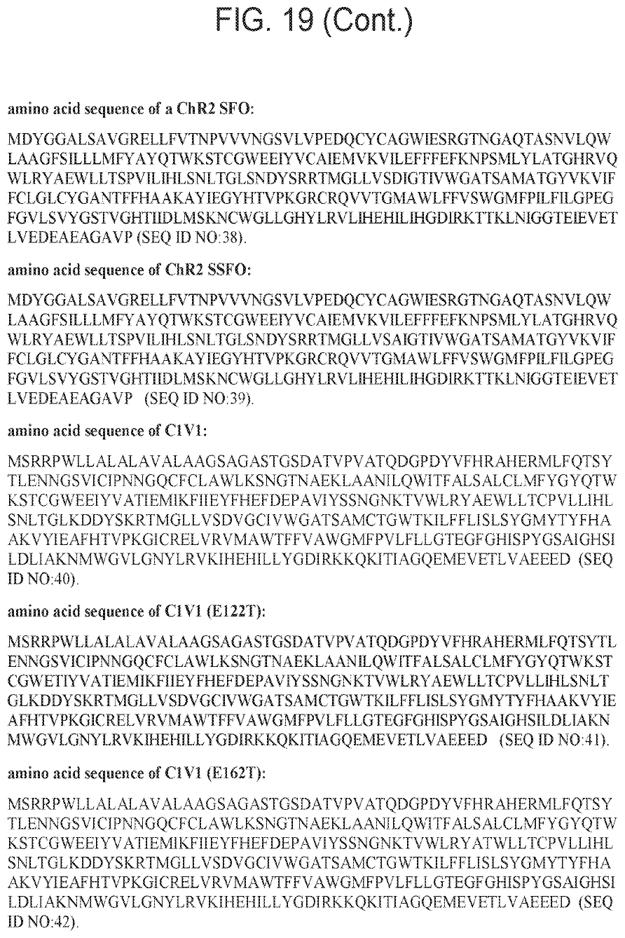

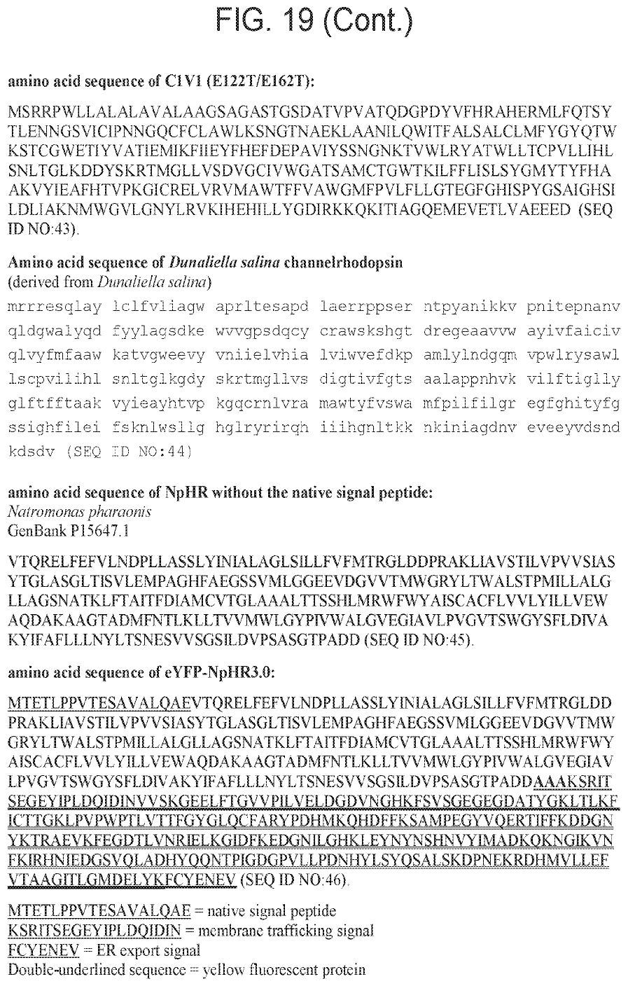

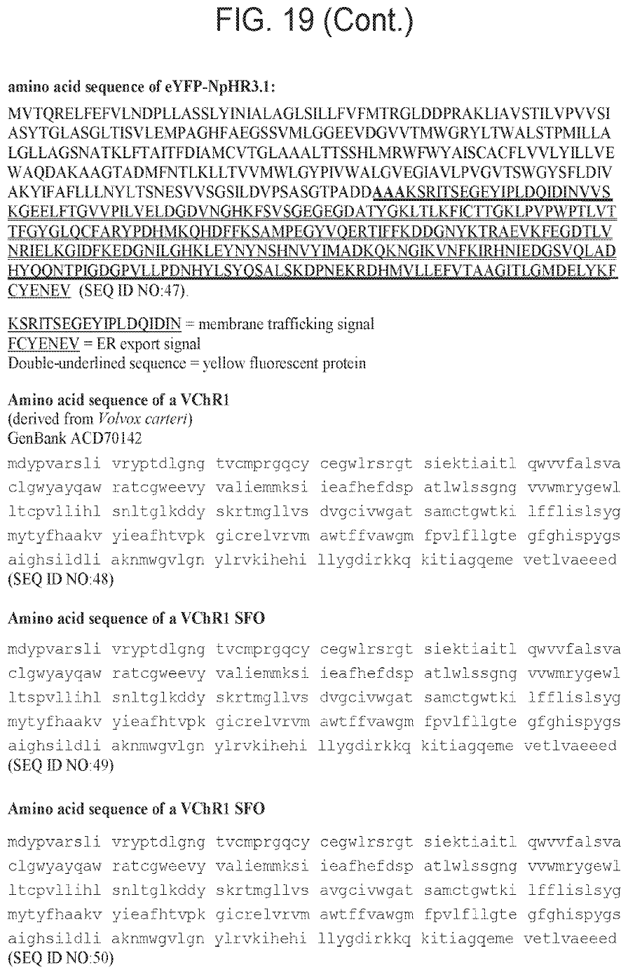

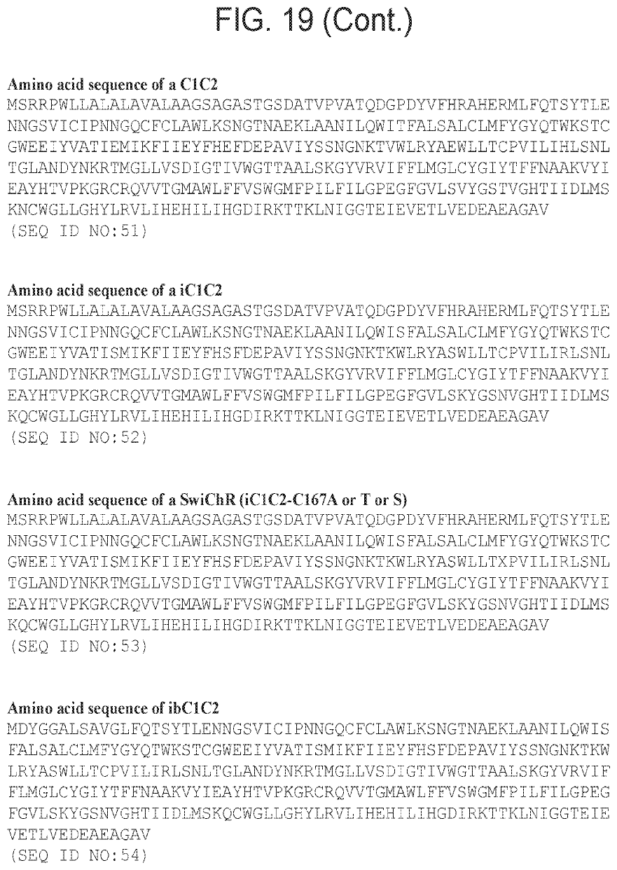

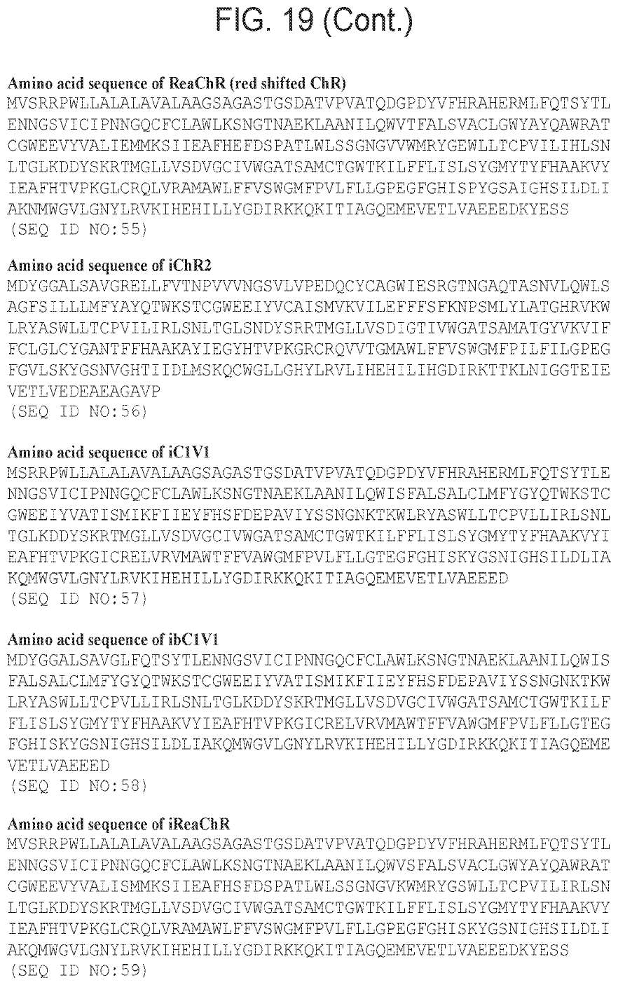

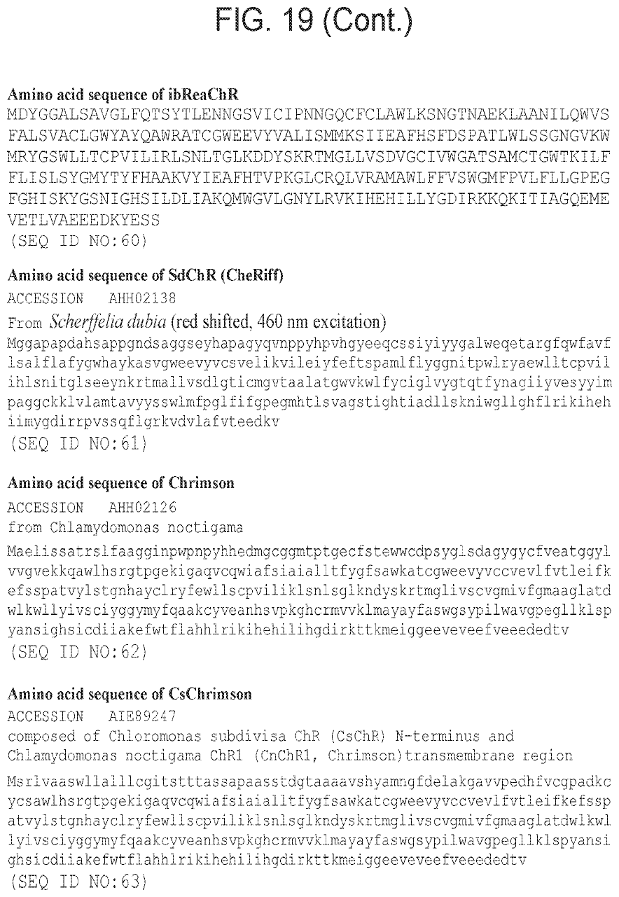

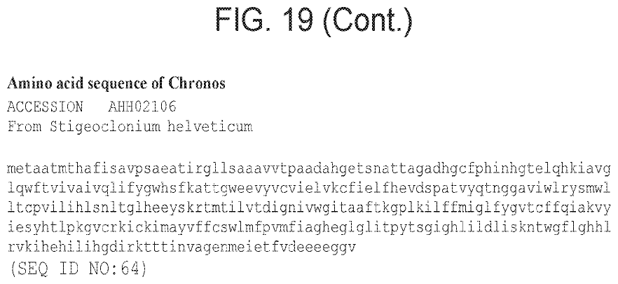

Useful light-responsive polypeptides include but are not limited to e.g., iC++ and SwiChR++ Next-generation engineered chloride-conducting channelrhodopsins, “bReaChES” Red-shifted optical excitation chimeric channelrhodopsins, SwiChR and iC1C2 action potential inhibition with chloride-conducting channelrhodopsins, Red-Shifted chimeric opsin variants (e.g., C1V1 variants), Stabilized Step Function Opsins (e.g., stabilized step function Ch R2 variants), Second-generation Ultrafast Optogenetic proteins (e.g., hChR2(T159C), hChR2(E123T/T159C), hChR2 (E123A), etc.), Third-generation Optogenetic Inhibition proteins (e.g., engineered halorhodopsin constructs (e.g., eNpHR 3.0), enhanced optical controllable proton pumps (e.g., those from H. sodomense (e.g., Arch), those from Halorubrum sp. TP009 (e.g., ArchT), those from L. maculans (e.g., Mac), etc.), Ultrafast Optogenetic Control proteins (e.g., ChETA), proteins for optical control of intracellular signaling (e.g., chimeric fusions of bovine Rhodopsin and adrenergic G-Protein Coupled Receptors allowing optical control of GPCR signaling cascades, also known as “Opto-XRs”), Bi-stable excitation ChR2 point-mutants providing a stable step in membrane potential (e.g., ChR2(C128A), ChR2(C128S), etc.), wild-type Channelrhodopsin-2 (ChR2) proteins, ChR2 mutants (hChR2(H134R), mammalian optimized Halorhodopsin (NpHR; also known as “eNpHR 2.0”), mammalian optimized Volvox Channelrhodopsin-1 (VChR1), and the like. In some instances, useful light-responsive polypeptides include but are not limited to e.g., those proteins and light-responsive constructs for which the amino acid sequences are provided in FIG. 19.

In some instances, useful light-responsive polypeptides may include fusion proteins between a light-responsive polypeptide and a fluorescent protein (including but not limited to e.g., those fluorescent proteins described herein). Any useful fluorescent protein fusion may be employed including e.g., a channelrhodopsin-fluorescent-protein fusion. In some instances, a useful light-responsive polypeptide fluorescent protein fusion may include but is not limited to a channelrhodopsin-fluorescent-protein fusion including e.g., Channelrhodopsin-2 (ChR2) fluorescent protein fusions including but not limited to e.g., ChR2-EGFP, ChR2-EYFP, ChR2-RFP, etc., including ChR2 fusions with any fluorescent protein including e.g., those fluorescent proteins described herein.

In some instances, an encoded polypeptide of the instant disclosure may be a molecular tag. As used herein the term “molecular tag” refers to a directly or indirectly detectable polypeptide expressed from a coding sequence. Such directly detectable polypeptides include but are not limited to e.g., fluorescent proteins, chromogenic proteins, etc. Indirectly detectable polypeptides include but are not limited to e.g., enzymes that catalyze a reaction with a substrate to produce a detectable product, affinity tags that allow detection through the binding of a binding partner (e.g., chitin binding protein (CBP), maltose binding protein (MBP), glutathione-S-transferase (GST), etc.) that is subsequently detected, epitope tags that allow detection through the binding of a an antibody directed to the epitope (e.g., anti-FLAG, anti-V5, anti-Myc, anti-HA, etc.) that is either directly detectable (e.g., through a fluorescent tag attached to the antibody) or indirectly detectable (e.g., through the binding of a secondary antibody, e.g., that is fluorescently labeled (i.e., a fluorescent secondary antibody).

Suitable chromogenic proteins include but are not limited to e.g., those available from DNA2.0 (Newark, Calif.), e.g., Blitzen Blue, Dreidel Teal, Virginia Violet, Vixen Purple, Prancer Purple, Tinsel Purple, Maccabee Purple, Donner Magenta, Cupid Pink, Seraphina Pink, Scrooge Orange, Leor Orange, those described in U.S. Pat. Nos. 8,975,042 and 9,290,552; the disclosures of which are incorporated herein by reference in their entirety, and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes for indirect detection include, but are not limited to, peroxidases (e.g., horse radish peroxidase (HRP)), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some instances, an encoded polypeptide of the instant disclosure may be a calcium sensor or voltage sensor or ion channel. Ion channels are membrane protein complexes and their function is to facilitate the diffusion of ions across biological membranes. In neurons, intracellular calcium signals have crucial roles in activating neurotransmitter release and in triggering alterations in neuronal function. Voltage-gated ion channels generate electrical signals in species from bacteria to man and their voltage-sensing modules are responsible for initiation of action potentials and graded membrane potential changes in response to synaptic input and other physiological stimuli.

Ion channels useful as an encoded polypeptide driven by an activity dependent regulatory region as described herein may include but are not limited to e.g., voltage-gated ion channels, ligand-gated ion channels, etc. Useful voltage-gated ion channels include but are not limited to e.g., calcium-activated potassium channels, CatSper and Two-Pore channels, cyclic nucleotide-regulated channels, inwardly rectifying potassium channels, ryanodine receptor channels, Transient Receptor Potential channels, Two-P potassium channels, Voltage-gated calcium channels, Voltage-gated potassium channels, Voltage-gated proton channels, Voltage-gated sodium channels, etc. Useful ligand-gated ion channels include but are not limited to e.g., 5-HT₃ receptors, Acid-sensing (proton-gated) ion channels (ASICs), Epithelial sodium channels (ENaC), GABA_{A} receptors, Glycine receptors, Ionotropic glutamate receptors, IP₃ receptors, Nicotinic acetylcholine receptors, P2X receptors, zinc activated ion channels, etc. Other ion channels include but are not limited to e.g., Aquaporins, Calcium activated chloride channels, cystic fibrosis transmembrane conductance regulator channels, CIC family channels, Connexins, Pannexins, Maxi chloride channels, non-selective sodium leak channels, volume regulated chloride channels, etc.

Calcium sensor proteins useful as an encoded polypeptide driven by an activity dependent regulatory region as described herein may include but are not limited to e.g., calmodulin, calnexin, calreticulin, gelsolin, Hippocalcin, Neurocalcin, Recoverin, neuronal calcium sensor (NCS) protein family members, Ca²⁺-binding proteins (CaBPs), and the like.

In some instances, an encoded polypeptide of the instant disclosure may be a toxic protein. The term “toxic proteins” as used herein generally refers to any protein that when expressed in a cell reduces cell viability or causes cell lethality. Thus, the term includes those proteins that are used to directly ablate cells (such as e.g., diphtheria toxic proteins) as well as those that may not directly induce toxicity but generally reduce viability (such as e.g., ribonucleases, deoxyribonucleases, proteases, etc.). Toxic proteins may be expressed within a host cell to serve various purposes including e.g., to impair or ablate or deplete the cell upon activity-dependent activation of the regulatory sequence of the expression construct. Any suitable and appropriate toxic protein may be utilized in an expression construct of the instant disclosure including but not limited to e.g., the A subunit of diphtheria toxin (DT-A), a ricin A subunit III, a herpes virus thymidme kinase, a M2(H37A) toxic ion channel, an E. coli nitroreductase gene (Ntr), a caspase, an expression product of cell death gene, and the like.

In some instances, an encoded polypeptide of the instant disclosure may be a receptor e.g., an extracellular receptor (e.g., G protein-coupled receptors, tyrosine and histidine kinase receptors, integrins, Toll gate and Toll-like receptors (e.g., TLR1, TLR2, TLR3, TLR4, TLR5, TLR6, TLR7, TLR8, TLR9, TLR10 and TLR11), ligand-gated ion channels, cytokine receptors (e.g., IL-2 family receptors, IL-3 family receptors, IL-6 family receptors, IL-12 family receptors, prolactin family receptors, interferon family receptors, IL-10 family receptors, Ig-like IL-1 family receptors, IL-17 family receptors, etc.) or an intracellular receptor (e.g., nuclear receptors (e.g., Thyroid hormone receptors, Retinoic acid receptors, Peroxisome proliferator-activated receptors, Rev-Erb receptors, Retinoic acid-related orphans, Liver X receptor-like receptors, Vitamin D receptor-like receptors, Hepatocyte nuclear factor-4 receptors, Retinoid X receptors, Testicular receptors, Tailless-like receptors, COUP-TF-like receptors, Estrogen-related receptors, Nerve growth factor IB-like receptors, Fushi tarazu F1-like receptors, Germ cell nuclear factor receptors, DAX-like receptors, etc.), cytoplasmic receptors, IP₃ receptors, etc.).

GPCRs useful as encoded polypeptides of the subject expression constructs include but are not limited to e.g., 5-Hydroxytryptamine receptors, Acetylcholine receptors, Adenosine receptors, Adrenoceptors, Angiotensin receptors, Apelin receptors, Bile acid receptors, Bombesin receptors, Bradykinin receptors, Cannabinoid receptors, Chemerin receptor, Chemokine receptors, Cholecystokinin receptors, Class A Orphans GPCRs, Complement peptide receptors, Dopamine receptors, Endothelin receptors, Formylpeptide receptors, Free fatty acid receptors, Galanin receptors, Ghrelin receptors, Glycoprotein hormone receptors, Gonadotrophin-releasing hormone receptors, GPR18, GPR55 and GPR119, G protein-coupled estrogen receptor, Histamine receptors, Hydroxycarboxylic acid receptors, Kisspeptin receptor, Leukotriene receptors, Lysophospholipid (LPA) receptors, Lysophospholipid (S1P) receptors, Melanin-concentrating hormone receptors, Melanocortin receptors, Melatonin receptors, Motilin receptor, Neuromedin U receptors, Neuropeptide FF/neuropeptide AF receptors, Neuropeptide S receptor, Neuropeptide W/neuropeptide B receptors, Neuropeptide Y receptors, Neurotensin receptors, Opioid receptors, Orexin receptors, Oxoglutarate receptor, P2Y receptors, Platelet-activating factor receptors, Prokineticin receptors, Prolactin-releasing peptide receptors, Prostanoid receptors, Proteinase-activated receptors, QRFP receptor, Relaxin family peptide receptors, Somatostatin receptors, Succinate receptors, Tachykinin receptors, Thyrotropin-releasing hormone receptors, Trace amine receptors, Urotensin receptors, Vasopressin and oxytocin receptors, Calcitonin receptors, Corticotropin-releasing factor receptors, Glucagon receptor family receptors, Parathyroid hormone receptors, VIP and PACAP receptors, Calcium-sensing receptors, Class C Orphan GPRC receptors, GABA_{B} receptors, Metabotropic glutamate receptors, Taste 1 receptors, Frizzled GPCRs, Adhesion GPCRs, and the like.

Useful receptor tyrosine kinases (RTK) include but are not limited to e.g., those of the following RTK subfamilies: Type I RTKs (ErbB (epidermal growth factor) receptor family), Type II RTKs (Insulin receptor family), Type III RTKs (PDGFR, CSFR, Kit, FLT3 receptor family), Type IV RTKs (VEGF (vascular endothelial growth factor) receptor family), Type V RTKs (FGF (fibroblast growth factor) receptor family), Type VI RTKs (PTK7/CCK4), Type VII RTKs (Neurotrophin receptor/Trk family), Type VIII RTKs (ROR family), Type IX RTKs (MuSK), Type X RTKs (HGF (hepatocyte growth factor) receptor family), Type XI RTKs (TAM (TYRO3-, AXL- and MER-TK) receptor family), Type XII RTKs (TIE family of angiopoietin receptors), Type XIII RTKs (Ephrin receptor family), Type XIV RTKs (RET), Type XV RTKs (RYK), Type XVI RTKs (DDR (collagen receptor) family), Type XVII RTKs (ROS receptors), Type XVIII RTKs (LMR family), Type XIX RTKs (Leukocyte tyrosine kinase (LTK) receptor family), Type XX RTKs (STYK1), etc.

Useful integrins include but are not limited to e.g., integrin α1β1, integrin α2β1, integrin αIIbβ3, integrin α4β1, integrin α4β7, integrin α5β1, integrin α6β1, integrin α10β1, integrin α11β1, integrin αEβ7, integrin αLβ2 and integrin αVβ3.

Useful receptors also include tumor necrosis factor (TNF) receptor superfamily (TNRSF) receptors which include but are not limited to e.g., TNFR1 (tumor necrosis factor receptor 1/TNFRSF1A), TNFR2 (tumor necrosis factor receptor 2/TNFRSF1B), lymphotoxin β receptor/TNFRSF3, OX40/TNFRSF4, CD40/TNFRSF5, Fas/TNFRSF6, decoy receptor 3/TNFRSF6B, CD27/TNFRSF7, CD30/TNFRSF8, 4-1 BB/TNFRSF9, DR4 (death receptor 4/TNFRSF10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACI/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17), GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27, ectodysplasin 1, anhidrotic receptor, and the like.

Useful receptors also include neurotransmitter receptors which include but are not limited to e.g., Adrenergic receptors (e.g., α1A, α1b, α1c, α1d, α2a, α2b, α2c, α2d, β1, β2, β3, etc.), Dopaminergic receptors (e.g., D1, D2, D3, D4, D5, etc.), GABAergic receptors (e.g., GABAA, GABAB1a, GABAB1δ, GABAB2, GABAC, etc.), Glutaminergic receptors (e.g., NMDA, AMPA, kainate, mGluR1, mGluR2, mGluR3, mGluR4, mGluR5, mGluR6, mGluR7, etc.), Histaminergic receptors (e.g., H1, H2, H3, etc.), Cholinergic receptors (e.g., Muscarinic receptors (e.g., M1, M2, M3, M4, M5; Nicotinic receptors (e.g., muscle, neuronal receptors (e.g., α-bungarotoxin-insensitive), neuronal receptors (e.g., α-bungarotoxin-sensitive), etc.), Opioid receptors (e.g., μ, δ1, δ2, κ, etc.), Serotonergic receptors (e.g., 5-HT1A, 5-HT1B, 5-HT1D, 5-HT1E, 5-HT1F, 5-HT2A, 5-HT2B, 5-HT2C, 5-HT3, 5-HT4, 5-HT5, 5-HT6, 5-HT7, etc.), Glycinergic receptors (e.g., Glycine, etc.), and the like.

In some instances, an encoded polypeptide of the instant disclosure may be a nuclease, including but not limited to e.g., site-specific nucleases that are useful, among other applications, in directed genome modification. Suitable site-specific nucleases include, but are not limited to, an RNA-guided DNA binding protein having nuclease activity, e.g., a Cas9 polypeptide; a transcription activator-like effector nuclease (TALEN); Zinc-finger nucleases; and the like.

Useful Cas9 polypeptides include but are not limited to e.g., those described in, e.g., Fonfara et al. (2014) Nucl. Acids Res. 42:2577; and Sander and Joung (2014) Nat. Biotechnol. 32:347; the disclosures of which are incorporated herein by reference in their entirety. A Cas9 polypeptide can comprise an amino acid sequence having at least about 75%, at least about 80%, at least about 85%, at least about 90%, at least about 95%, at least about 98%, or 100%, amino acid sequence identity to following Streptococcus pyogenes Cas9 amino acid sequence:

(SEQ ID NO: 25)
MDKKYSIGLDIGTNSVGWAVITDDYKVPSKKLKGLGNTDRHGIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLADSTDKVD
LRLIYLALAHMIKFRGHFLIEGDLNPDNSDVDKLFIQLVQTYNQLFEENP
INASRVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAT
LLSDILRVNSEITKAPLSASMIKRYDEHHQDLTLLKALVRQQLPEKYKEI
FFDQSKNGYAGYIDGGASQEEFYKFIKPILEKMDGTEELLAKLNREDLLR
KQRTFDNGSIPYQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSDILKEYP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKVGFIKRQLVETRQITKHVAQILDSRMNTKYDENDKLI
REVRVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKDPIDFLEAKGYKEVRKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

In some instances, a useful Cas9 polypeptide includes a Cas9 variant that lacks nuclease activity, but retains DNA target-binding activity. Such a Cas9 variant is referred to herein as a “dead Cas9” or “dCas9.” See, e.g., Qi et al. (2013) Cell 152:1173. A dCas9 polypeptide can comprise a D10A and/or an H840A amino acid substitution of SEQ ID NO:25 above or corresponding amino acids in another Cas9 polypeptide.

In some instances, a useful Cas9 polypeptide is a chimeric dCas9, e.g., a fusion protein comprising dCas9 and a fusion partner, where suitable fusion partners include, e.g., a non-Cas9 enzyme that provides for an enzymatic activity, where the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, suitable encoded Cas9 polypeptide is a chimeric dCas9, e.g., a fusion protein comprising dCas9 and a fusion partner, where suitable fusion partners include, e.g., a non-Cas9 enzyme that provides for an enzymatic activity, where the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

Useful nucleases may also include those described in e.g., Mishra, N C. Molecular Biology of Nucleases. Boca Raton, Fla.: CRC Press, Inc., 1995; Lim, S M & Lloyd R S. Nucleases. Plainview, N.Y.: Cold Spring Harbor Laboratory Press, 1993; the disclosures of which are incorporated herein by reference in their entirety.

In some instances, useful encoded polypeptides include recombinases, enzymes that catalyze the exchange of short pieces of DNA between two long DNA strands. Useful recombinases include but are not limited to e.g., Cre recombinase, Flp recombinase, PhiC31 integrase, and the like, including e.g., those recombinases described in Lodish H, et al. Molecular Cell Biology. 4^{th} ed. New York: W. H. Freeman; 2000; Olorunniji et al. (2016) Biochem J. 473(6):673-84 and Gaj et al. (2014) Biotechnol Bioeng. 111(1):1-15; the disclosures of which are incorporated herein by reference in their entirety.

In some instances, a useful recombinase in an activity-dependent expression construct of the instant disclosure includes a Cre recombinase. Useful Cre recombinases include but are not limited to e.g., those containing and/or derived from a protein of the following amino acid sequence

(SEQ ID NO: 26)
MSNLLTVHQNLPALPVDATSDEVRKNLMDMFRDRQAFSEHTWKMLLSVCR
SWAAWCKLNNRKWFPAEPEDVRDYLLYLQARGLAVKTIQQHLGQLNMLHR
RSGLPRPSDSNAVSLVMRRIRKENVDAGERAKQALAFERTDFDQVRSLME
NSDRCQDIRNLAFLGIAYNTLLRIAEIARIRVKDISRTDGGRMLIHIGRT
KTLVSTAGVEKALSLGVTKLVERWISVSGVADDPNNYLFCRVRKNGVAAP
SATSQLSTRALEGIFEATHRLIYGAKDDSGQRYLAWSGHSARVGAARDMA
RAGVSIPEIMQAGGWTNVNIVMNYIRNLDSETGAMVRLLEDGD

In some instances, a useful a recombinase will be a conditional recombinase including but not limited to e.g., those recombinases operably linked to a modified ligand-binding domain of the estrogen receptor (ER) that sequesters the recombinase outside of the nucleus until bound by an estrogen receptor antagonist (e.g., tamoxifen, 4-hydroxytamoxifen (4-OHT), etc.) (see e.g., Feil et al. (1997) BBRS 237:752-757; the disclosure of which is incorporated herein by reference in its entirety). Useful tamoxifen-inducible recombinases include but are not limited to e.g., inducible-Cre recombinases including but are not limited to e.g., Cre-ER^{T}(G521R), Cre-ER^{T2}, ERT2-Cre-ER^{T2}, etc., and those described in e.g., Hans et al. (2009) PLoS One 4(2): e4640; Boniface et al. (2009) Genesis 47(7):484; Seibler et al. (2003) Nucleic Acids Res. 31(4):e12; the disclosures of which are incorporated herein by reference in their entirety. The ER^{T2} domain is composed of amino acids 282-595 of the human estrogen receptor and carries three mutations (G400V/M543A/L544A). The human estrogen receptor isoform 1 amino acid sequence of RefSeq NP_000116.2 is provided below:

(SEQ ID NO: 27)
MTMTLHTKASGMALLHQIQGNELEPLNRPQLKIPLERPLGEVYLDSSKPA
VYNYPEGAAYEFNAAAAANAQVYGQTGLPYGPGSEAAAFGSNGLGGFPPL
NSVSPSPLMLLHPPPQLSPFLOPHGQQVPYYLENEPSGYTVREAGPPAFY
RPNSDNRRQGGRERLASTNDKGSMAMESAKETRYCAVCNDYASGYHYGVW
SCEGCKAFFKRSIQGHNDYMCPATNQCTIDKNRRKSCQACRLRKCYEVGM
MKGGIRKDRRGGRMLKHKRQRDDGEGRGEVGSAGDMRAANLWPSPLMIKR
SKKNSLALSLTADQMVSALLDAEPPILYSEYDPTRPFSEASMMGLLTNLA
DRELVHMINWAKRVPGFVDLTLHDQVHLLECAWLEILMIGLVWRSMEHPG
KLLFAPNLLLDRNQGKCVEGMVEIFDMLLATSSRFRMMNLQGEEFVCLKS
IILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRLH
APTSRGGASVEETDOSHLATAGSTSSHSLQKYYITGEAEGFPATV

In some instances, an encoded polypeptide of the instant disclosure may be a transcription factor. Useful transcription factors include but are not limited to e.g., AF-4 transcription factors, Androgen receptor transcription factors, AP-2 transcription factors, ARID transcription factors, bHLH transcription factors, C/EBP transcription factors, CBF transcription factors, CG-1 transcription factors, COE transcription factors, COUP transcription factors, CP2 transcription factors, CSD transcription factors, CSL transcription factors, CTF/NFI transcription factors, CUT transcription factors, DM transcription factors, E2F transcription factors, EAF2 transcription factors, Ecdystd receptor transcription factors, ETS transcription factors, Fork head transcription factors, GCM transcription factors, GCR transcription factors, GTF2I transcription factors, HMG transcription factors, HMGI/HMGY transcription factors, Homeobox transcription factors, HSF transcription factors, HTH transcription factors, IRF transcription factors, MBD transcription factors, MH1 transcription factors, MYB transcription factors, NDT80/PhoG transcription factors, NF-YA transcription factors, NF-YB/C transcription factors, Nrf1 transcription factors, Nuclear orphan receptor transcription factors, Oestrogen receptor transcription factors, P53 transcription factors, PAX transcription factors, PC4 transcription factors, POU transcription factors, PPAR receptor transcription factors, PREB transcription factors, Progesterone receptor transcription factors, Prox1 transcription factors, Retinoic acid receptor transcription factors, RFX transcription factors, RHD transcription factors, ROR receptor transcription factors, Runt transcription factors, SAND transcription factors, SPZ1 transcription factors, SRF transcription factors, STAT transcription factors, T-box transcription factors, TEA transcription factors, TF_bZIP transcription factors, TF_Otx transcription factors, THAP transcription factors, Thyroid hormone receptor transcription factors, TSC22 transcription factors, Tub transcription factors, ZBTB transcription factors, zf-BED transcription factors, zf-C2H2 transcription factors, zf-C2HC transcription factors, zf-GATA transcription factors, zf-LITAF-like transcription factors, zf-MIZ transcription factors, zf-NF-X1 transcription factors, and the like.

Many of the above described polypeptides may be combined either in a fusion construct or in a bicistronic construct for various useful applications. For example, an expressed protein having a cellular function may be tagged by fusion with a fluorescent protein (e.g., as described for various channelrhodopsins above) for identifying cells expressing the tagged protein. In some instances, a first polypeptide encoding sequence may be combined with a second polypeptide encoding sequence in a bicistronic construct (e.g., through the use of a 2A sequence (e.g., a p2A sequence from porcine teschovirus-1, a F2A sequence from the foot-and-mouth disease virus, a E2A sequence from equine rhinitis A virus sequence, a T2A sequence from Thosea asigna virus, etc.), including furin-2A sequences) to allow coordinated but separate production of both polypeptides from a single regulatory region within a cell. For example, in some instances a bicistronic cell-filling variant of an optogenetic construct may be employed where the construct includes sequence encoding a light-responsive polypeptide linked by a 2A (e.g., a p2A) to sequence encoding a fluorescent protein. Fusion constructs and bicistronic constructs are not limited to those specifically described and may be derived through combination of any (e.g., 2 or more, 3 or more, four or more, etc.) of the above described encoded polypeptides where appropriate.

In some instances, an encoded polypeptide of the instant disclosure may include an appended or attached PEST sequence (i.e., a peptide sequence that is rich in proline (P), glutamic acid (E), serine (S), and threonine (T)). Such PEST sequences are useful in decreasing the intracellular half-life of an expressed polypeptide. Useful PEST sequences include but are not limited to e.g., peptides encoded by the following sequence and variations thereof:

(SEQ ID NO: 28)
AGCCATGGCTTCCCGCCGGAGGTGGAGGAGCAGGATGATGGCACGCTGCC
CATGTCTTGTGCCCAGGAGAGCGGGATGGACCGTCACCCTGCAGCCTGTG
CTTCTGCTAGGATCAATGTG.

### Vectors

The instant disclosure provides vectors for the activity-dependent expression of encoded polypeptide sequences. Such vectors include but are not limited to e.g., plasmids (including e.g., episomal vectors, minicircle vectors, etc.), phage, transposons, cosmids, virus, etc., containing the expression constructs described herein.

A vector of the instant disclosure may include or exclude one or more vector specific elements. By “vector specific elements” is meant elements that are used in making, constructing, propagating, maintaining and/or assaying the vector before, during or after its construction and/or before its use, e.g., in a method of inducing activity-dependent expression of a desired encoded polypeptide. Such vector specific elements include but are not limited to, e.g., vector elements necessary for the propagation, cloning and selection of the vector during its use and may include but are not limited to, e.g., a vector backbone, an origin of replication, a multiple cloning site, a prokaryotic promoter, a phage promoter, sequence encoding one or more structural proteins, sequence encoding one or more envelope proteins, post-transcriptional regulatory machinery, a selectable marker (e.g., an antibiotic resistance gene, an encoded enzymatic protein, an encoded fluorescent or chromogenic protein, etc.), and the like. Any convenient vector specific elements may find use, as appropriate, in the vectors as described herein.

In some instances, useful vectors may include a plasmid containing an activity-dependent regulatory region as described herein for activity-dependent expression of a desired polypeptide and/or construction (e.g., cloning, virus production, etc.) of a secondary vector for activity-dependent expression of a desired polypeptide. Such plasmids may or may not contain sequence encoding the polypeptide of interest. For example, in some instances, a useful plasmid may contain a regulatory region adjacent to a cloning site (e.g., a multiple cloning site, a site-specific recombination site (e.g., an att site, etc.)) configured for the insertion of a desired polypeptide coding sequence. In some instances, a useful plasmid may already contain a regulatory region operably linked to a desired polypeptide coding sequence. In some instances, plasmid vector may be configured to be used directly to induce activity-dependent expression of a desired polypeptide as describe herein (e.g., through the direct transfection of the plasmid vector into a target cell of interest).

In some instances, plasmid vectors may be configured for the production of one or more recombinant viral vectors of the instant disclosure and may thus include sequence encoding viral components as described herein. In some instances, one or more components of needed for production of a viral vector may be provided in trans, i.e., provided by a separate plasmid. As such, in some instances, the necessary components for the production of recombinant virus may be split across two or more plasmids including but not limited to e.g., two plasmids, three plasmids, four plasmids, five plasmids, etc.

In some instances, useful vectors for regulatory region controlled activity-dependent expression of a desired polypeptide may be viral vectors, including recombinant viral vectors. Viral vectors will generally include a recombinant viral genome containing a regulatory region operably linked to a sequence encoding one or more polypeptides of interest.

Useful viral vectors include but are not limited to e.g., lentiviral vectors, HSV vectors, adenoviral vectors, and andeno-associated viral (AAV) vectors, and the like. Useful lentiviral vectors include those derived from HIV-1, HIV-2, SIV, FIV and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some instances, the vector is a recombinant AAV vector. AAV vectors are DNA viruses of relatively small size that can integrate, in a stable and site-specific manner, into the genome of the cells that they infect. They are able to infect a wide spectrum of cells without inducing significant effects on cellular growth, morphology or differentiation. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” in Parvoviruses (J R Kerr, S F Cotmore. M E Bloom, R M Linden, C R Parrish, Eds.) p5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and International Patent Application Publication No.: WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: WO 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some instances, useful AAV vectors for the expression constructs as described herein include those encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the instant disclosure includes a recombinant virus particle (recombinant because it contains a recombinant polynucleotide) comprising any of the vectors described herein. Methods of producing such particles are known in the art and are described in U.S. Pat. No. 6,596,535.

Depending on the type of vector utilized (e.g., whether the vector is a plasmid or viral vector) and the desired use of the vector, vectors as described herein may be formulated for use in a suitable container and/or medium in a variety of configurations. For example, in some instances, e.g., where the subject vector is a plasmid, the vector may be formulated in a dry (e.g., lyophilized) form or in a suitable solution such as e.g., water or buffer or culture medium. In some instances, vectors, including e.g., viral vectors may be provided in a ready-to-use format including e.g., where the vector is an AAV recombinant vector formulated in a ready-to-use format, e.g., configured for direct application or injection.

### Methods

The present disclosure provides methods for the activity-dependent expression of encoded polypeptides. Methods of the instant disclosure may make use of one or more of the expression constructs described herein and will generally include contacting a target cell with one or more of the subject expression constructs including, e.g., where the expression construct is within an expression vector. Upon activity-dependent activation of the regulatory region of a target cell contacted with an expression construct the target will express a polypeptide encoded by an encoding sequence operably linked to the regulatory region.

The term “activity-dependent activation”, particularly as it relates to the activation of a regulatory region as described herein, refers to a change in activation of a target cell due to an external input or stimulus on the target cell sufficient to induce or activate the subject regulatory region. For example, activity-dependent activation of a c-Fos regulatory region may include any input or stimulus on a target cell sufficient to activate a c-Fos regulatory region.

In some instances, e.g., where the target cell is a neuron, a stimulus sufficient for c-Fos regulatory region activation may include but is not limited to e.g., neuronal activation, including synaptic activation, electrophysiological activation and the like. In some instances, neuronal activation may be electrically induced e.g., by inducing an action potential through electrical stimulation of a neuron. In some instances, neuronal activation may be induced behaviorally, e.g., where an organism containing the subject neuron is allowed to perform or subjected to a particular behavior that activates the neuron. Useful behavioral stimulations include but are not limited to e.g., auditory stimulation, visual stimulation, an olfactory stimulation, avoidance/pain (e.g., shock, heat, cold, etc.) stimulation, gustatory stimulation, etc.). In some instances, neuronal activation may be induced pharmacologically, e.g., by contacting a neuron with, or administering to an organism containing a subject neuron, a pharmacological agent (e.g., an addictive and/or abused drugs including e.g., alcohol, club drugs (e.g., GHB, LSD, MDMA, Ketamine, methamphetamine, Rohypnol, etc.), cocaine, hallucinogens (e.g., LSD, Ketamine, PCP, Salvia, etc.), inhalants (i.e., psychoactive volatile substances), marijuana, opioids (heroine, hydrocodone, fentanyl, oxycodone, propoxyphene, hydromorphone, meperidine, diphenoxylate, etc.), central nervous system depressants (e.g., pentobarbital sodium, diazepam, alprazolam, etc.), stimulants (e.g., dextroamphetamine, methylphenidate, amphetamines, etc.), synthetic cannabinoids, synthetic cathinones, nicotine, etc.) that stimulates the neuron.

In some instances, activation of a c-Fos regulatory region may include contacting a cell, including neuronal and non-neuronal cells, with a c-Fos inducing agent. Useful c-Fos inducing agents include but are not limited to e.g., serum, growth factors (e.g., PDGF), lysophosphatidic acid, G proteins, etc. c-Fos inducing agents may also include those proteins, peptides and/or small molecules that activate elements present in c-Fos regulatory regions including but not limited to e.g., calcium cyclic AMP response element (CRE) inducing agents, serum response element (SRE) inducing agents, c-sis-platelet-derived growth factor (PDGF)-inducible factor element (SIE) inducing agents, etc.

The methods described herein may be performed in vitro or in vivo. For example, in some instances a subject target cell, including neuronal and non-neuronal cell types, may be contacted in vitro with an expression vector as described herein and subsequently stimulated, e.g., pharmacologically, electrically, etc., to induce activity dependent activation of a c-Fos regulatory region. In some instances, a cell with an activated c-Fos regulatory region may be referred to herein as an “activated cell” and, in other instances, an activated cell may refer to a target cell that has been subjected to an activating stimulus.

In some instances, a subject target cell, including neuronal and non-neuronal cell types, may be contacted in vivo with an expression vector, as described herein, e.g., by administering the expression vector to an organism containing the cell. Any convenient method of administering the expression vector in vivo may be utilized including e.g., those methods commonly employed for transfection of plasmids (e.g., electroporation, lipofection, biolistics, etc.), those methods commonly employed for infection of recombinant virus (e.g., injection, aerosol delivery, etc.). In some instances, following delivery of the subject expression vector to a host organism, the host organism may be exposed to a stimulus sufficient to activate a c-Fos regulatory region of the expression vector, including but not limited to e.g., a pharmacological stimulus, an electrical stimulus, a physical (e.g., touch, pain, etc.) stimulus, a visual stimulus, an auditory stimulus, an olfactory stimulus, a gustatory stimulus, a behavioral stimulus, etc.

In some instances, whether the method is performed in vitro or in vivo, the subject cell may be maintained under conditions permissive for activity dependent activation. By “permissive for activity dependent activation” is meant that the cell is kept in a state, following exposure or infection with an expression construct as described herein, such that the cell is capable of responding to a stimulus sufficient to activate the regulatory region of the expression construct. For example, in instances where the method is performed in vitro, maintaining a cell under conditions permissive for activity dependent activation may include but is not limited to e.g., culturing the cell under established culture conditions for the particular cell type (e.g., providing sufficient culture medium, temperature, CO₂, etc., to maintain the viability of the cell). In instances where the method is performed in vivo, maintaining a cell under conditions permissive for activity dependent activation may include but is not limited to e.g., maintaining the organism harboring the cell under environmental conditions sufficient to maintain the viability of the host organism. Conditions permissive for activity dependent activation will also be configured such that the cell or organism harboring the cell is capable of responding to a regulatory region inducing stimulus provided to activate the cell.

Methods of the instant disclosure include methods for activity-dependent labeling of an activated cell using an activity-dependent expression construct as described herein. For example, in some instances, a cell may be contacted with an expression construct configured for activity-dependent labeling and subsequently activated to label the cell.

Useful constructs for activity-dependent labeling include but are not limited to e.g., a construct expressing a molecular tag under control of an activity-dependent regulatory region. For example, a cell may be contacted with an expression construct that includes a fluorescent protein under control of an activity-dependent regulatory region such that, upon exposure to a stimulus, the regulatory region is activated and the fluorescent protein is expressed thus labeling the cell. In some instances, accumulation of a molecular tag is controlled, e.g., by expressing a degradation signal e.g., a PEST sequence in operable linkage with the molecular tag.

Useful constructs for activity-dependent labeling include but are not limited to e.g., a construct expressing a recombinase under control of an activity-dependent regulatory region. For example, a cell may be contacted with an expression construct that includes a recombinase under control of an activity-dependent regulatory region such that, upon exposure to a stimulus, the regulatory region is activated and the recombinase recombines a genetic element within the cell thus labeling the cell. In some instances, the cell is configured to contain a molecular tag sequence that is not expressed prior to recombination and, following recombination, the molecular tag is expressed. In some instances, the cell is configured to contain a molecular tag sequence that is expressed prior to recombination and, following recombination, the molecular tag is not expressed. Toggling of expression of a molecular tag within a subject cell by an activity-dependent expressed recombinase may be achieved by a variety of ways including e.g., by flanking a genetic stop adjacent to the molecular tag encoding sequence with recombination sites (e.g., loxP sites) such that following recombination of the sites the molecular tag is expressed, flanking a molecular tag with recombination sites such that following recombination of the sites the molecular tag is no longer expressed. Labeling of target cells through a recombination event may, in some instances, allow for the prolonged labeling of the target cell including, e.g., continued expression of the label even after the c-Fos regulatory region is no longer active.

In some instances, the methods described herein may involve contacting a conditional reporter mouse with an activity-dependent expression vector. Useful conditional reporter mice (e.g., mice with “floxed” alleles allowing the toggling of expression of a reporter upon expression of a recombinase) include but are not limited to e.g., B6;129S6-Gt(ROSA)26Sor^{tm1 (CAG-tdTomato) Hze}/J (a.k.a. Ai14) mice, B6;129S4-Gt(ROSA)26Sor^{tm3 (CAG-tdTomato,-EGFP*)Zjh}/J mice, B6;129S4-Gt(ROSA)26Sor^{tm4(CAG-mOrange2,-EGFP,-mKate2)Zjh}/J mice, B6.Cg-Gt(ROSA)26Sor^{tm9(CAG-tdTomato)Hze}/J (a.k.a. Ai9) mice, B6.129P2-Gt(ROSA)26Sor^{tm1(CAG-Brainbow2.1)Cle}/J mice, and the like.

Activity-dependent expression of a recombinase may be performed for purposes other than cell labeling and such purposes may vary greatly. Various genes, both autologous and heterologous, may be activated and/or deactivated in response to cellular activity through the activity-dependent expression of a recombinase as described herein. For example, any convenient conditional (e.g., “floxed”) rodent line may be employed for activity dependent control of the conditional allele according to the methods as described herein. Useful mouse conditional mouse lines include but are not limited to e.g., those conditionally expressing CRISPR/Cas9 (e.g., B6;129-Gt(ROSA)26Sor^{tm1(CAG-cas9*,-EGFP) Fezh}/J, etc.), those conditionally expressing components allowing for conditional ablation (e.g., C57BL/6-Gt(ROSA)26Sor^{tm1(HBEGF) Awai}/J, etc.), those conditionally repressing nervous system genes (e.g., B6;SJL-NIgn2^{tm1.1Sud}/J, C57BL/6N-Tg(Npy-EGFP/RNAi:Gad1)1Mirn/J, 129-Dag1^{tm2Kcam}/J, B6(Cg)-Syde1^{tm1c(EUCOMM)Hmgu}/ScheiJ, etc.), and the like.

In some instances, an organism expressing a activity-dependent expression construct sufficient for the activity-dependent labeling of neurons may be used to identify stimuli sufficient to activate neurons including e.g., specific neurons related to desirable or undesirable biological functions or behaviors. For example, a neuron expressing a cell activity reporter may be exposed to various stimuli and neuronal activation may be screened for. In such a manner, many compounds may be screened for a role in activating neurons generally or activation of specific neurons through the use of a cell and/or an animal (e.g., a rat or mouse) expressing an activity dependent reporter as described herein. In addition to screening pharmacological compounds, other stimuli, including e.g., those described herein, can be screened for an activation-effect on neurons generally or on specific sets of or individual neurons.

Methods of the instant disclosure include methods for activity-dependent control of an activated cell using an activity-dependent expression construct as described herein. For example, in some instances, a cell may be contacted with an expression construct configured for activity-dependent control and subsequently activated to control the cell.

Useful constructs for activity-dependent control include but are not limited to e.g., a construct expressing a light-responsive polypeptide under control of an activity-dependent regulatory region. For example, a cell may be contacted with an expression construct that includes a channelrhodopsin under control of an activity-dependent regulatory region such that, upon exposure to a stimulus, the regulatory region is activated and the channelrhodopsin protein is expressed thus allowing the cell the be controlled by subsequent exposure to light. In some instances, accumulation of an expressed light-responsive polypeptide is controlled, e.g., by expressing a degradation signal e.g., a PEST sequence in operable linkage with the light-responsive polypeptide.

Useful light-responsive polypeptides for light-mediate control of an activated cell include but are not limited to those light-responsive polypeptides described herein. In some instances, following activation of a c-Fos regulatory region by exposure of a subject cell to a stimulus a light-responsive polypeptide is expressed in the activated cell allowing for hyperpolarization of the cell upon exposure to light. In some instances, following activation of a c-Fos regulatory region by exposure of a subject cell to a stimulus a light-responsive polypeptide is expressed in the activated cell allowing for depolarization of the cell upon exposure to light.

In some instances, the subject methods, where light-responsive polypeptides are expressed in an activity-dependent manner, allows for conditional control over all neurons activated in response to a particular stimulus. Accordingly, in some instances, all or the majority of neurons activated in response to a pharmacological stimulus may be reactivated or deactivated upon exposure to light according to the methods described herein. In some instances, all or the majority of neurons activated in response to a behavioral stimulus may be reactivated or deactivated upon exposure to light according to the methods described herein. Any convenient and appropriate method of exposing the activated cells to light may be employed including but not limited to e.g., fiber-optic lights, lasers, fluorescent light, incandescent light, etc., where the light may be of a broad band of wavelengths or a constrained band of wavelengths or essentially a single wavelength.

In some instances, methods for activity dependent labeling may be combined with methods for activity dependent control. For example, in some instances, a single activity-dependent regularly region may be employed to drive expression of both a molecular tag and a light-responsive polypeptide such that, upon activation, the active cell may be both labeled and controllable. In some instances, two separate activity-dependent regularly regions may be employed, including where two separate expression cassettes and/or two separate expression vectors are employed, to drive expression of a molecular tag and a light-responsive polypeptide such that, upon activation, the active cell may be both labeled and controllable. Various combinations of the subject expression constructs and vectors may be employed in the methods as described for labeling and/or controlling and/or modifying target cells in an activity-dependent manner.

Such combinations of expression constructs and/or expression vectors may be described herein as systems, including e.g., expression systems, where a system may include two or more different expression constructs or vectors. The two constructs or vectors of a system may be configured to work in concert to serve a particular purpose, e.g., to allow for efficient control of an activated cell, to allow for efficient labeling of an activated cell, to allow for simultaneous control and labeling of an activated cell, to allow for efficient modulation of an activated cell, etc.

Target cells of the subject methods will vary depending on the desired purpose for activity-dependent expression as described herein. In some cases, the cell is a mammalian cell. In some cases, the cell is a human cell. In some cases, the cell is a non-human primate cell. In some cases, the cell is rodent cell. In some cases, the cell is mouse cell. In some cases, the cell is a rat cell.

Suitable cells include retinal cells (e.g., Müller cells, ganglion cells, amacrine cells, horizontal cells, bipolar cells, and photoreceptor cells including rods and cones, Müller glial cells, and retinal pigmented epithelium); neural cells (e.g., cells of the thalamus, sensory cortex, zona incerta (ZI), ventral tegmental area (VTA), prefontal cortex (PFC), nucleus accumbens (NAc), amygdala (BLA), substantia nigra, ventral pallidum, globus pallidus, dorsal striatum, ventral striatum, subthalamic nucleus, hippocampus, dentate gyrus, cingulate gyrus, entorhinal cortex, olfactory cortex, primary motor cortex, or cerebellum); liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs)

In some cases, the stem cell is a hematopoietic stem cell (HSC), and the transcription factor induces differentiation of the HSC to differentiate into a red blood cell, a platelet, a lymphocyte, a monocyte, a neutrophil, a basophil, or an eosinophil. In some cases, the stem cell is a mesenchymal stem cell (MSC), and the transcription factor induces differentiation of the MSC into a connective tissue cell such as a cell of the bone, cartilage, smooth muscle, tendon, ligament, stroma, marrow, dermis, or fat.

In some cases, the cell is a cancer cell. In some cases, the cancer cell is a carcinoma cancer cell, a sarcoma cancer cell, a lymphoma cancer cell, a germ cell tumor cancer cell, a blastoma cancer cell, or the like.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-49 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An expression vector comprising, an activity-dependent expression cassette comprising:

(a) a regulatory sequence comprising a c-Fos 5′-non-coding region and a c-Fos first intron sequence; and

(b) a polypeptide coding sequence operably linked to the regulatory sequence, wherein the polypeptide encoded by the polypeptide coding sequence is expressed from the expression cassette upon activity-dependent activation of the regulatory sequence.

2. The expression vector of 1, wherein the vector is a viral vector.

The expression vector of 2, wherein the viral vector is a recombinant adeno-associated virus (AAV) vector.

4. The expression vector of any of 1-3, wherein the regulatory sequence is a mammalian c-fos regulatory sequence comprising a mammalian c-Fos 5′-non-coding region and a mammalian c-Fos first intron sequence.

5. The expression vector of 4, wherein the mammalian c-fos regulatory sequence is a rodent c-fos regulatory sequence comprising a rodent c-Fos 5′-non-coding region and a rodent c-Fos first intron sequence.

6. The expression vector of 5, wherein the rodent c-fos regulatory sequence is a mouse c-fos regulatory sequence comprising a mouse c-Fos 5′-non-coding region and a mouse c-Fos first intron sequence.

7. The expression vector of any of 1-6, wherein the expression cassette further comprises a sequence encoding a PEST peptide operably linked to the 3′ end of the polypeptide coding sequence.

8. The expression vector of any of 1-7, wherein the polypeptide coding sequence is heterologous to the c-fos regulatory sequence.

9. The expression vector of any of 1-8, wherein the polypeptide coding sequence encodes a light-responsive polypeptide.

10. The expression vector of 9, wherein the light-responsive polypeptide is a depolarizing opsin or a hyperpolarizing opsin.

11. The expression vector of any of 1-8, wherein the polypeptide coding sequence encodes a molecular tag.

12. The expression vector of any of 1-8, wherein the polypeptide coding sequence encodes a calcium sensor or voltage sensor or ion channel.

13. The expression vector of any of 1-8, wherein the polypeptide coding sequence encodes a toxic protein.

14. The expression vector of any of 1-8, wherein the polypeptide coding sequence encodes a receptor.

15. The expression vector of any of 1-8, wherein the polypeptide coding sequence encodes a nuclease.

16. The expression vector of any of 1-8, wherein the polypeptide coding sequence encodes a transcription factor.

17. The expression vector of any of 1-16, wherein the polypeptide coding sequence encodes a fusion protein comprising two or more polypeptides selected from the group consisting of: a light-responsive polypeptide, a molecular tag, a calcium sensor or voltage sensor or ion channel, a toxic protein, a receptor, a nuclease and a transcription factor.

18. The expression vector of any of 1-17, wherein the c-Fos 5′-non-coding region is less than 800 nucleotides in length.

19. The expression vector of 18, wherein the c-Fos 5′-non-coding region has a sequence identity of 80% or greater with SEQ ID NO:1.

20. The expression vector of any of 1-19, wherein the c-Fos first intron sequence comprises the entire first intron of a c-Fos gene or a degenerate sequence thereof.

21. The expression vector of any of 1-20, wherein the c-Fos first intron has a sequence identity of 80% or greater with SEQ ID NO:2.

22. The expression vector of any of 1-21, wherein the expression cassette further comprises a sequence of 50 to 200 nucleotides length positioned between the c-Fos 5′-non-coding region and the c-Fos first intron sequence.

23. The expression vector of 22, wherein the sequence of 50 to 200 nucleotides length comprises a sequence encoding the first exon of a c-Fos gene or a portion thereof.

24. The expression vector of 23, wherein the sequence encoding the first exon of a c-Fos gene has a sequence identity of 80% or greater with SEQ ID NO:3.

25. A recombinant adeno-associated virus (AAV), comprising an expression vector according to any of 1-24.

26. A method for activity-dependent labeling of an active cell, the method comprising:

(a) contacting a cell with an expression vector comprising an expression cassette comprising:

(i) a regulatory sequence comprising a c-Fos 5′-non-coding region and a c-Fos first intron sequence; and

(ii) a coding sequence encoding a labeling polypeptide operably linked to the regulatory sequence; and

(b) maintaining the cell under conditions permissive for activity-dependent activation of the regulatory sequence, wherein upon activity-dependent activation of the regulatory sequence the labeling polypeptide is expressed labeling the active cell.

27. The method of 26, wherein the contacting is performed in vitro.

28. The method of 26, wherein the contacting is performed in vivo.

29. The method according to any of 26-28, wherein the cell is a neuron.

30. The method according to 29, wherein the neuron is a mammalian neuron.

31. The method according to any of 29-30, wherein the neuron is present in the central nervous system of a vertebrate.

32. The method according to any of 26-31, wherein during the maintaining the cell is contacted with a stimulus thereby activating the regulatory sequence.

33. The method according to 32, wherein the stimulus is an electrical stimulus.

34. The method according to 32, wherein the stimulus is a pharmacological stimulus.

35. The method according to any of 26-34, wherein the contacting is performed in vivo by administering the expression vector to the central nervous system of a vertebrate and the maintaining comprises subjecting the vertebrate to a behavioral task sufficient to activate the regulatory sequence.

36. The method according to any of 26-35, wherein the labeling polypeptide is a molecular tag.

37. The method according to any of 26-36, wherein the labeling polypeptide is a recombinase and the cell comprises a recombination sequence that, upon recombination, induces expression of a molecular tag.

38. A method for activity-dependent control of an activated cell, the method comprising:

(a) contacting a cell with an expression vector comprising an expression cassette comprising:

(i) a regulatory sequence comprising a c-Fos 5′-non-coding region and a c-Fos first intron sequence; and

(ii) a coding sequence encoding a light-responsive polypeptide operably linked to the regulatory sequence;

(b) maintaining the cell under conditions permissive for activity-dependent activation of the regulatory sequence, wherein upon activity-dependent activation of the regulatory sequence the light-responsive polypeptide is expressed in the activated cell; and

(c) exposing the activated cell to light sufficient to trigger the light-responsive polypeptide to induce a response in the cell thereby controlling the activated cell.

39. The method of 38, wherein the contacting is performed in vitro.

40. The method of 39, wherein the contacting is performed in vivo.

41. The method according to any of 38-40, wherein the cell is a neuron.

42. The method according to 41, wherein the neuron is a mammalian neuron.

43. The method according to any of 38-42, wherein the neuron is present in the central nervous system of a vertebrate.

44. The method according to any of 38-43, wherein during the maintaining the cell is contacted with a stimulus thereby activating the regulatory sequence.

45. The method according to 44, wherein the stimulus is an electrical stimulus.

46. The method according to 44, wherein the stimulus is a pharmacological stimulus.

47. The method according to any of 38-46, wherein the contacting is performed in vivo by administering the expression vector to the central nervous system of a vertebrate and the maintaining comprises subjecting the vertebrate to a behavioral task sufficient to activate the regulatory sequence.

48. The method according to any of 38-47, wherein the response is depolarization.

49. The method according to any of 38-47, wherein the response is hyperpolarization.

### EXAMPLES

### Materials and Methods:

### Animals

Male and female C57BL/6J mice were group-housed on a reverse 12 h light/dark cycle. Mice were 6 to 8 weeks old at the time of viral infusion. Food and water were given ad libitum. Ai14 mice and wild type C57BL/6 mice were purchased from JAX. Rosa26^{lxp-stop-loxp-eGFP-L10} (referred to as rTag herein)mice obtained from academic sources. Male mice were used in all behavioral assays. Both male and female mice were used for histology and anatomy assays. All experimental protocols were approved by the Stanford University Institutional Animal Care and Use Committee and were in accordance with the guidelines from the National Institutes of Health.

### Virus and Injection

Adeno-associated viral (AAV) vectors were serotyped with AAV5 or AAV8 coat proteins and packaged. Injections were made unilaterally into the PFC with final viral concentrations of AAV8-fos-ER^{T2}-Cre-ER^{T2}-PEST: 3×10¹², AAV8-CaMKIIα-EYFP-NRN: 1.5×10¹², AAV5-fosCh-YFP: 2×10¹², AAV5-CaMKIIα-YFP: 1.5×10¹¹, all as genome copies per mL.

### Constructs and Virus

The pAAV-fos-ChR2-EYFP (fosCh) plasmid was constructed by fusing the codon-optimized ChR2 (H134R) tagged with enhanced yellow fluorescent protein to a truncated c-fos gene sequence that included the 767 bp minimal promoter segment and the 500 bp intron 1 coding region containing key regulatory elements. A 70 bp PEST sequence was inserted at the C-terminal end to promote degradation and thereby prevent the membrane targeted ChR2-YFP from accumulating over time. The construct was cloned into an AAV backbone. The pAAV-fos-ER^{T2}-Cre-ER^{T2}-PEST plasmid was constructed by replacing the ChR2-EYFP in the fosCh plasmids with an ER^{T2}-Cre-ER^{T2} cassette. The pAAV-CaMKIIα-EYFP-NRN plasmid was constructed by replacing the 479 bp hGH polyA tail in pAAV-CaMKIIα-eYFP-WPRE-hGHpa with a DNA fragment containing the 992 bp 3′ UTR of Neuritin plus 215 bp bGH poly A flanked by AfeI and BstEI sites (NRN from the 3′ UTR of the rat neuritin mRNA, (NM_053346.1)).

### CAPTURE Labeling

Ai14 mice were injected with 1 μl mixture of AAV8-CaMKIIα-EYFP-NRN and AAV8-cFos-ER-Cre-ER-PEST in the left side of the mPFC. Two weeks after surgery, the mice were given 15 mg/kg cocaine (IP injection) or 20 random foot shocks (2 s, 0.5 mA, 2 shocks per minute on average) for two consecutive days. The control group remained in their home cage for the whole period. 10 mg/kg 4-hydroxytamoxifen was given to all mice 3 hours after the last behavior section to enable CreER-mediated recombination. The mice were returned to their home cage for additional 3-4 weeks to allow the full expression of fluorescence protein.

### Stereotaxic Surgery

6-7-week-old mice were anaesthetized with 1.5-3.0% isoflurane and placed in a stereotaxic apparatus (Kopf Instruments). Surgeries were performed under aseptic conditions. A scalpel was used to open an incision along the midline to expose the skull. After performing a craniotomy, viruses (specific titer and volume for each virus can be found in the virus preparation section) was injected into the mPFC using a 10 μl nanofill syringe (World Precision Instruments) at 0.1 μl min-1. The syringe was coupled to a 33 gauge beveled needle, and the bevel was placed to face the anterior side of the animal. The syringe was slowly retracted 20 min after the start of the infusion. A slow infusion rate followed by 10 min of waiting before retracting the syringe was crucial to restrict viral expression to the target area. Infusion coordinates were: anteroposterior, 1.9 mm; mediolateral, 0.35 mm; dorsoventral, 2.6 mm. Coordinates for the unilateral implantation of fiber optic cannulas (Doric Lenses 200 μm diameter) were: anteroposterior, 1.9 mm; mediolateral, 0.35 mm; dorsoventral, −2.4 mm. All coordinates relative to bregma.

### Delivery of 4-Hydroxytamoxifen

An aqueous formulation (instead of oil, which tends to give slower drug release) is designed to facilitate transient 4TM delivery. 10 mg of 4TM (Sigma H6278) was first dissolved in 250 μl DMSO. This stock is first diluted in 5 ml of saline containing 2% Tween 80 and then diluted 1:1 again with saline. The final injectable solution contained: 1 mg/ml 4TM, 1% Tween 80 and 2.5% DMSO in saline. The pharmacokinetics of 4TM in mouse brain (using the above vehicle) was determined using a standard LS-MS method at Biomaterials and Advanced Drug Delivery Laboratory at Stanford. Briefly, 30 C57BL/6J mice were injected (IP) with 10 mg/kg 4TM at indicated time points (n=5 each time point) and n=5 mice injected with vehicle alone were used as blank control. Brains were collected after perfusion using 1×PBS at different time points and snap-frozen in liquid nitrogen before homogenized for Liquid Chromatography Mass Spectrometry (LC-MS) analysis.

### CLARITY Processing

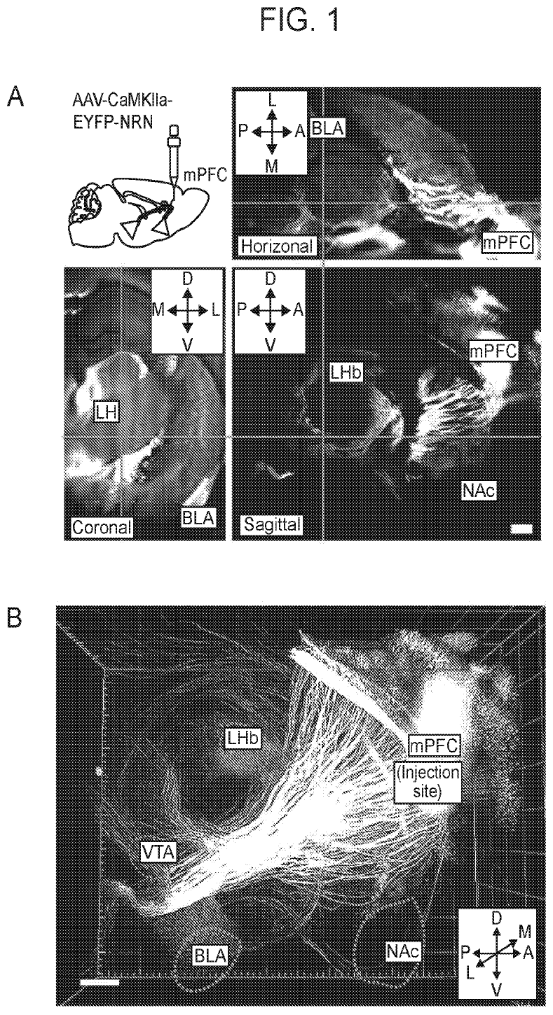

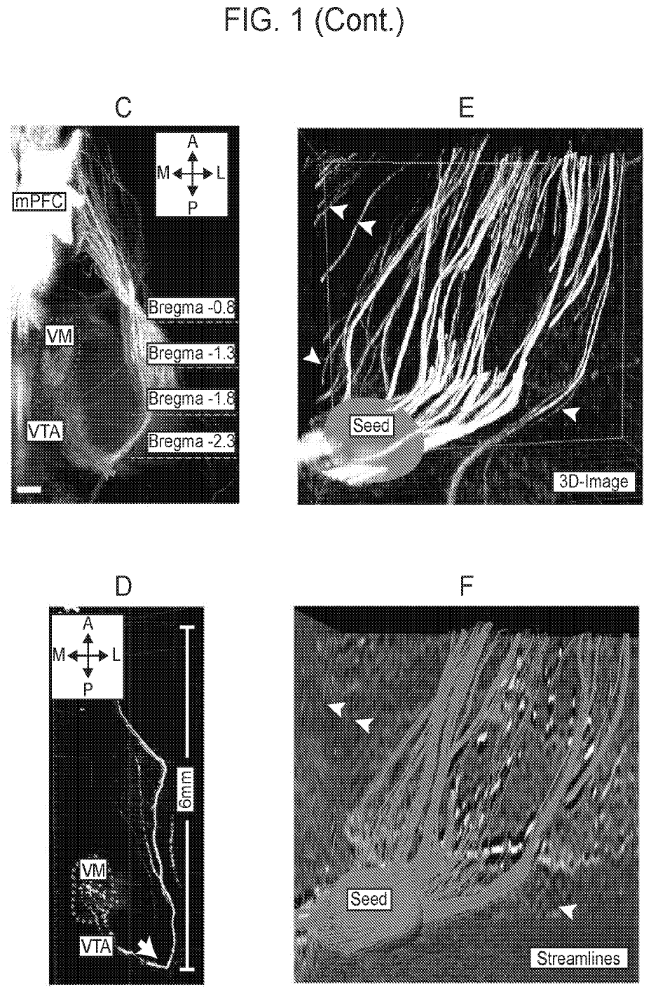

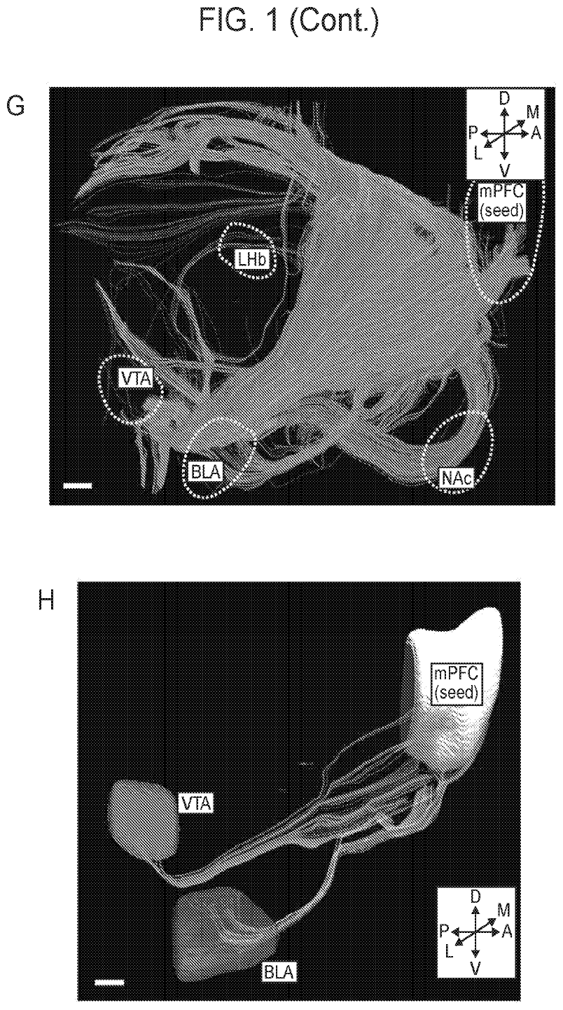

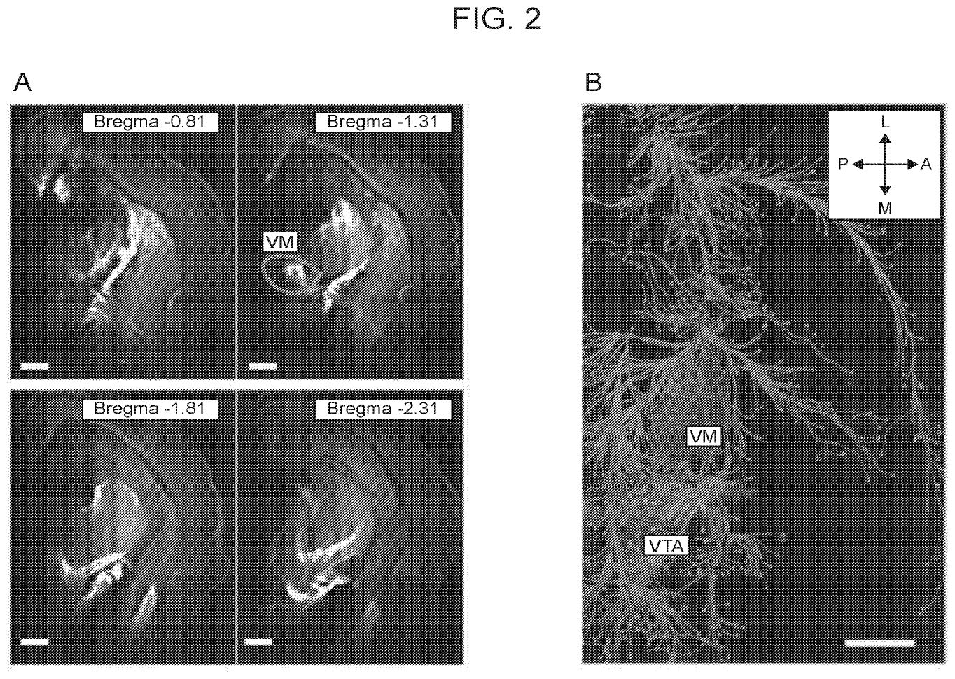

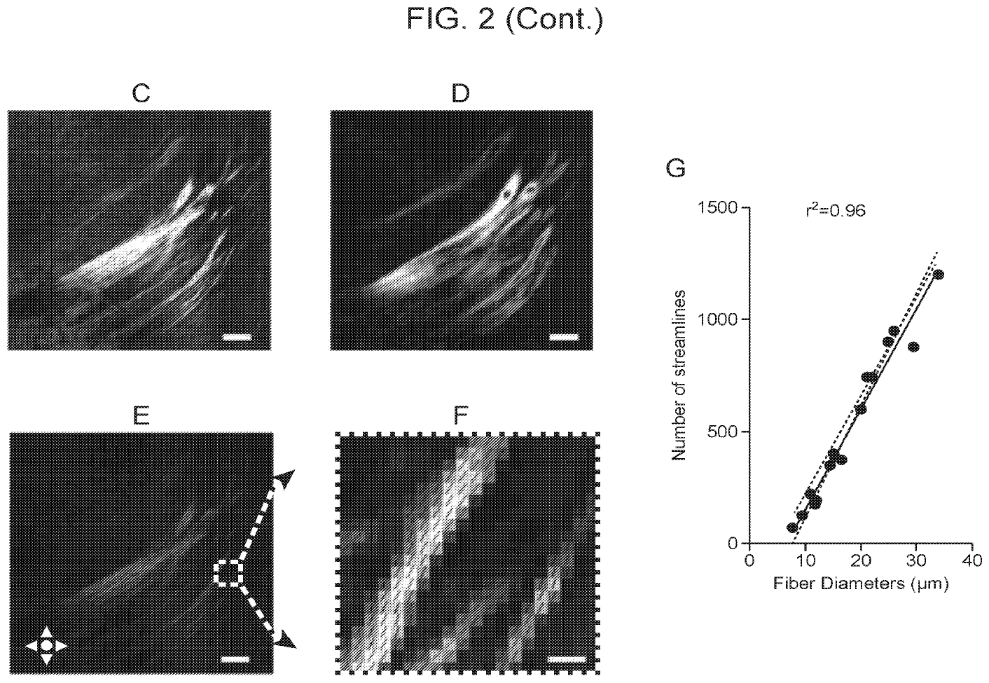

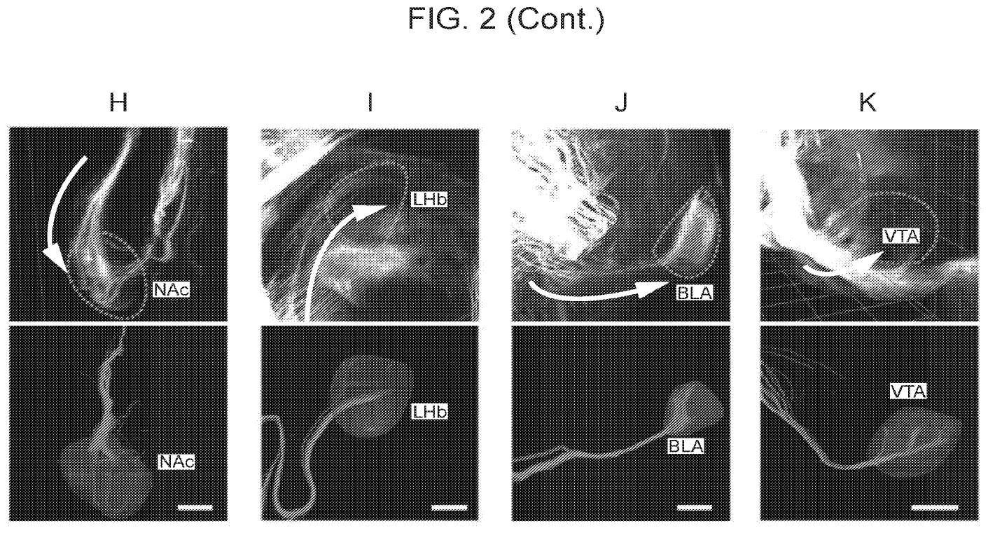

The three key features of this new approach were: 1) accelerated clarification through parallelized flow-assisted clearing crucial for large cohorts (FIGS. 1D-1G) independent of specialized equipment such as electrophoresis or perfusion chambers; 2)>90% cost reduction (also important for these large behavioral cohorts) using a new refractive index-matching process; and 3) optical properties such that the whole mouse brain can be imaged using a commercial light-sheet microscope (LSM) under a single field of view (FOV) and as a single stack (˜1200 steps across a ˜6.6 mm range) in less than 2 hours with single-cell resolution throughout the whole volume (this speed and simplicity is also critical for large behavioral cohorts; FIGS. 2C-2D). Raw data files from each brain are ˜12 GB in size and can be easily stored and directly analyzed on standard desktop workstations without the need for compression or stitching.

A hydrogel based on 1% acrylamide (1% acrylamide, 0.125% Bis, 4% PFA, 0.025% VA-044 initiator (w/v), in 1×PBS, Ref) was used for all CLARITY preparations. Mice were transcardially perfused with ice-cold 4% PFA. After perfusion, brains were post-fixed in 4% PFA overnight at 4° C. and then transferred to 1% hydrogel for 48 hours to allow monomer diffusion. The samples were degassed and polymerized (4-5 hours at 37° C.) in a 50 ml tube. The brains were removed from hydrogel and washed with 200 mM NaOH-Boric buffer (pH=8.5) containing 8% SDS for 6-12 hours to remove residual PFA and monomers. Brains could now be transferred to a flow-assisted clearing device using a temperature-control circulator or a simper combination of 50 ml tube and heated stirring plate (FIGS. 1D-1E). 100 mM Tris-Boric Buffer (pH=8.5) containing 8% SDS was used to accelerate the clearing (at 40° C.). Note that Tris-containing buffer should only be used after PFA is completely washed out as Tris has primary amide group that can potentially interact with PFA. With this setup, a whole mouse brain can be cleared in 12 days (with circulator, or 8 days for a hemisphere) or 16 days (with conical tube/stir bar). After clearing, the brain was washed in PBST (0.2% Triton-X100) for at least 24 hours at 37° C. to remove residual SDS. Brains were incubated in a refractive index matching solution (RapidClear, RI=1.45, Sunjin lab, “http://” followed by “www.sun” followed by “jinlab.” followed by “com/”) for 8 hours (up to 1 day) at 37° C. and then 6-8 hours at room temperature. After the RC incubation, the brains were ready for imaging.

### Histology

Mice were deeply anaesthetized and transcardially perfused with ice-cold 4% paraformaldehyde (PFA) in PBS (pH 7.4). Brains were fixed overnight in 4% PFA and then equilibrated in 30% sucrose in PBS. 40 μm thick coronal sections were cut on a freezing microtome and stored in cryoprotectant at 4° C. until processed for immunostaining. Free-floating sections were washed in PBS and then incubated for 30 min in 0.3% Triton X-100 (Tx100) and 3% normal donkey serum (NDS). Slices were incubated overnight with 3% NDS and primary antibodies including: rabbit anti-GABA (Sigma A2052 1:200), mouse anti-CaMKIIα (Abcam ab22609 1:200), chicken anti-GFP (Abcam ab13970 1:500), and rabbit anti-NPAS4 (gift from Michael Greenberg, 1:2500). Sections were then washed and incubated with secondary antibodies (Jackson Labs 1:1000) conjugated to donkey anti-rabbit Cy5, anti-mouse Cy3 and anti-chicken FITC for 3 hrs at room temperature. All NPAS4 staining was performed using a TSA-Cy5 amplification system (Perkin Elmer) according to the manufacturer's instructions. Following a 20 min incubation with DAPI (1:50,000) sections were washed and mounted on microscope slides with PVA-DABCO. Confocal fluorescence images were acquired on a Leica TCS SP5 scanning laser microscope using a 40×/1.25NA oil immersion objective. Serial stack images covering a depth of 20 μm through multiple sections were analyzed by an experimenter blind to treatment condition.

### QPCR and Gene Expression Analysis

For qPCR analysis, RNA was reverse transcribed using the ABI high capacity cDNA synthesis kit and used in quantitative PCR reactions containing SYBR-green fluorescent dye (ABI). Relative expression of mRNAs was determined after normalization with TBP levels using the ΔΔCt method.

### Cell Culture and In Vitro Activity Testing

Primary cultured hippocampal neurons were prepared from P0 Spague-Dawley rat pups and grown on glass coverslips as previously described. At 12 div cultures were transfected with 1 μg fosCh DNA using calcium phosphate. Immediately following the transfection procedure, cultures were returned to Neurobasal-A culture media (Invitrogen Carlsbad, Calif.) containing 1.25% FBS (Hyclone, Logan, Utah), 4% B-27 supplement (GIBCO, Grand Island, N.Y.), 2 mM Glutamax (GIBCO), and FUDR (2 mg/ml, Sigma, St. Louis, Mo.) to maintain high basal levels of intrinsic synaptic activity, or they were incubated in unsupplemented Neurobasal media that contained 1 μM tetrodotoxin (TTX), 25 μM 2-amino-5-phosphonopentanoic acid (APV) and 10 μM 2,3-dihydroxy-6-nitro-7-sulfamoyl-benzo[f]quinoxaline-2,3-dione (NBQX) to silence electrical activity. Cultures were stimulated for 30 min by exchanging the media with 60 mM isotonic KCl solution and then fixed with 4% PFA at indicated time points.

### In Vivo Optrode Recording

Simultaneous optical stimulation and extracellular electrical recording were performed in isofluorane-anesthetized mice. Optrodes consisted of a tungsten electrode (1 MΩ; 125 μm outer diameter) glued to an optical fiber (300 μm core diameter, 0.37 N.A.), with the tip of the electrode projecting beyond the fiber by 300-500 mm. The optical fiber was coupled to a 473 nm laser and 5 mW light measured at the fiber tip was delivered at 10 Hz (5 ms pulses). Signals were amplified and band-pass filtered (300 Hz low cut-off, 10 kHz high cut-off) before digitizing and recording to disk. pClamp 10 and a Digidata 1322A board were used to both collect data and generate light pulses through the fiber. The recorded signal was band pass filtered at 300 Hz low/5 kHz high (1800 Microelectrode AC Amplifier). Stereotaxic guidance was used for precise placement of the optrode, which was lowered through the dorsal-ventral axis of the mPFC by 50 μm increments. The percentage of sites yielding light-evoked action potential firing was determined.

### Real-Time Conditioned Place Preference

Behavioral experiments were performed 2 weeks after virus injections during the animals' dark (active) cycle. For induction of fosCh expression under appetitive or aversive conditions, mice received either i.p. injections of cocaine (15 mg/kg) or they underwent 20 random foot shocks (2 s, 0.5 mA, 2 shocks per minute on average). Mice were exposed to appetitive or aversive training twice a day over 5 consecutive days. Conditioned place preference (CPP) was conducted within 12-16 hours after the last appetitive or aversive training. The CPP apparatus consisted of a rectangular chamber with one side compartment measuring 23 cm×26 cm with multicolored walls, a central compartment measuring 23 cm×11 cm with white plexiglass walls, and another side compartment measuring 23 cm×26 cm with distinctive striped walls. Chamber wallpapers were selected such that mice did not display average baseline bias for a particular chamber, and any mouse with a strong initial preference for a chamber was excluded (more than 5 min difference spent in the side chambers during the baseline test). Automated video tracking software (BiObserve) was used to monitor mouse location over 3 consecutive 20 min blocks to assess place preference behavior before, during and after optogenetic stimulation of the fosCh labeled cells. During the light stimulation block, the laser was automatically triggered upon mouse entry into a pre-designated chamber (fully counterbalanced for side) to deliver 2 sec bursts of 10 Hz light pulses every 5 sec (5 ms pulses at 5 mW) for the duration that the mouse remained in the stimulation side. Data are expressed as fold-change in time spent in the light-paired side relative to the initial baseline preference.

### Statistics

Two-way ANOVAs were used to assess how gene expression or behavior was affected by other factors (e.g. neuronal activity, optogenetic manipulations). If a statistically significant effect was observed, post hoc testing with correction for multiple comparisons was performed using Tukey's multiple comparisons test. Unpaired t-tests were used for comparisons between two groups. Two-tailed tests were used throughout with α=0.05. Multiple comparisons were adjusted with the false discovery rate method. The experimenter was blinded to the experimental groups while running behavioral experiments and analyzing images. In all figure legends n refers to biological replicates.

### Example 1: Resolving mPFC Populations and Projections Activated by Appetitive or Aversive Experience

Similarity in activation pattern by appetitive and aversive experiences has been reported in individually-selected brain regions (verified broadly, though not in all regions, by the brainwide analysis conducted here). A falsifiable hypothesis arising from these observations would be that the same neuron type distribution was recruited by the two stimuli, for example reflecting neurons in each region reporting on arousal state due to the salience of the experience. In mPFC, other existing literature alone does not support or falsify this hypothesis, though mPFC is associated with specific reward and aversion processes (including cocaine-conditioned place preference on the one hand, as well as fear and anxiety behaviors on the other), in addition to more general functions potentially relevant to the single-population hypothesis (including attention, salience- and novelty-detection, and working memory). The region-specific differential activation detected by the brain-wide analysis reported here may open the door to considering a distinct hypothesis at least for some circuits—that appetitive and aversive experience recruit distinct neuronal populations. Connectivity is one of the most important features that might resolve principal cell population types involved in such distinct processes, but this feature has been difficult to explore in a brain-wide fashion while remaining linked (at the single-cell level) to function during behavior.

A very strongly-expressed activity-dependent cell-filling label (unlike traditional nuclear c-fos immunostaining or typical transiently or transgenically-expressed fluorophores) in principle might allow for acquisition of this crucial wiring information as well from the same experimental subjects, provided that axon tracts of labeled and filled neurons could be robustly imaged and quantified in this context. With the goal of building such a probe, a novel CLARITY-optimized axonal-filling enhanced fluorescent protein, engineered in part by inserting the 3′ UTR of neuritin (NRN) RNA at the C-terminus of EYFP was developed. It was found that this DNA construct could be readily packaged into high-titer adeno-associated virus (AAV) capsids that indeed enabled focal injection-defined projection labeling in CLARITY; for example, efferent mPFC projections could be readily followed throughout the entire adult mouse brain after a single stereotaxic injection (FIGS. 1A-1B). Visualizing axonal tracks in 3D revealed key topographical features that were difficult, if not impossible, to detect in thin 2D sections (FIG. 1A, FIG. 2A); for example, a prominent axon bundle traveling from mPFC to ventral medial thalamus was observed to carry out a sharp U-turn near the VTA (FIGS. 1C-1D), a potentially important feature that has not been described in existing atlases (FIG. 2B).

FIG. 1: CLARITY enables brain-wide origin/target-defined projection mapping. (FIG. 1A) 2D orthogonal views (horizontal, sagittal and coronal) of a mouse brain. Insert shows schematic for location of viral injection. Orientations: D: dorsal, V: ventral, A: anterior, P: posterior, L: lateral, M: medial. (FIG. 1B) Three-dimensional rendering of CLARITY hemisphere, visualizing outgoing mPFC projections (imaged by 2× objective at 0.8× zoom with a single FOV, step size: 4 μm, 1000 steps). (FIG. 1C) 3D visualization of the axonal bundle projecting from mPFC to VM, showing tracts turning near the VTA (indicated by arrows). (FIG. 1D) Visualizing the same projection in (FIG. 1C) with sparse labeling (using lower-titer virus). (FIG. 1E) Raw image from a CLARITY volume. Orange: user-defined “seed region” so that only the fibers passing this region were tracked. (FIG. 1F) Streamlines reconstructed from (FIG. 1E), using structural tensor-based tractography. Note that fibers in the CLARITY image that did not pass the user-defined seed region were excluded in the reconstruction (indicated by the magenta arrows). (FIG. 1G) Reconstructed brain-wide streamlines from CLARITY image in (FIG. 1B). The streamlines are color-coded for orientation. A-P: red; D-V, green; L-M, blue. (FIG. 1H) Representative computational isolation of mPFC fibers that project to VTA (yellow) or BLA (green). All scale bars: 500 μm.

FIG. 2: CLARITY enables brain-wide origin/target-defined projection mapping. (FIG. 2A) 2D coronal sections (50 μm max-projection) at the indicated locations (relative to bregma). Scale bar: 500 μm. (FIG. 2B) A snapshot of putative mPFC to VM (highlight in green) projection paths (shown as red streamlines) from the Allen Brain mouse connectivity atlas (“http://” followed by “connectivity.brain-map” followed by “.org/”). Scale bar: 1 mm. (FIG. 2C-2F) Representative intermediate steps of reconstructing axonal projection to streamlines using structural tensor based CLARITY tractography. (FIG. 2C) Raw CLARITY image, showing outgoing mPFC projections (EYFP). (FIG. 2D) Image intensity gradient amplitude, computed by convolving the 3-dimensional CLARITY image volume with three 3-dimensional 1^{st} order derivative of Gaussian functions (σdog=1 voxel/6 μm) along each of the x, y and z axes. (FIG. 2E) Color-coded principal fiber orientations (A-P: red; D-V, green; L-M, blue), estimated as the tertiary eigenvectors of computed structure tensors (σd=1 voxel/6 μm, σdog=1 voxel/6 μm). For better visualization, the color brightness was weighted by the raw CLARITY image intensity. Scale bars: 100 μm. (FIG. 2F) A zoomed-in region of (FIG. 2E) showing the principal fiber orientations as color-coded vector fields overlaid on raw CLARITY image. The vectors are color-coded for their orientation. Scale bar: 6 μm. (FIG. 2G) Correlation between the diameter of each axonal bundle and the number of streamlines representing that specific bundle. The diameter was determined at the cross-sections of each bundle. The numbers of passing streamlines are also measured at the same cross-sections. n=15, Pearson correlation, r²=0.96, P<0.0001. (FIGS. 2H-2K) Representative reconstructions of axonal projections (outgoing projections from mPFC) in various target regions: Nac (FIG. 2H), LHb (FIG. 2I), BLA (FIG. 2J) and VTA (FIG. 2K). Top row: CLARITY images; bottom row: reconstructed streamlines ending in the indicated 3D regions.

A method to compute 3D structure tensors from CLARITY images for tractography was developed in order to quantify tracts across large behavioral cohorts (FIGS. 2C-2F). Faithful reconstruction of calculated streamlines was achieved (using tools adapted from magnetic resonance image analysis for diffusion tractography); these streamlines mapped onto fibers from CLARITY images (FIGS. 1E-1F) and importantly, the streamline count in each bundle tightly correlated with the ground-truth physical diameter of the axonal bundles (FIG. 2G). Using this method, whole brain projections (originating from mPFC AAV injections) were reconstructed based on 3D CLARITY images (FIG. 1G); connectivity between a seed region (here defined by stereotaxic injection site) and any specified downstream target such as BLA or VTA, could be readily visualized and assessed by counting streamlines (FIG. 1H, FIGS. 2H-2K).

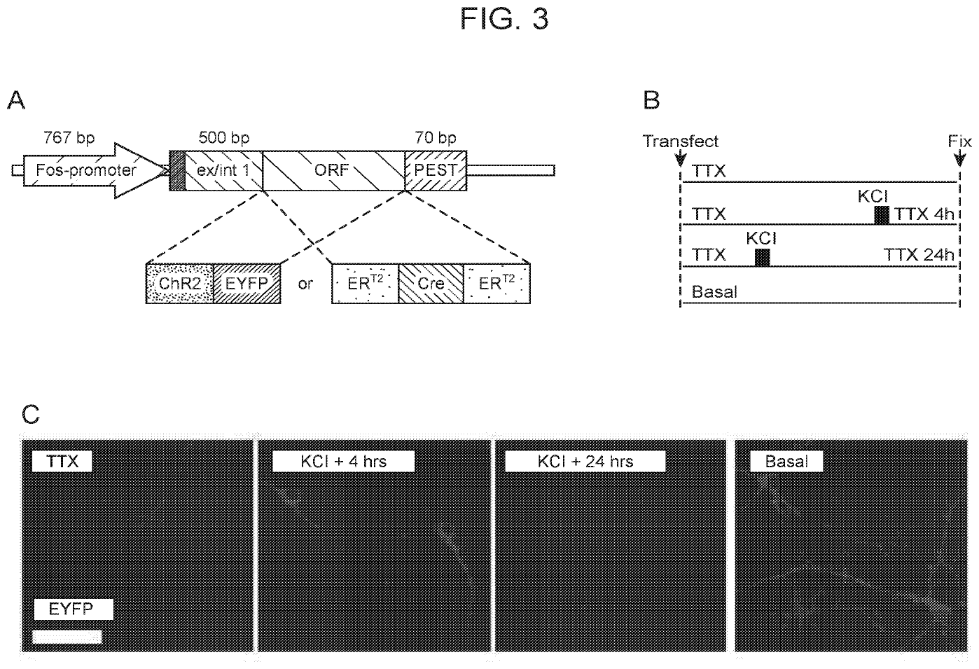

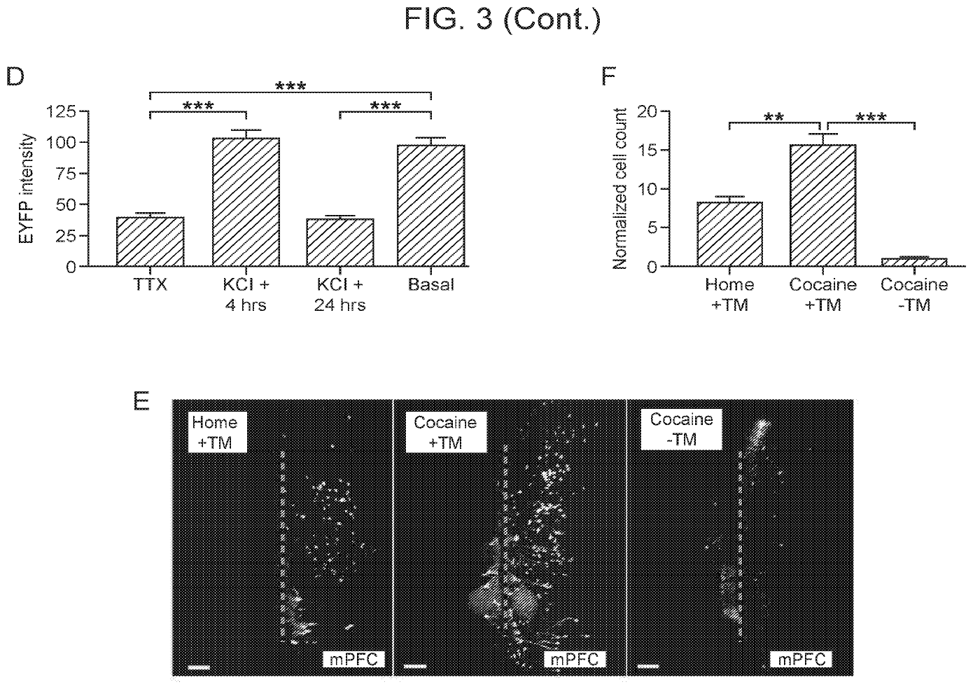

To integrate this new capability, with the needed additional capability of projection-labeling in cells defined by their use during behavioral experience, a viral CreER/4TM strategy was developed to translate time-locked activity to sustained transgene expression (it was found that typical transgenic fluorophore expression driven by an activity-dependent promoter was insufficiently strong for tractography). Therefore, a c-Fos promoter combining minimal promoter and regulatory elements in intron-1 was engineered (FIG. 3A) that was small enough to be packaged into AAV particles and specific enough to capture elevations in neuronal activity (FIGS. 3B-3D). A destabilized ER-Cre-ER-PEST cassette was also inserted under this promoter; when injected into the Ai14 reporter mouse, this viral CreER/4TM system reliably enabled activity- and tamoxifen-dependent cell body and projection labeling (FIGS. 3E-3F).

FIG. 3: Distinct projection targets of cocaine and shock-activated mPFC populations. (FIG. 3A) Construction strategy. An expression cassette was inserted immediately after intron 1 of the c-fos gene. Either ChR2-EFYP (cFos-ChR2-EYFP, termed fosCh) or ER^{T2}-Cre-ER^{T2} fusion was inserted, followed by a 70 bp PEST sequence to promote construct degradation (to further enhance specificity). (FIG. 3B) Schematic to illustrate treatment of cultured hippocampal neurons following transfection of c-Fos-ChR2-EYFP. Neurons were electrically silenced with TTX, APV and NBQX; fosCh expression was compared to expression levels in “basal” (spontaneously synaptically active, but not otherwise stimulated or silenced) cultures. Following a 30 min depolarizing stimulus (60 mM KCl) the TTX/APV/NBQX solution was replaced and groups were fixed at the indicated time points. (FIG. 3C) Representative images showing fosCh expression of cultured hippocampal neurons for each of the treatment groups. Scale bar: 25 μm. (FIG. 3D) Quantification of mean pixel intensity of EYFP expression for conditions represented in c, n=39-59 cells per group, F_{3, 205}=37.20, ***P<0.001, ANOVA followed by Tukey's multiple comparison test. (FIGS. 3E-3F) AAV-cFos-ER^{T2}-Cre-ER^{T2}-PEST was injected into the mPFC of Ai14 Cre-reporter mice. The mice were divided into three groups (n=5 per group): home cage with 4TM, cocaine-injected with 4TM and cocaine-injected without 4TM. (FIG. 3E) Representative images showing 4TM-dependent and activity-dependent labeling of mPFC neurons (tdTomato+), scale bar: 100 μm. (FIG. 3F) Quantification tdTomato+ mPFC cells in three groups (normalized to the No-4TM group). **P<0.01, ***P<0.001, unpaired t-test. Error bars, mean±s.e.m.

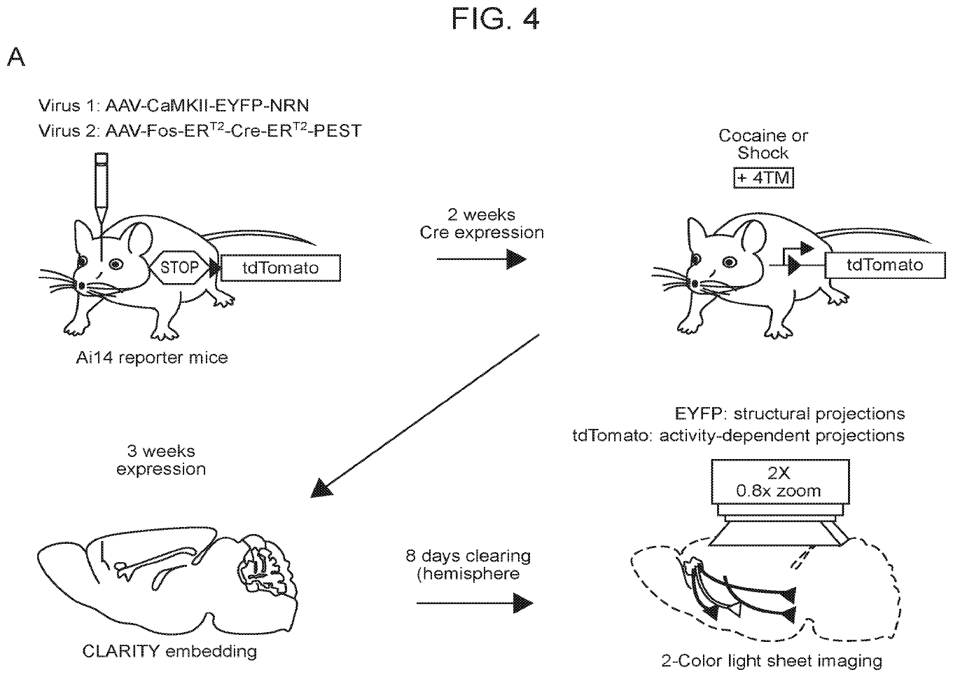

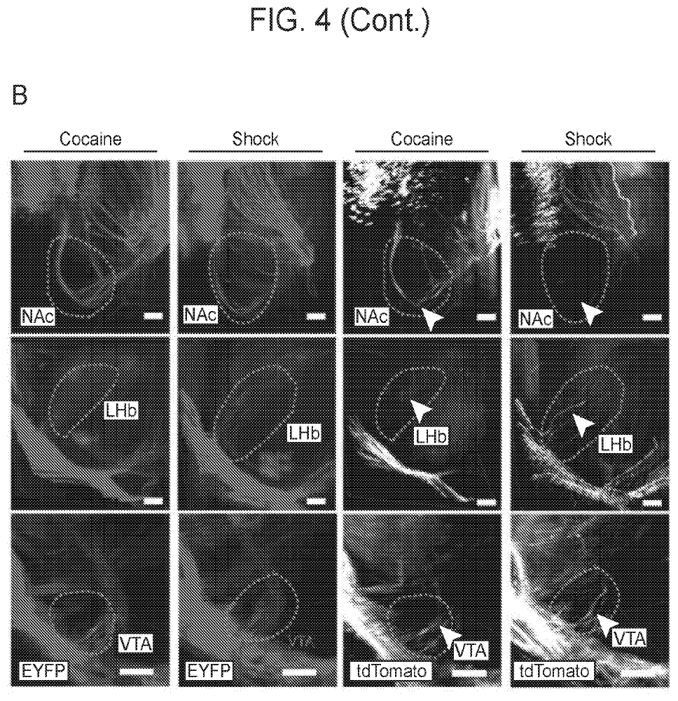

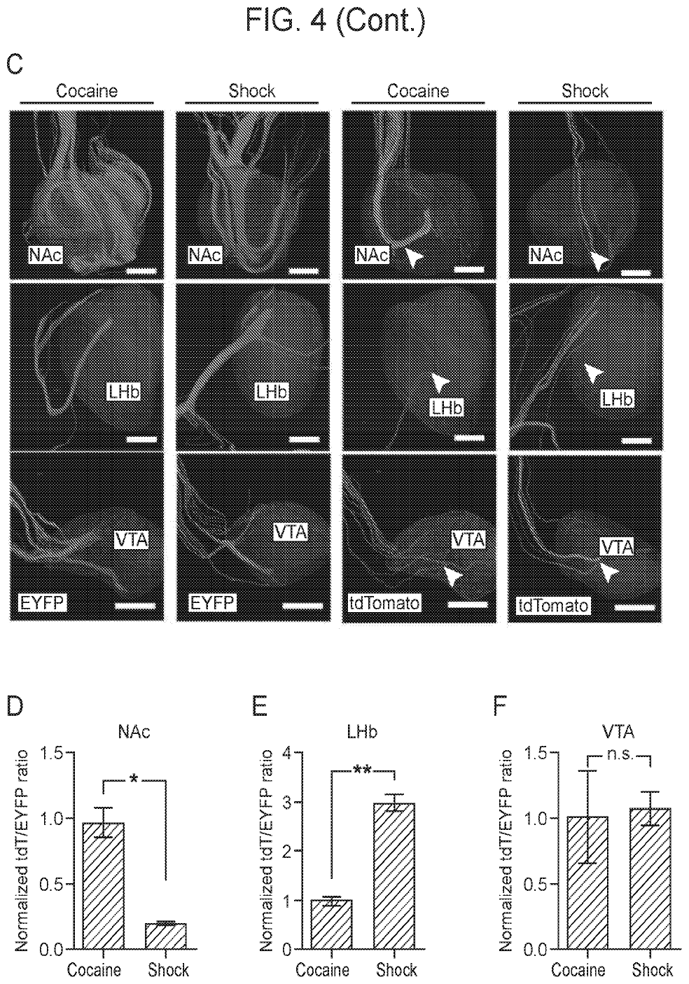

A final essential feature (for behavioral cohort-wide quantitative activity-dependent projection mapping) was enablement of normalization on an individual-subject level to the absolute tract labeling strength independent of activity; this normalization is in principle crucial in a virus-based approach to control for variation in injection efficacy. This feature (FIG. 4A) was enabled by building in simultaneous two-color activity-independent (structural, EYFP) labeling and activity-dependent (tdTomato) labeling of projections from the same injection site. Dual-color quantification of projections across the intact brain to multiple downstream regions is then achieved by counting the number of streamlines ending in these regions, and the activity-dependence is corrected for anatomical and injection variability from the red/green streamline ratio. This quantification of projection use across the brain from behaviorally-defined neuronal populations is (for brevity) termed here CLARITY-based Activity Projection Tracking upon Recombination, or CAPTURE (FIG. 4A).

FIG. 4: Distinct projection targets of cocaine and shock-activated mPFC populations. (FIG. 4A) Summary of CAPTURE workflow (described in text). (FIG. 4B) Representative CLARITY images of the structural projections (green: EYFP) and activity-dependent projections (white: tdTomato) from cocaine- and shock-labeled mice in Nac (top row), LHb (middle row) and VTA (bottom row). Arrowheads indicate axon bundles terminating in the circled region. Scale bar: 200 μm. (FIG. 4C) Reconstructed streamlines from (FIG. 4B), showing streamlines terminating in the 3D brain regions (purple). Green streamlines: reconstructed from EYFP fibers; red streamlines: reconstructed from tdTomato fibers. Scale bars: 200 μm. (FIGS. 4D-4F) Quantification of projection intensity from cocaine- and shock-activated mPFC populations in three regions. Behavior-specific projection intensity was quantified using the ratio between red and green fibers (i.e. the number of red streamlines divided by the number of green streamlines) terminating in indicated 3D regions (Nac, LHb and VTA; n=6 per group; ns, P>0.05, *P<0.05, **P<0.01, unpaired t-test). Error bars, mean±s.e.m.

### Example 2: Distinct Projection Patterns Among Behavioral Experience-Defined mPFC Populations

CAPTURE was applied to quantify projections from cocaine- and shock-recruited mPFC populations. Two groups of Ai14 reporter mice were co-injected with CaMKIIα-EYFP-NRN and cFos-ER-Cre-ER-PEST AAVs, and subjected to 4TM-mediated cocaine- and shock-labeling. With CAPTURE, projections from all CaMKIIα (principally excitatory glutamatergic) neurons are labeled with EYFP and projections from behaviorally-recruited populations are labeled with tdTomato. Importantly, EYFP fibers in the Nac, BLA and VTA were found to be indistinguishable between the cocaine- and shock-labeled animals, indicating minimal variation in viral injection, transduction, and expression between the two groups (FIG. 4B).

In the very same animals, significantly more projections from behaviorally-active mPFC neurons were observed targeting the Nac in cocaine-exposed animals compared to shock-exposed animals. Conversely, significantly more behaviorally-active mPFC fibers to the LHb in shock-exposed animals were observed (FIGS. 4C-4F). No significant difference in red/green (activity/structure) ratio was observed between the two groups in mPFC projections to the VTA, revealing no detectable systematic difference in efficiency or targeting of viral anatomical labeling. The cocaine-activated mPFC population thus preferentially projects to the Nac whereas the shock-activated population projects more strongly to LHb, revealing that the populations of neurons that are recruited in mPFC by distinct-valence behavioral experience are not simply different in terms of the patterns of input that they happen to receive, but represent anatomically distinct cell populations in terms of projection pattern across the brain.

### Example 3: Cocaine- and Shock-Activated Populations Control Appetitive and Aversive Behaviors

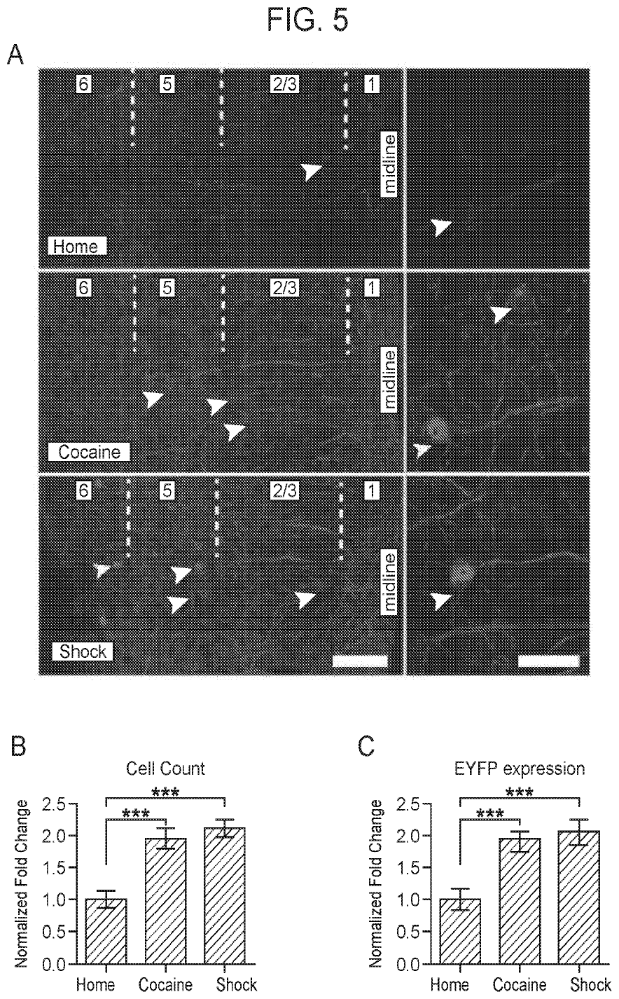

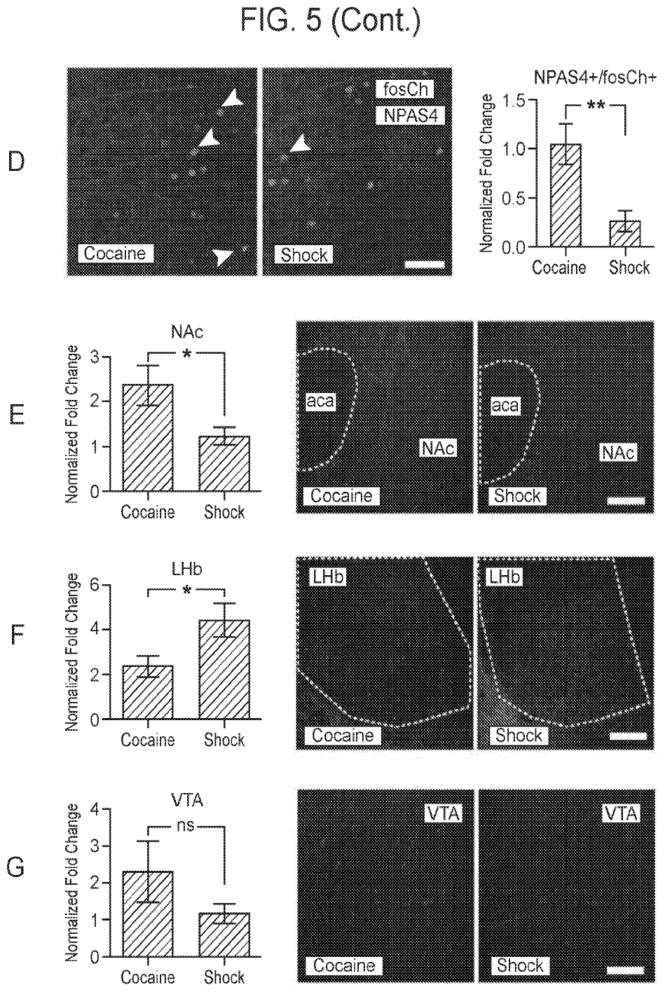

Having established that mPFC neuronal populations recruited under the appetitive and aversive conditions were separable by gene expression signature and long-range connectivity measures, it was next tested if electrical activity in these two behavioral activity-defined populations had distinct positive or negative conditioning valence for the same animals that had experienced the stimulus, assessed by causal impact on behavior during the place preference task. A codon-optimized channelrhodopsin tagged with EYFP (ChR2-EYFP) under the control of the AAV-cFos backbone (termed fosCh; FIG. 3A) was used, and stereotaxically injected fosCh into mPFC. For 5 consecutive days these animals were exposed to daily cocaine administration or foot shock behavioral experience. After exposure, a significant increase in the number of fosCh-labeled cells and mean EYFP expression level compared to controls was observed (FIGS. 5A-5C).

FIG. 5: Use of fosCh for targeting cocaine- and shock-activated mPFC populations. (FIG. 5A) Representative images showing fosCh expression in mPFC following the indicated behaviors. Left, images visualizing lamina across the cortical depth (midline is on the right). Arrowheads indicate fosCh positive neurons. Scale bars: 100 μm. Right, high-magnification images of individual fosCh neurons. Scale bars: 25 μm. (FIG. 5B) Fold change in fosCh cell numbers (normalized to home cage level). (FIG. 5C) Fold change in mean EYFP fluorescence intensity. n=11-14 per group, ***P<0.001, unpaired t-test. (FIG. 5D) Representative images and quantification of fosCh and NPAS4+ cells. Arrowheads indicate double-positive cells. n=5 per group, **P<0.01, unpaired t-test. (FIGS. 5E-5G) Left: Comparing density of fosCh projections for cocaine and shock groups. Right: representative images showing the density of fosCh projections in indicated regions. aca: anterior part of anterior commissure. Scale bars: 100 μm. n=11-14 per group, *P<0.05, unpaired t-test. Error bars, mean±s.e.m.

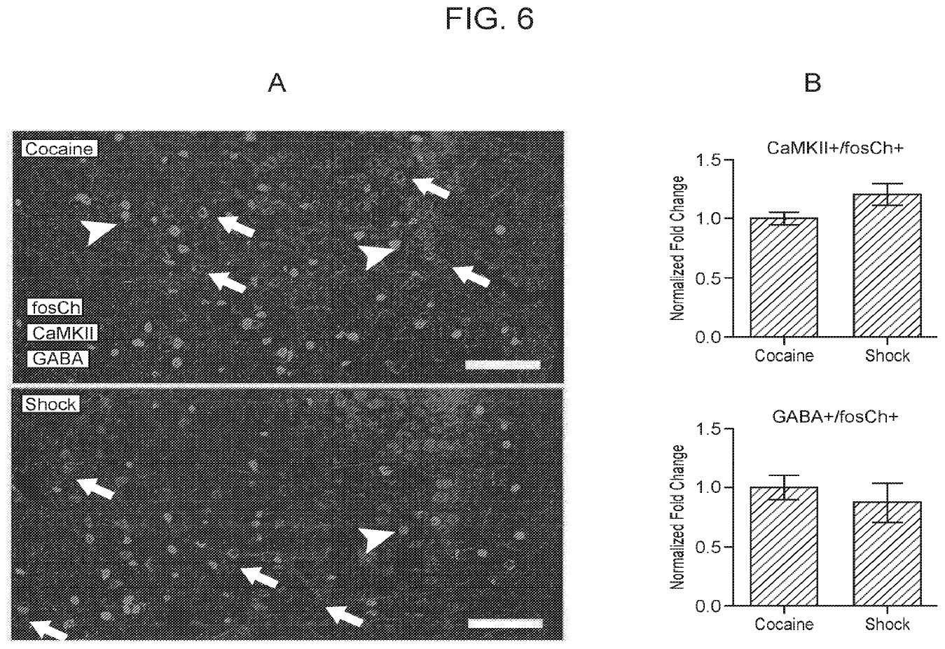

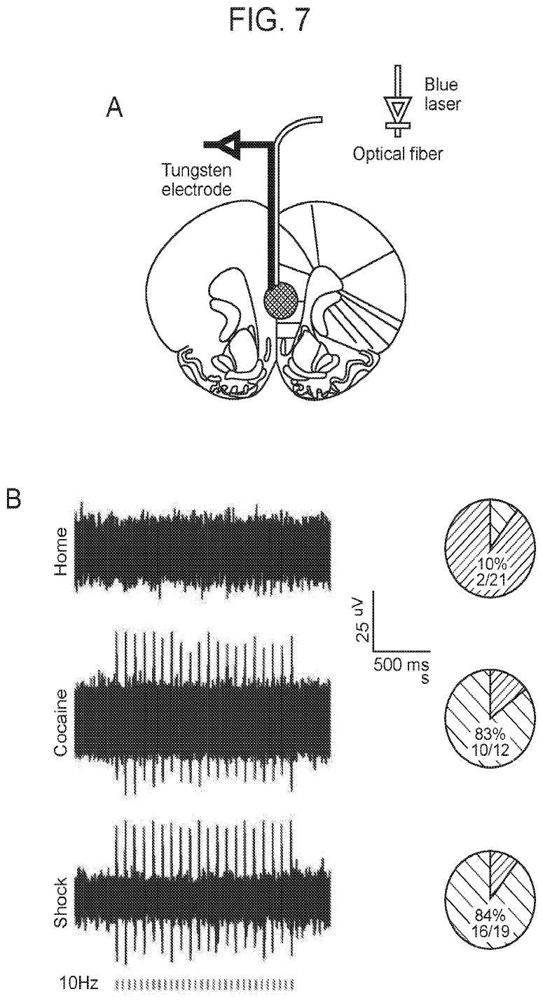

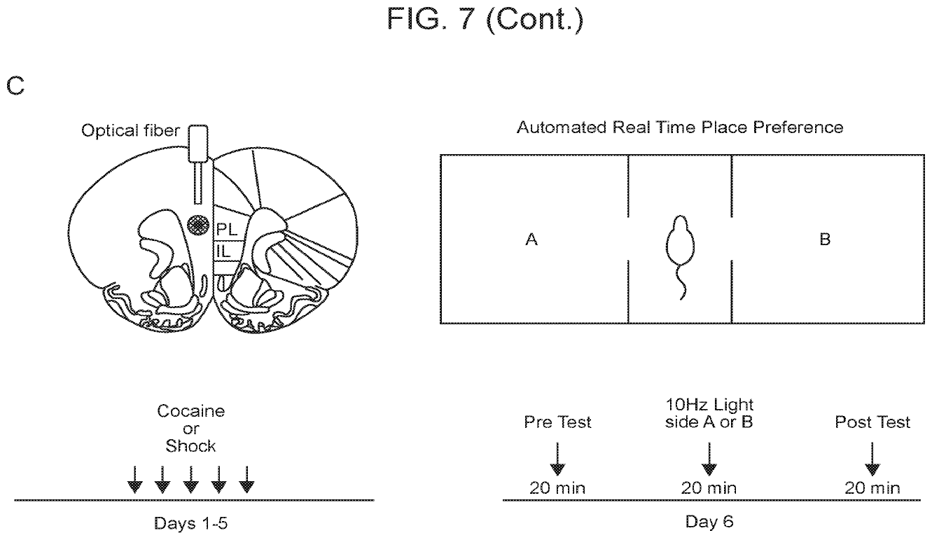

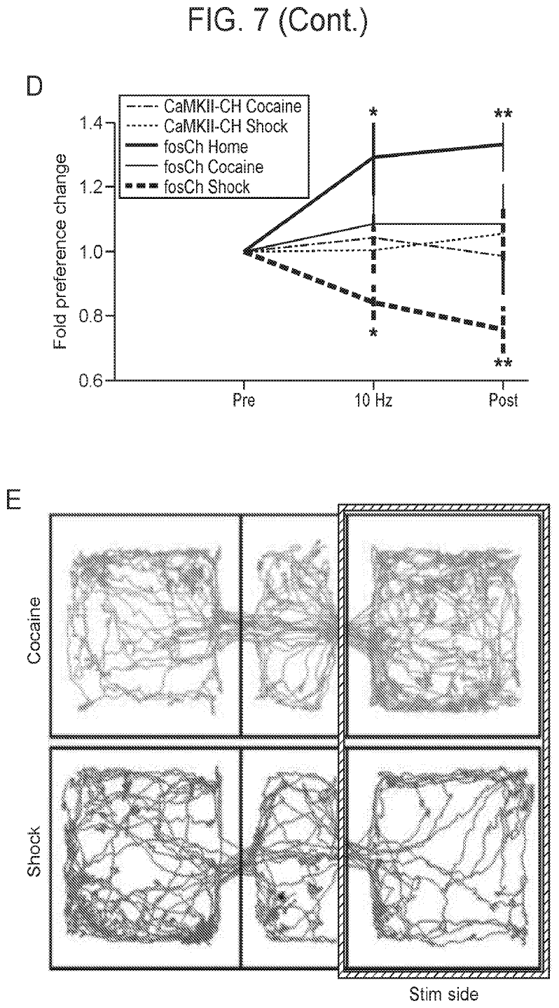

Npas4 expression was first quantified in cocaine- and shock-labeled fosCh cells, and it was hypothesized that cocaine-labeled fosCh cells would exhibit significantly higher Npas4 expression compared with shock-labeled fosCh cells. This was indeed the case (FIG. 5D); importantly, expression of general excitatory or inhibitory neuronal markers did not differ between those two populations (FIGS. 6A-6B). Moreover, consistent with CAPTURE findings, cocaine-labeled fosCh cells were found to project strongly to Nac, while the LHb contained significantly denser EYFP fibers arising from the shock-labeled fosCh cells (FIGS. 5E-5G). Crucially, this method of targeting was sufficiently potent to enable optical control over the resulting sparsely-distributed neuronal subsets; fosCh-labeled cells displayed robust light-evoked firing assessed by in vivo electrophysiological recording (FIGS. 7A-7B). Together, these data demonstrated resolution with the fosCh strategy of the same pattern that had been characterized molecularly and anatomically, and enabled the final test of whether these neuronal subsets were capable of differentially controlling behavior.

FIG. 6: Use of fosCh for targeting cocaine- and shock-activated mPFC populations. (FIG. 6A) Representative confocal images showing fosCh expression in mPFC sections co-labeled with anti-GABA, and anti-CaMKIIα antibodies as indicated. White arrows indicate fosCh+/CaMKIIα+ neurons. Yellow arrowheads indicate fosCh+/GABAα+ neurons. (FIG. 6B) Quantification revealed no significant difference in the number of CaMKIIα-positive (left) and GABA-positive (right) fosCh cells for cocaine and shock groups. n=10-14 mice per group. Error bars, mean±s.e.m.

FIG. 7: Differential behavioral influence of cocaine- and shock-activated mPFC populations. (FIG. 7A) Schematic to illustrate the placement of the recording electrode and optical fiber for in vivo recording experiments. The optrode was lowered in 100 μm steps along the dorsal-ventral axis of mPFC. (FIG. 7B) Left, representative extracellular recordings showing neural response to a 10 Hz light train (5 ms pulses for 2 sec, every 5 sec, 5 mW 473 nm blue light, indicated by blue bars). Right, pie charts indicate percentage of recording sites showing light-evoked action potential firing for the home cage (grey), cocaine (red), and shock (blue) groups. (FIG. 7C) Schematic shows the location of the optical fiber positioned above the injection site in green. After 5 days of training, mice were tested by real time place preference test which consisted of 3 consecutive 20-minute trials. (FIG. 7D) Behavioral results plotted as fold-change in preference for the light stimulated side (normalized by initial baseline preference) across each of the trials. n=10-14 per group, *P<0.05, **P<0.01, ANOVA followed by Tukey's multiple comparison test. Error bars, mean±s.e.m. (FIG. 7E) Movement tracking data from representative cocaine- and shock-labeled animals during the light stimulation trial.

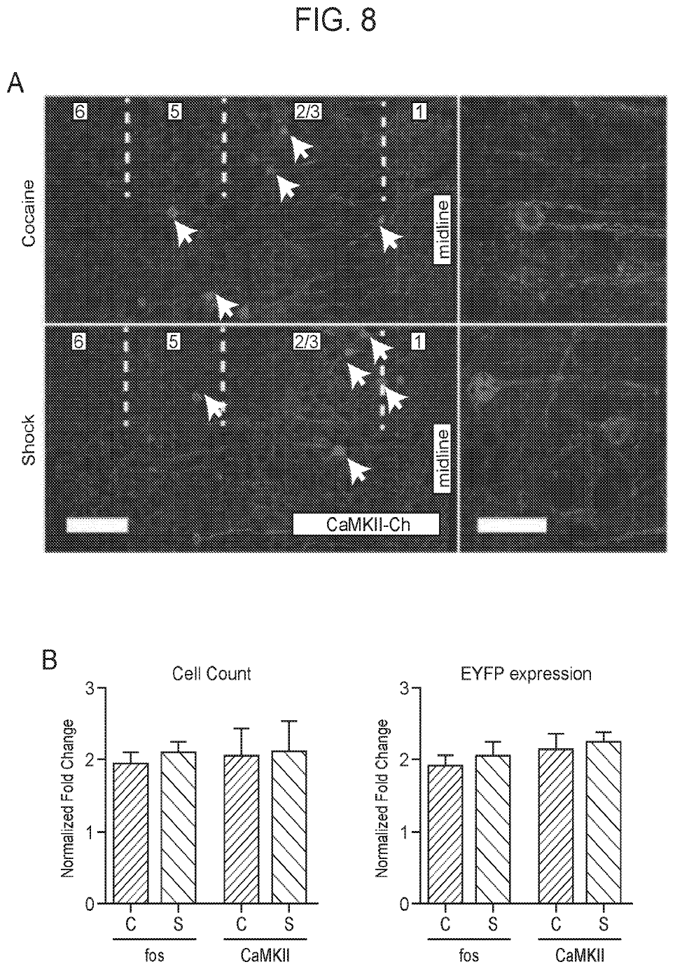

To address this question, the real-time place preference paradigm in which 10 Hz light pulse trains were automatically triggered upon entry into one side of a behavioral chamber was employed. Mouse behavior was monitored over 3 consecutive 20-minute trials to quantify place preference, before, during, and after light delivery for reactivation of fosCh-defined neuronal ensembles (FIG. 7C). Additional experimental arms in which expression of ChR2 was driven by the CaMKIIα promoter without link to prior activity was included, to control for the possibility that behavior could be biased by randomly-labeled neurons; in this control arm, virus was titered to target a similar number of mPFC neurons and matched to fosCh expression levels following cocaine or shock exposure (FIGS. 8A-8B). Optogenetic stimulation of these non-activity-specific neuronal populations did not influence place preference, nor were homecage-recruited fosCh-population animals observed to exhibit preference or aversion for the chamber in which the cells were optically activated. Remarkably, however, reactivation of the shock or cocaine-defined fosCh populations induced significant (and opposite-direction) shifts in place preference, with cocaine-exposed mice demonstrating preference, and shock-exposed mice demonstrating aversion, for the photostimulation-paired side (FIGS. 7D-7E; mean preference change at post-test for cocaine: 1.3×+/−0.1, Wilcoxon P=0.0006; for shock: 0.8×+/−0.1, Wilcoxon P=0.002). These data reveal that the activity-defined mPFC neural populations differ not only anatomically and molecularly, but also in functional impact in modulating behavior.

FIG. 8: Differential behavioral influence of cocaine- and shock-activated mPFC populations. (FIG. 8A) Representative images showing mPFC expression of CaMKIIα-ChR2 control conditions. Left, two 40× images were stitched together to visualize all cortical lamina. Scale bar=100 μm. Right, high magnification images of individual CaMKIIα-ChR2 neurons. Scale bar=25 μm. (FIG. 8B) Quantification revealed no significant difference in the number of labeled cells (left) or level of EFYP expression (right) between CaMKIIα-ChR2 and fosCh conditions. n=13 mice per group. Error bars, mean±s.e.m.

### Example 4: An Activity-Dependent Regulatory Region and Related Constructs

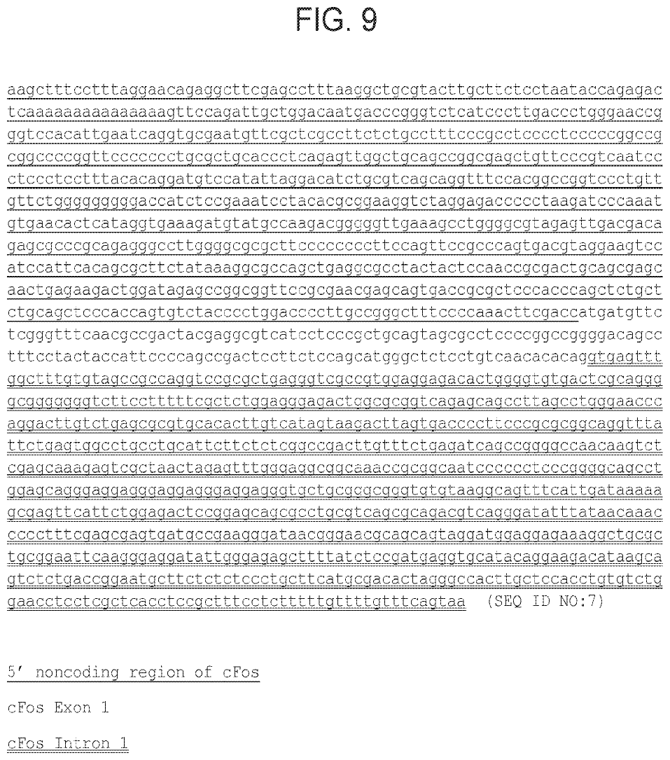

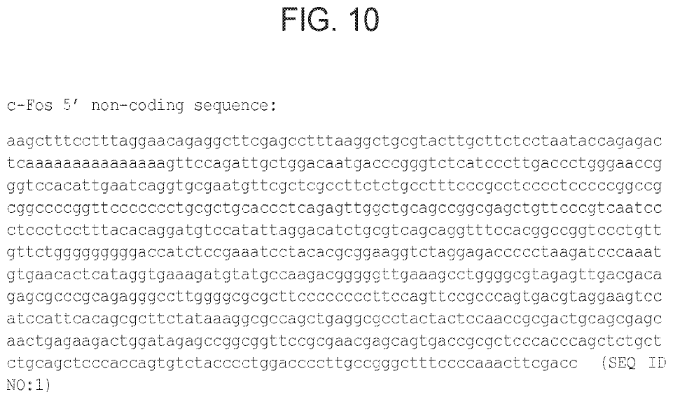

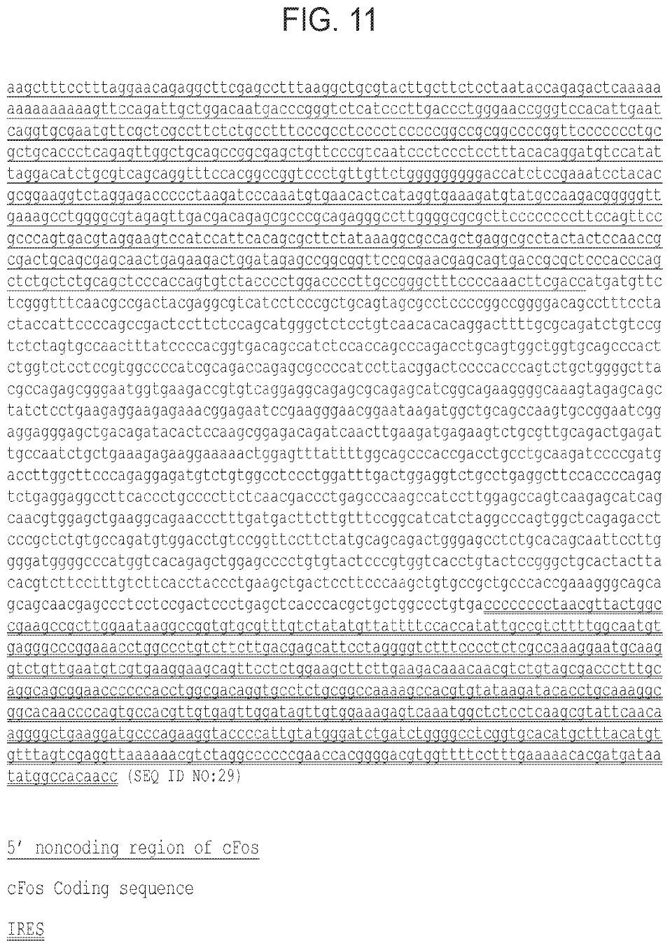

The activity-dependent expression of a reporter construct driven by a regulatory region containing 761 bp of 5′ mouse c-Fos non-coding sequence, mouse c-Fos exon 1 and mouse c-Fos intron 1, as depicted in FIG. 9 (SEQ ID NO:7) was compared to: (1) the activity-dependent expression of the same reporter driven by the c-Fos 5′-non-coding sequence depicted in FIG. 10 (SEQ ID NO:1) and (2) the activity-dependent expression of the same reporter driven by the entire c-Fos gene followed by an IRES as depicted in FIG. 11 (SEQ ID NO:29).

The activity-dependent expression from the reporter driven by the regulatory region containing 761 bp of 5′ mouse c-Fos non-coding sequence, mouse c-Fos exon 1 and mouse c-Fos intron 1, as depicted in FIG. 9 (SEQ ID NO:7) was found to be best for high, non-leaky expression. In comparison, alternative construct (1), the reporter driven by the c-Fos 5′-non-coding sequence only, was found to be extremely leaky and non-specific. In addition, the expression from alternative construct (2), the reporter driven by the entire c-Fos gene followed by an IRES, was found to be poor.

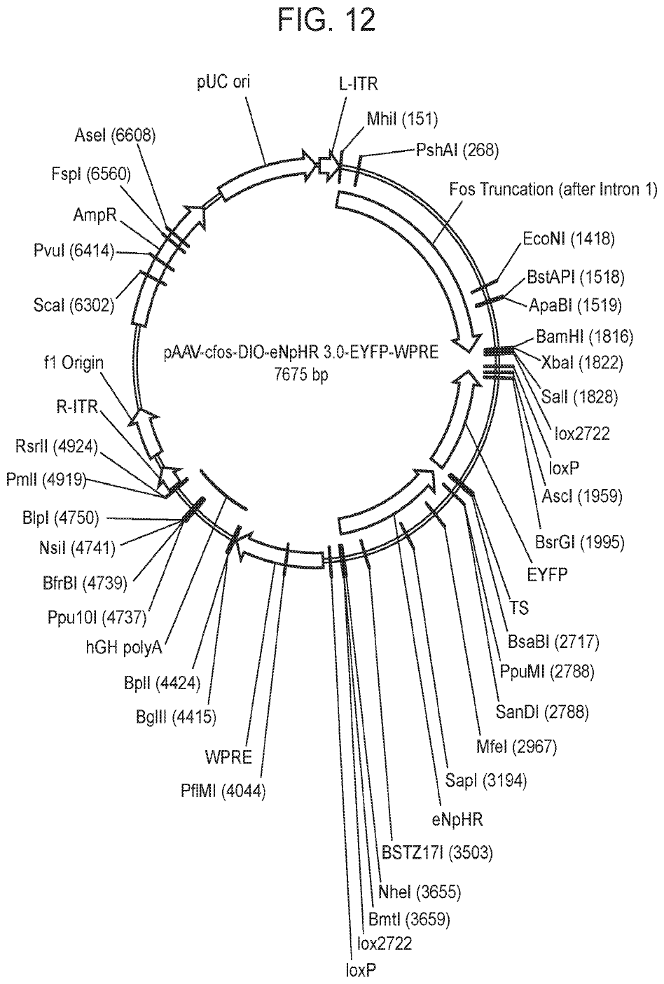

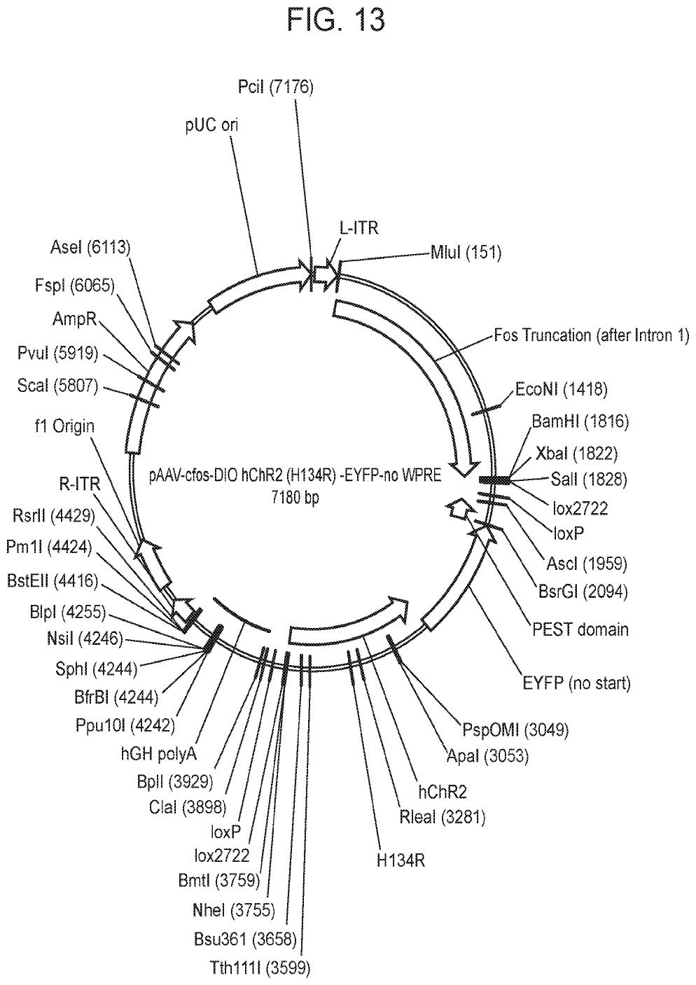

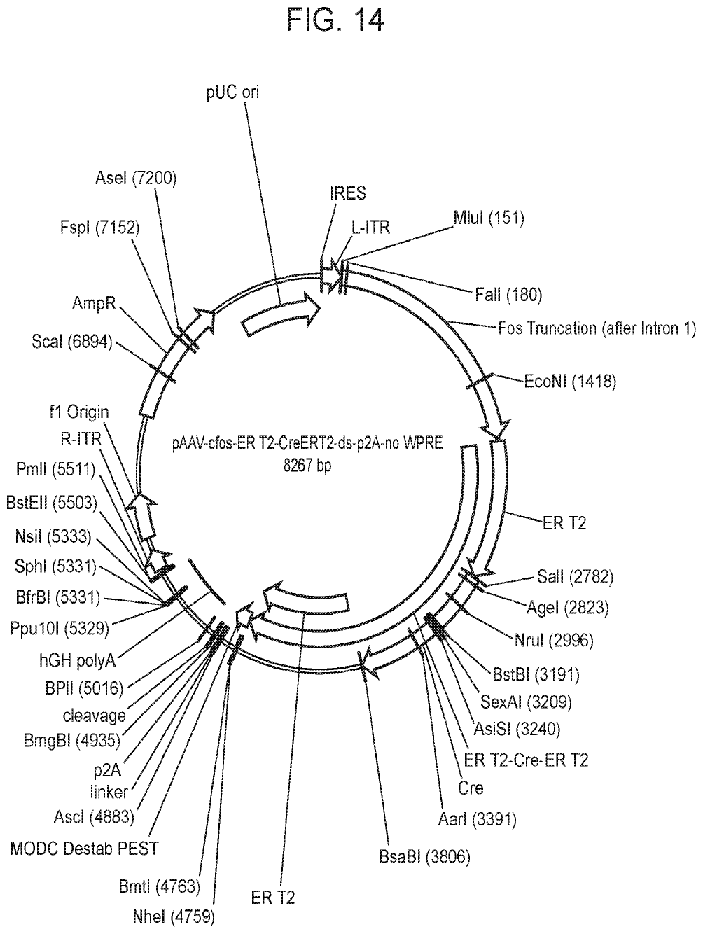

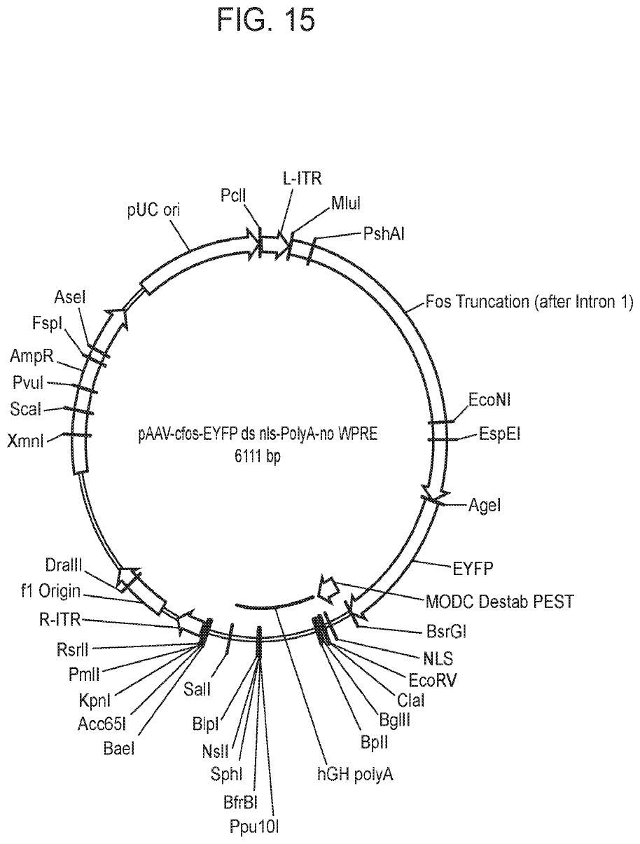

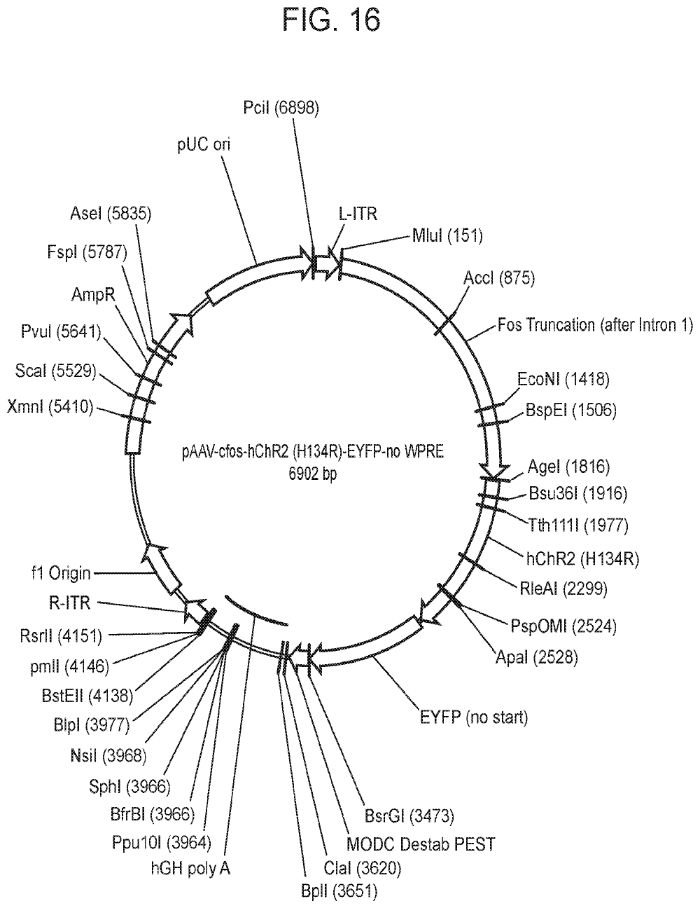

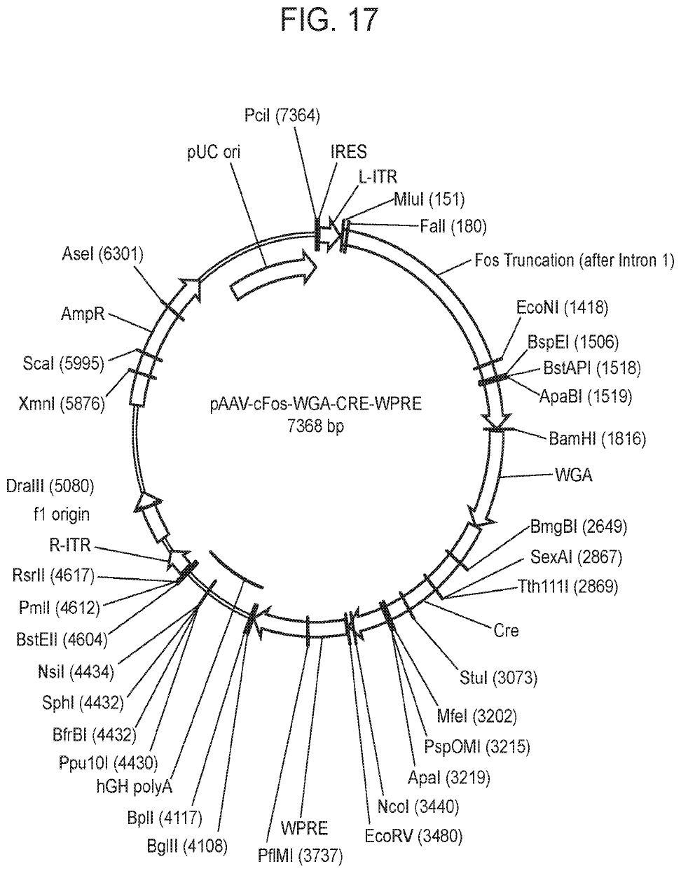

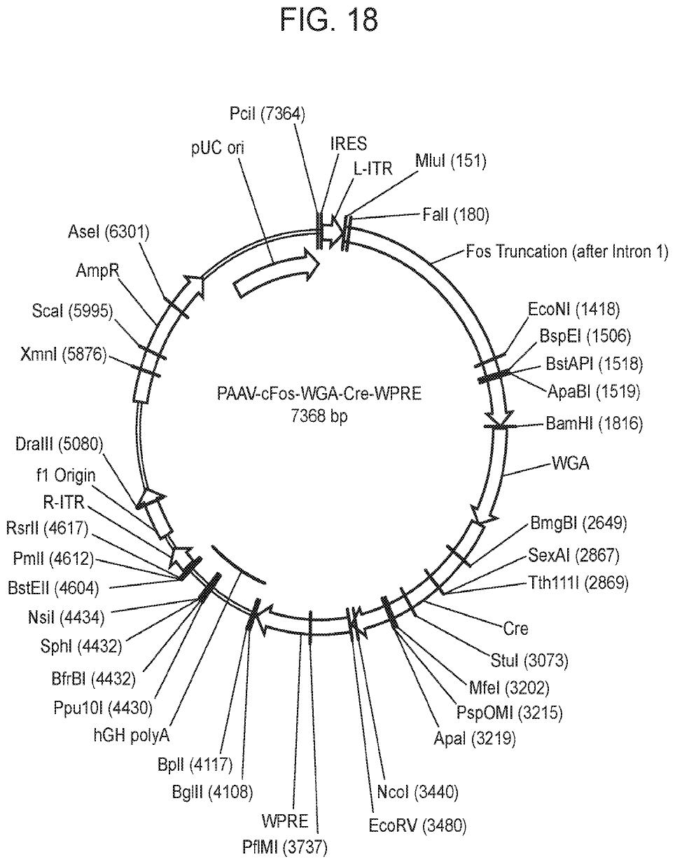

Accordingly, the regulatory sequence containing the 5′-non-coding sequence, first exon and first intron was found to have the best expression control parameters as compared to the alternative regulatory constructs tested. Therefore, various expression constructs were created using this regulatory sequence including but not limited to e.g., those depicted in FIG. 12 (pAAV-cFos-DIO-eNpHR 3.0-eYFP-PEST), FIG. 13 (pAAV-cFos-D10-hChR2(H134R)-eYFP-PEST), FIG. 14 (pAAV-cFos-ER-CreT-ER-ds-p2A), FIG. 15 (pAAV-cFos-eYFP-PEST), FIG. 16 (pAAV-cFos-hChR2(H134R)-eYFP-PEST), FIG. 17 (pAAV-cFos-WGA-Cre) and FIG. 18 (pAAV-cFos-WGA-Cre-WPRE).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An expression vector comprising, an activity-dependent expression cassette comprising:
(a) a regulatory sequence comprising a c-Fos 5′-non-coding region and a c-Fos first intron sequence; and
(b) a polypeptide coding sequence operably linked to the regulatory sequence, wherein the polypeptide encoded by the polypeptide coding sequence is expressed from the expression cassette upon activity-dependent activation of the regulatory sequence.

2. The expression vector of claim 1, wherein the vector is a viral vector.

3. The expression vector of claim 2, wherein the viral vector is a recombinant adeno-associated virus (AAV) vector.

4. The expression vector of any of claims 1-3, wherein the regulatory sequence is a mammalian c-fos regulatory sequence comprising a mammalian c-Fos 5′-non-coding region and a mammalian c-Fos first intron sequence.

5. The expression vector of claim 4, wherein the mammalian c-fos regulatory sequence is a rodent c-fos regulatory sequence comprising a rodent c-Fos 5′-non-coding region and a rodent c-Fos first intron sequence.

6. The expression vector of claim 5, wherein the rodent c-fos regulatory sequence is a mouse c-fos regulatory sequence comprising a mouse c-Fos 5′-non-coding region and a mouse c-Fos first intron sequence.

7. The expression vector of any of claims 1-6, wherein the expression cassette further comprises a sequence encoding a PEST peptide operably linked to the 3′ end of the polypeptide coding sequence.

8. The expression vector of any of claims 1-7, wherein the polypeptide coding sequence is heterologous to the c-fos regulatory sequence.

9. The expression vector of any of claims 1-8, wherein the polypeptide coding sequence encodes a light-responsive polypeptide.

10. The expression vector of claim 9, wherein the light-responsive polypeptide is a depolarizing opsin or a hyperpolarizing opsin.

11. The expression vector of any of claims 1-8, wherein the polypeptide coding sequence encodes a molecular tag.

12. The expression vector of any of claims 1-8, wherein the polypeptide coding sequence encodes a calcium sensor or voltage sensor or ion channel.

13. The expression vector of any of claims 1-8, wherein the polypeptide coding sequence encodes a toxic protein.

14. The expression vector of any of claims 1-8, wherein the polypeptide coding sequence encodes a receptor.

15. The expression vector of any of claims 1-8, wherein the polypeptide coding sequence encodes a nuclease.

16. The expression vector of any of claims 1-8, wherein the polypeptide coding sequence encodes a transcription factor.

17. The expression vector of any of claims 1-16, wherein the polypeptide coding sequence encodes a fusion protein comprising two or more polypeptides selected from the group consisting of: a light-responsive polypeptide, a molecular tag, a calcium sensor or voltage sensor or ion channel, a toxic protein, a receptor, a nuclease and a transcription factor.

18. The expression vector of any of claims 1-17, wherein the c-Fos 5′-non-coding region is less than 800 nucleotides in length.

19. The expression vector of claim 18, wherein the c-Fos 5′-non-coding region has a sequence identity of 80% or greater with SEQ ID NO:1.

20. The expression vector of any of claims 1-19, wherein the c-Fos first intron sequence comprises the entire first intron of a c-Fos gene or a degenerate sequence thereof.

21. The expression vector of any of claims 1-20, wherein the c-Fos first intron has a sequence identity of 80% or greater with SEQ ID NO:2.

22. The expression vector of any of claims 1-21, wherein the expression cassette further comprises a sequence of 50 to 200 nucleotides length positioned between the c-Fos 5′-non-coding region and the c-Fos first intron sequence.

23. The expression vector of claim 22, wherein the sequence of 50 to 200 nucleotides length comprises a sequence encoding the first exon of a c-Fos gene or a portion thereof.

24. The expression vector of claim 23, wherein the sequence encoding the first exon of a c-Fos gene has a sequence identity of 80% or greater with SEQ ID NO:3.

25. A recombinant adeno-associated virus (AAV), comprising an expression vector according to any of claims 1-24.

26. A method for activity-dependent labeling of an active cell, the method comprising:
(a) contacting a cell with an expression vector comprising an expression cassette comprising:
(i) a regulatory sequence comprising a c-Fos 5′-non-coding region and a c-Fos first intron sequence; and
(ii) a coding sequence encoding a labeling polypeptide operably linked to the regulatory sequence; and
(b) maintaining the cell under conditions permissive for activity-dependent activation of the regulatory sequence, wherein upon activity-dependent activation of the regulatory sequence the labeling polypeptide is expressed labeling the active cell.

27. The method of claim 26, wherein the contacting is performed in vitro.

28. The method of claim 26, wherein the contacting is performed in vivo.

29. The method according to any of claims 26-28, wherein the cell is a neuron.

30. The method according to claim 29, wherein the neuron is a mammalian neuron.

31. The method according to any of claims 29-30, wherein the neuron is present in the central nervous system of a vertebrate.

32. The method according to any of claims 26-31, wherein during the maintaining the cell is contacted with a stimulus thereby activating the regulatory sequence.

33. The method according to claim 32, wherein the stimulus is an electrical stimulus.

34. The method according to claim 32, wherein the stimulus is a pharmacological stimulus.

35. The method according to any of claims 26-34, wherein the contacting is performed in vivo by administering the expression vector to the central nervous system of a vertebrate and the maintaining comprises subjecting the vertebrate to a behavioral task sufficient to activate the regulatory sequence.

36. The method according to any of claims 26-35, wherein the labeling polypeptide is a molecular tag.

37. The method according to any of claims 26-36, wherein the labeling polypeptide is a recombinase and the cell comprises a recombination sequence that, upon recombination, induces expression of a molecular tag.

38. A method for activity-dependent control of an activated cell, the method comprising:
(a) contacting a cell with an expression vector comprising an expression cassette comprising:
(i) a regulatory sequence comprising a c-Fos 5′-non-coding region and a c-Fos first intron sequence; and
(ii) a coding sequence encoding a light-responsive polypeptide operably linked to the regulatory sequence;
(b) maintaining the cell under conditions permissive for activity-dependent activation of the regulatory sequence, wherein upon activity-dependent activation of the regulatory sequence the light-responsive polypeptide is expressed in the activated cell; and
(c) exposing the activated cell to light sufficient to trigger the light-responsive polypeptide to induce a response in the cell thereby controlling the activated cell.

39. The method of claim 38, wherein the contacting is performed in vitro.

40. The method of claim 39, wherein the contacting is performed in vivo.

41. The method according to any of claims 38-40, wherein the cell is a neuron.

42. The method according to claim 41, wherein the neuron is a mammalian neuron.

43. The method according to any of claims 38-42, wherein the neuron is present in the central nervous system of a vertebrate.

44. The method according to any of claims 38-43, wherein during the maintaining the cell is contacted with a stimulus thereby activating the regulatory sequence.

45. The method according to claim 44, wherein the stimulus is an electrical stimulus.

46. The method according to claim 44, wherein the stimulus is a pharmacological stimulus.

47. The method according to any of claims 38-46, wherein the contacting is performed in vivo by administering the expression vector to the central nervous system of a vertebrate and the maintaining comprises subjecting the vertebrate to a behavioral task sufficient to activate the regulatory sequence.

48. The method according to any of claims 38-47, wherein the response is depolarization.

49. The method according to any of claims 38-47, wherein the response is hyperpolarization.


SEQUENCE LISTING (64 sequences)

SEQ ID NO: 1 - DNA, 761 bp, Mus musculus
aagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgacc

SEQ ID NO: 2 - DNA, 754 bp, Mus musculus
gtgagtttggctttgtgtagccgccaggtccgcgctgagggtcgccgtggaggagacactggggtgtgactcgcaggggcgggggggtcttcctttttcgctctggagggagactggcgcggtcagagcagccttagcctgggaacccaggacttgtctgagcgcgtgcacacttgtcatagtaagacttagtgaccccttcccgcgcggcaggtttattctgagtggcctgcctgcattcttctctcggccgacttgtttctgagatcagccggggccaacaagtctcgagcaaagagtcgctaactagagtttgggaggcggcaaaccgcggcaatcccccctcccggggcagcctggagcagggaggagggaggagggaggagggtgctgcgggcgggtgtgtaaggcagtttcattgataaaaagcgagttcattctggagactccggagcagcgcctgcgtcagcgcagacgtcagggatatttataacaaaccccctttcgagcgagtgatgccgaagggataacgggaacgcagcagtaggatggaggagaaaggctgcgctgcggaattcaagggaggatattgggagagcttttatctccgatgaggtgcatacaggaagacataagcagtctctgaccggaatgcttctctctccctgcttcatgcgacactagggccacttgctccacctgtgtctggaacctcctcgctcacctccgctttcctctttttgttttgtttcag

SEQ ID NO: 3 - DNA, 141 bp, Mus musculus
atgatgttctcgggtttcaacgccgactacgaggcgtcatcctcccgctgcagtagcgcctccccggccggggacagcctttcctactaccattccccagccgactccttctccagcatgggctctcctgtcaacacacag

SEQ ID NO: 4 - DNA, 1500 bp, Mus musculus
cccagaggtgaccggcccagtcagtctaacccggcttgtcctctgcggaaggacaggaggccgagggcaagtaggggtgtgtttgttctacactgaagcacctgacctcttcaaagttccatcttccaagactcaaagctgttctcaggtcccagacgccaaaatctcggcacagctgggaacctttcttcccgtcccctctgcgcccccaccccccttcccaagtccgatctggaaaatcacccgctgcaggcgggttccttgtaagcgcagtttccaggctgcacgtattcagacccccatctccccagcaccgacttgctttctcctccccccccccccccgagctcacctcactttgtaattctgagctccccccctgccccgactcgccctctggtctcagctcaaaactaaacatacgaccccttcaggcatacttgtagggtggttttgcacaatgtttatccgtcagtgtcaacggggactgtcgccttgatagctctaagtggctaagggtcggggagtaggtgctgccgtcctttaaaacacgaatttatgaatgaacccagtactgtagttaaatcaggttattgtacacttatttacaatccttcacttgctgcttccaacctcagtcctaaagtttctccaggcaaggagctggagagaggggctgagaagctgaccccccctttttcttctctgcactgatttgggatggggggctgatgtgggcaagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgacc

SEQ ID NO: 5 - DNA, 767 bp, Mus musculus
gtgggcaagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgacc

SEQ ID NO: 6 - DNA, 60 bp, Mus musculus
tccattcacagcgcttctataaaggcgccagctgaggcgcctactactccaaccgcgact

SEQ ID NO: 7 - DNA, 1659 bp, Mus musculus
aagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggtgagtttggctttgtgtagccgccaggtccgcgctgagggtcgccgtggaggagacactggggtgtgactcgcaggggcgggggggtcttcctttttcgctctggagggagactggcgcggtcagagcagccttagcctgggaacccaggacttgtctgagcgcgtgcacacttgtcatagtaagacttagtgaccccttcccgcgcggcaggtttattctgagtggcctgcctgcattcttctctcggccgacttgtttctgagatcagccggggccaacaagtctcgagcaaagagtcgctaactagagtttgggaggcggcaaaccgcggcaatcccccctcccggggcagcctggagcagggaggagggaggagggaggagggtgctgcgggcgggtgtgtaaggcagtttcattgataaaaagcgagttcattctggagactccggagcagcgcctgcgtcagcgcagacgtcagggatatttataacaaaccccctttcgagcgagtgatgccgaagggataacgggaacgcagcagtaggatggaggagaaaggctgcgctgcggaattcaagggaggatattgggagagcttttatctccgatgaggtgcatacaggaagacataagcagtctctgaccggaatgcttctctctccctgcttcatgcgacactagggccacttgctccacctgtgtctggaacctcctcgctcacctccgctttcctctttttgttttgtttcagtaa

SEQ ID NO: 8 - DNA, 1500 bp, Homo sapiens
tgagccccggcagcgtgaccccggctgtcctacgcagcagggcaggagattggggggcgtggcacactctggagcaccttgcctccccaaagccccgtgttccaggacgtggagccgctcctggggtcccagcagtcgaggtattccgcccaggcgcagctggacactgtccttccagcccccgtcctccaccctccaagtccgcgctggaaaatcacccgctgcgggctcccgtaagcacagcttcctggcgggaccgaaccagccctcagcgcagatttgagttccccgcaggaagcacaccccgccttgtcatcccgaactgaccaccctgcccacataaccacacctcgcactccctacccctggggcccagctcagaaccgggcagacacccccttcaaatgtcttcgcacgtaggttttgcacagtgtttatctgctggtgtctcagggatttgacagtttccttaatattcccacacatggccgagaaaaataaataaataaatgcgctgtcttctttaaaaaaataaataaataaagtacccagtatcgtaaagtaggttatcgtattctcttattttggatcctccactttctgcttccaaacgcaggaacagtgctagtattgctcgagcccgagggctggaggttaggggatgaaggtctgcttccacgctttgcactgaattagggctagaattggggatgggggtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacg

SEQ ID NO: 9 - DNA, 784 bp, Homo sapiens
gtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacg

SEQ ID NO: 10 - DNA, 60 bp, Homo sapiens
ttcataaaacgcttgttataaaagcagtggctgcggcgcctcgtactccaaccgcatctg

SEQ ID NO: 11 - DNA, 753 bp, Homo sapiens
gtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttctccggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctag

SEQ ID NO: 12 - DNA, 141 bp, Homo sapiens
atgatgttctcgggcttcaacgcagactacgaggcgtcatcctcccgctgcagcagcgcgtccccggccggggatagcctctcttactaccactcacccgcagactccttctccagcatgggctcgcctgtcaacgcgcag

SEQ ID NO: 13 - DNA, 1678 bp, Homo sapiens
gtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttctccggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctag

SEQ ID NO: 14 - DNA, 1500 bp, Rattus norvegicus
ggagaagaggggacacatgagttctgcgaggatctgcggtttcctttcccagaggtgaccagcgctctggggccgagcccagtcagtctaacccggcttgtcctctgctgaaggacaggagactgagggcaagtaggggtgtgtttgttctacaccgaagcacccggcatctccaaagttccatcttccaagactcaaagctgtgctcaaagcagacgccaacatctctgcacagctgggaaccgtgcttccagtccgtcctcccctcctcccccatccccccctccccaagtccgaactggaaaatcacccgctgcgggttccttgtaagcgcagtttccaggctgcacggattcaggtccccacctcccctgtgcaccgaattgccttcttcccgggagctcacctcacttgtaattctgagcagacccctgccttcactcgccctctggcctccgctcaaaactgagcaaacgaccccttcaggcatccttgcagggtggttttgcacaatgtttatccgtcagtgtctcccgggacagtcaccctgattgttctaagtggccaagggtcggggagtgggtgctgtcgtcctttaaaacacgaatgtatgaatgaactcagtattgtaggtaaagcgggttattgaatacttacttagaatccttcacttactgcttccaacctcaggcctaatgttgcactgatttgggacggagagaggtctgatgtgggctagctttcctttgggaacagagacttggagcctttagggctgcgtgcctgcttctcctaataccagagacttttttaaaaagctccagattgctggacaatggaaaggagatgacccccagtctcatcccctgaccctgggaacagagtacacattgaatcaggtgcgaatgttcgctcgccttctctgcctttcccgcctcccctcccccggccgcggcccccgctccccccttgcgctgcaccctcagagttggctgcagccggcgagctgttcccgtcaatccctccctcctttacacaggatgtccatattaggacatctgcgtcagcaggtttccacggccggtccctgttgtcctggggggaaccatccccgaaatcctacatgcggagggtccaggagaccttctaagatcccaattgtgaacactcataggtgaaagttacagactgagacgggggttgagagcctggggcgtagagttgatgacagggagcccgcagagggcattcgggagcgctttcccccctccagtttctctgttccgctcatgacgtagtaagccattcaagcgcttctataaagcggccagctgaggcgcctactactccaaccgcgattgcagctagcaactgagaagactggatagagccggcggagccgcgaacgagcagtgaccgcgctcccacccagctctgctctgcagctcccaccagtgtctacccctggacccctcgccgagctttgcccaaaccacgacc

SEQ ID NO: 15 - DNA, 770 bp, Rattus norvegicus
gtgggctagctttcctttgggaacagagacttggagcctttagggctgcgtgcctgcttctcctaataccagagacttttttaaaaagctccagattgctggacaatggaaaggagatgacccccagtctcatcccctgaccctgggaacagagtacacattgaatcaggtgcgaatgttcgctcgccttctctgcctttcccgcctcccctcccccggccgcggcccccgctccccccttgcgctgcaccctcagagttggctgcagccggcgagctgttcccgtcaatccctccctcctttacacaggatgtccatattaggacatctgcgtcagcaggtttccacggccggtccctgttgtcctggggggaaccatccccgaaatcctacatgcggagggtccaggagaccttctaagatcccaattgtgaacactcataggtgaaagttacagactgagacgggggttgagagcctggggcgtagagttgatgacagggagcccgcagagggcattcgggagcgctttcccccctccagtttctctgttccgctcatgacgtagtaagccattcaagcgcttctataaagcggccagctgaggcgcctactactccaaccgcgattgcagctagcaactgagaagactggatagagccggcggagccgcgaacgagcagtgaccgcgctcccacccagctctgctctgcagctcccaccagtgtctacccctggacccctcgccgagctttgcccaaaccacgacc

SEQ ID NO: 16 - DNA, 760 bp, Rattus norvegicus
ggtgagtttggctttgtgcagtcgccaggtccgcgctgggggtcgccgaggagggcacattggggtgtgactgtcagggaagagtaggggtcttccttgtttgctccggagggagactggcgcggtcagagcagccctagcctgggaacccaggacttgtctgagcgcgtgcacacttgtcatactaagacttagtgacccccctcccgcgcggcaggtttactctgagtgtcctgcgctcttctctcggtgacttgtttctgagatcagccggggccaacaagtctctagcaaagactcgctaactagagcctgggaggcggcaaacggcggcaatcccccctcccggggcagcctggagcagggagaagggaggagggaggagggtgctgcgagccggtgtgtaaggcagtttcattgataaaaagcgagttcattctggagactccggagcagcgcctgcgtcagcgcagacgtcagggatatttataacaaaccccctttcgagcgagtgatgctgaagggataacgggaacgcagcagtaggatggaggagaaaggctgagctgcggaattcaggggaggatagaggatattgggagacctttttatctcggatgaagtgcatacaggaagacacaagcagtctctgaccagaatgcttctctctccctgcttcatgcgacactagggccacttgctccacctgtgtctggaacctcctcgctcacctccgctttcctctttttgttttgtttca

SEQ ID NO: 17 - DNA, 140 bp, Rattus norvegicus
atgatgttctcgggtttcaacgcggactacgaggcgtcatcctcccgctgcagtagcgcctccccggccggggacagcctttcctactaccattccccagccgactccttctccagcatgggctcccctgtcaacacaca

SEQ ID NO: 18 - DNA, 1670 bp, Rattus norvegicus
gtgggctagctttcctttgggaacagagacttggagcctttagggctgcgtgcctgcttctcctaataccagagacttttttaaaaagctccagattgctggacaatggaaaggagatgacccccagtctcatcccctgaccctgggaacagagtacacattgaatcaggtgcgaatgttcgctcgccttctctgcctttcccgcctcccctcccccggccgcggcccccgctccccccttgcgctgcaccctcagagttggctgcagccggcgagctgttcccgtcaatccctccctcctttacacaggatgtccatattaggacatctgcgtcagcaggtttccacggccggtccctgttgtcctggggggaaccatccccgaaatcctacatgcggagggtccaggagaccttctaagatcccaattgtgaacactcataggtgaaagttacagactgagacgggggttgagagcctggggcgtagagttgatgacagggagcccgcagagggcattcgggagcgctttcccccctccagtttctctgttccgctcatgacgtagtaagccattcaagcgcttctataaagcggccagctgaggcgcctactactccaaccgcgattgcagctagcaactgagaagactggatagagccggcggagccgcgaacgagcagtgaccgcgctcccacccagctctgctctgcagctcccaccagtgtctacccctggacccctcgccgagctttgcccaaaccacgaccatgatgttctcgggtttcaacgcggactacgaggcgtcatcctcccgctgcagtagcgcctccccggccggggacagcctttcctactaccattccccagccgactccttctccagcatgggctcccctgtcaacacacaggtgagtttggctttgtgcagtcgccaggtccgcgctgggggtcgccgaggagggcacattggggtgtgactgtcagggaagagtaggggtcttccttgtttgctccggagggagactggcgcggtcagagcagccctagcctgggaacccaggacttgtctgagcgcgtgcacacttgtcatactaagacttagtgacccccctcccgcgcggcaggtttactctgagtgtcctgcgctcttctctcggtgacttgtttctgagatcagccggggccaacaagtctctagcaaagactcgctaactagagcctgggaggcggcaaacggcggcaatcccccctcccggggcagcctggagcagggagaagggaggagggaggagggtgctgcgagccggtgtgtaaggcagtttcattgataaaaagcgagttcattctggagactccggagcagcgcctgcgtcagcgcagacgtcagggatatttataacaaaccccctttcgagcgagtgatgctgaagggataacgggaacgcagcagtaggatggaggagaaaggctgagctgcggaattcaggggaggatagaggatattgggagacctttttatctcggatgaagtgcatacaggaagacacaagcagtctctgaccagaatgcttctctctccctgcttcatgcgacactagggccacttgctccacctgtgtctggaacctcctcgctcacctccgctttcctctttttgttttgtttca

SEQ ID NO: 19 - protein, 380 aa, Mus musculus
MMFSGFNADYEASSSRCSSASPAGDSLSYYHSPADSFSSMGSPVNTQDFCADLSVSSANFIPTVTAISTSPDLQWLVQPTLVSSVAPSQTRAPHPYGLPTQSAGAYARAGMVKTVSGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEASTPESEEAFTLPLLNDPEPKPSLEPVKSISNVELKAEPFDDFLFPASSRPSGSETSRSVPDVDLSGSFYAADWEPLHSNSLGMGPMVTELEPLCTPVVTCTPGCTTYTSSFVFTYPEADSFPSCAAAHRKGSSSNEPSSDSLSSPTLLAL

SEQ ID NO: 20 - DNA, 2107 bp, Mus musculus
cagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccacccagtctgctggggcttacgccagagcgggaatggtgaagaccgtgtcaggaggcagagcgcagagcatcggcagaaggggcaaagtagagcagctatctcctgaagaggaagagaaacggagaatccgaagggaacggaataagatggctgcagccaagtgccggaatcggaggagggagctgacagatacactccaagcggagacagatcaacttgaagatgagaagtctgcgttgcagactgagattgccaatctgctgaaagagaaggaaaaactggagtttattttggcagcccaccgacctgcctgcaagatccccgatgaccttggcttcccagaggagatgtctgtggcctccctggatttgactggaggtctgcctgaggcttccaccccagagtctgaggaggccttcaccctgccccttctcaacgaccctgagcccaagccatccttggagccagtcaagagcatcagcaacgtggagctgaaggcagaaccctttgatgacttcttgtttccggcatcatctaggcccagtggctcagagacctcccgctctgtgccagatgtggacctgtccggttccttctatgcagcagactgggagcctctgcacagcaattccttggggatggggcccatggtcacagagctggagcccctgtgtactcccgtggtcacctgtactccgggctgcactacttacacgtcttcctttgtcttcacctaccctgaagctgactccttcccaagctgtgccgctgcccaccgaaagggcagcagcagcaacgagccctcctccgactccctgagctcacccacgctgctggccctgtgagcagtcagagaaggcaaggcagccggcatccagacgtgccactgcccgagctggtgcattacagagaggagaaacacgtcttccctcgaaggttcccgtcgacctagggaggaccttacctgttcgtgaaacacaccaggctgtgggcctcaaggacttgcaagcatccacatctggcctccagtcctcacctcttccagagatgtagcaaaaacaaaacaaaacaaaacaaaaaaccgcatggagtgtgttgttcctagtgacacctgagagctggtagttagtagagcatgtgagtcaaggcctggtctgtgtctcttttctctttctccttagttttctcatagcactaactaatctgttgggttcattattggaattaacctggtgctggattgtatctagtgcagctgattttaacaatacctactgtgttcctggcaatagcgtgttccaattagaaacgaccaatattaaactaagaaaagataggactttattttccagtagatagaaatcaatagctatatccatgtactgtagtccttcagcgtcaatgttcattgtcatgttactgatcatgcattgtcgaggtggtctgaatgttctgacattaacagttttccatgaaaacgtttttattgtgttttcaatttatttattaagatggattctcagatatttatatttttattttatttttttctaccctgaggtctttcgacatgtggaaagtgaatttgaatgaaaaattttaagcattgtttgcttattgttccaagacattgtcaataaaagcatttaagttgaaaaaaaaaaaaaaaa

SEQ ID NO: 21 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 22 - DNA, 2158 bp, Homo sapiens
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

SEQ ID NO: 23 - protein, 380 aa, Rattus norvegicus
MMFSGFNADYEASSSRCSSASPAGDSLSYYHSPADSFSSMGSPVNTQDFCADLSVSSANFIPTVTAISTSPDLQWLVQPTLVSSVAPSQTRAPHPYGLPTPSTGAYARAGVVKTMSGGRAQSIGRRGKVEQLSPEEEEKRRIRRERNKMAAAKCRNRRRELTDTLQAETDQLEDEKSALQTEIANLLKEKEKLEFILAAHRPACKIPNDLGFPEEMSVTSLDLTGGLPEATTPESEEAFTLPLLNDPEPKPSLEPVKNISNMELKAEPFDDFLFPASSRPSGSETARSVPDVDLSGSFYAADWEPLHSSSLGMGPMVTELEPLCTPVVTCTPSCTTYTSSFVFTYPEADSFPSCAAAHRKGSSSNEPSSDSLSSPTLLAL

SEQ ID NO: 24 - DNA, 1589 bp, Rattus norvegicus
ccaaccgcgattgcagctagcaactgagaagactggatagagccggcggagccgcgaacgagcagtgaccgcgctcccacccagctctgctctgcagctcccaccagtgtctacccctggacccctcgccgagctttgcccaaaccacgaccatgatgttctcgggtttcaacgcggactacgaggcgtcatcctcccgctgcagtagcgcctccccggccggggacagcctttcctactaccattccccagccgactccttctccagcatgggctcccctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccaccccgtcgaccggggcttacgccagagcgggagtggtgaagaccatgtcaggcggcagagcgcagagcatcggcagaaggggcaaagtagagcagctatctcctgaagaggaagagaaacggagaatccgaagggaaaggaataagatggctgcagccaagtgccggaatcggaggagggagctgacagatacgctccaagcggagacagatcaacttgaagacgagaagtctgcgttgcagaccgagattgccaatctactgaaagagaaggaaaaactggagtttattttggcagcccaccgacctgcctgcaagatccccaatgacctgggcttcccagaggagatgtctgtgacctccctggacttgactgggggtctgcctgaggctaccaccccagagtctgaggaggccttcaccctgcctcttctcaatgaccctgagcccaagccatccttggagccggtcaagaacattagcaacatggagctgaaggctgaaccctttgatgacttcttgtttccggcatcatctaggcccagtggctcggagactgcccgctctgtgccagatgtggacctgtctggttccttctatgcagcagactgggagcctctgcacagcagttccctggggatggggcccatggtcacagagctggagcccctgtgcactcccgttgtcacctgcactcccagctgcactacctatacgtcttcctttgtcttcacctaccccgaggctgactccttccctagctgcgcagctgcccaccgaaagggcagcagcagcaacgagccctcctctgactcactgagctcgcccacactgctagccctgtgagcagtcagagaaggcagggcagccggcactgactgagctggtgcattacagagagaagaaacaagtcttccctcgaggggttcccgtagacctagggaggaccttatctgtgcgtgaaacacaccaggctgtggacctcaaggacttgaaagcatccacatctggactccagtcctcacctcttccggagatgtagcaaaaaaacaaaaaaacaaaacaaaaaaaaaacaaaacaaaaaatcaaaagcaaccgcatggagtgtattgtttgtagtgacacctgagagctggtag

SEQ ID NO: 25 - protein, 1368 aa, Streptococcus pyogenes
MDKKYSIGLDIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKVDLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - protein, 343 aa, Artificial sequence
  synthetic amino acid sequence
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 27 - protein, 595 aa, Homo sapiens [sequence omitted]

SEQ ID NO: 28 - DNA, 120 bp, Artificial sequence
  synthetic polynucleotide sequence
agccatggcttcccgccggaggtggaggagcaggatgatggcacgctgcccatgtcttgtgcccaggagagcgggatggaccgtcaccctgcagcctgtgcttctgctaggatcaatgtg

SEQ ID NO: 29 - DNA, 2477 bp, Artificial sequence
  synthetic polynucleotide sequence
aagctttcctttaggaacagaggcttcgagcctttaaggctgcgtacttgcttctcctaataccagagactcaaaaaaaaaaaaaaagttccagattgctggacaatgacccgggtctcatcccttgaccctgggaaccgggtccacattgaatcaggtgcgaatgttcgctcgccttctctgcctttcccgcctcccctcccccggccgcggccccggttccccccctgcgctgcaccctcagagttggctgcagccggcgagctgttcccgtcaatccctccctcctttacacaggatgtccatattaggacatctgcgtcagcaggtttccacggccggtccctgttgttctgggggggggaccatctccgaaatcctacacgcggaaggtctaggagaccccctaagatcccaaatgtgaacactcataggtgaaagatgtatgccaagacgggggttgaaagcctggggcgtagagttgacgacagagcgcccgcagagggccttggggcgcgcttccccccccttccagttccgcccagtgacgtaggaagtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctcccaccagtgtctacccctggaccccttgccgggctttccccaaacttcgaccatgatgttctcgggtttcaacgccgactacgaggcgtcatcctcccgctgcagtagcgcctccccggccggggacagcctttcctactaccattccccagccgactccttctccagcatgggctctcctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccacccagtctgctggggcttacgccagagcgggaatggtgaagaccgtgtcaggaggcagagcgcagagcatcggcagaaggggcaaagtagagcagctatctcctgaagaggaagagaaacggagaatccgaagggaacggaataagatggctgcagccaagtgccggaatcggaggagggagctgacagatacactccaagcggagacagatcaacttgaagatgagaagtctgcgttgcagactgagattgccaatctgctgaaagagaaggaaaaactggagtttattttggcagcccaccgacctgcctgcaagatccccgatgaccttggcttcccagaggagatgtctgtggcctccctggatttgactggaggtctgcctgaggcttccaccccagagtctgaggaggccttcaccctgccccttctcaacgaccctgagcccaagccatccttggagccagtcaagagcatcagcaacgtggagctgaaggcagaaccctttgatgacttcttgtttccggcatcatctaggcccagtggctcagagacctcccgctctgtgccagatgtggacctgtccggttccttctatgcagcagactgggagcctctgcacagcaattccttggggatggggcccatggtcacagagctggagcccctgtgtactcccgtggtcacctgtactccgggctgcactacttacacgtcttcctttgtcttcacctaccctgaagctgactccttcccaagctgtgccgctgcccaccgaaagggcagcagcagcaacgagccctcctccgactccctgagctcacccacgctgctggccctgtgacccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 30 - protein, 258 aa, Halorubrum sodomense
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAAD

SEQ ID NO: 31 - protein, 531 aa, Artificial sequence
  synthetic amino acid sequence
MDPIALQAGYDLLGDGRPETLWLGIGTLLMLIGTFYFLVRGWGVTDKDAREYYAVTILVPGIASAAYLSMFFGIGLTEVTVGGEMLDIYYARYADWLFTTPLLLLDLALLAKVDRVTIGTLVGVDALMIVTGLIGALSHTAIARYSWWLFSTICMIVVLYFLATSLRSAAKERGPEVASTFNTLTALVLVLWTAYPILWIIGTEGAGVVGLGIETLLFMVLDVTAKVGFGFILLRSRAILGDTEAPEPSAGADVSAADRPVVAAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 32 - protein, 248 aa, Halorubrum sp.TP009
MDPIALQAGYDLLGDGRPETLWLGIGTLLMLIGTFYFIVKGWGVTDKEAREYYSITILVPGIASAAYLSMFFGIGLTEVTVAGEVLDIYYARYADWLFTTPLLLLDLALLAKVDRVSIGTLVGVDALMIVTGLIGALSHTPLARYSWWLFSTICMIVVLYFLATSLRAAAKERGPEVASTFNTLTALVLVLWTAYPILWIIGTEGAGVVGLGIETLLFMVLDVTAKVGFGFILLRSRAILGDTEAPEP

SEQ ID NO: 33 - protein, 223 aa, Guillardia theta
ASSFGKALLEFVFIVFACITLLLGINAAKSKAASRVLFPATFVTGIASIAYFSMASGGGWVIAPDCRQLFVARYLDWLITTPLLLIDLGLVAGVSRWDIMALCLSDVLMIATGAFGSLTVGNVKWVWWFFGMCWFLHIIFALGKSWAEAAKAKGGDSASVYSKIAGITVITWFCYPVVWVFAEGFGNFSVTFEVLIYGVLDVISKAVFGLILMSGAATGYESI

SEQ ID NO: 34 - protein, 262 aa, Oxyrrhis marina
MAPLAQDWTYAEWSAVYNALSFGIAGMGSATIFFWLQLPNVTKNYRTALTITGIVTLIATYHYFRIFNSWVAAFNVGLGVNGAYEVTVSGTPFNDAYRYVDWLLTVPLLLVELILVMKLPAKETVCLAWTLGIASAVMVALGYPGEIQDDLSVRWFWWACAMVPFVYVVGTLVVGLGAATAKQPEGVVDLVSAARYLTVVSWLTYPFVYIVKNIGLAGSTATMYEQIGYSAADVTAKAVFGVLIWAIANAKSRLEEEGKLRA

SEQ ID NO: 35 - protein, 313 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGA

SEQ ID NO: 36 - protein, 589 aa, Artificial sequence
  synthetic amino acid sequence
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDVELNGFWANGLNREGAIRIGEDDGARPVVAVSKAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 37 - protein, 310 aa, Chlamydomonas rheinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 38 - protein, 310 aa, Artificial sequence
  synthetic amino acid sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 39 - protein, 310 aa, Artificial sequence
  synthetic amino acid sequence
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTSPVILIHLSNLTGLSNDYSRRTMGLLVSAIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 40 - protein, 344 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 41 - protein, 344 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYAEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 42 - protein, 344 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 43 - protein, 344 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWETIYVATIEMIKFIIEYFHEFDEPAVIYSSNGNKTVWLRYATWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 44 - protein, 365 aa, Dunaliella salina
MRRRESQLAYLCLFVLIAGWAPRLTESAPDLAERRPPSERNTPYANIKKVPNITEPNANVQLDGWALYQDFYYLAGSDKEWVVGPSDQCYCRAWSKSHGTDREGEAAVVWAYIVFAICIVQLVYFMFAAWKATVGWEEVYVNIIELVHIALVIWVEFDKPAMLYLNDGQMVPWLRYSAWLLSCPVILIHLSNLTGLKGDYSKRTMGLLVSDIGTIVFGTSAALAPPNHVKVILFTIGLLYGLFTFFTAAKVYIEAYHTVPKGQCRNLVRAMAWTYFVSWAMFPILFILGREGFGHITYFGSSIGHFILEIFSKNLWSLLGHGLRYRIRQHIIIHGNLTKKNKINIAGDNVEVEEYVDSNDKDSDV

SEQ ID NO: 45 - protein, 273 aa, Natromonas pharaonis
VTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 46 - protein, 559 aa, Artificial sequence
  synthetic amino acid sequence
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 47 - protein, 542 aa, Artificial sequence
  synthetic amino acid sequence
MVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADDAAAKSRITSEGEYIPLDQIDINVVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKFCYENEV

SEQ ID NO: 48 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 49 - protein, 300 aa, Artificial sequence
  synthetic amino acid sequence
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTSPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 50 - protein, 300 aa, Artificial sequence
  synthetic amino acid sequence
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSAVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 51 - protein, 348 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 52 - protein, 348 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 53 - protein, 348 aa, Artificial sequence
  synthetic amino acid sequence
  misc_feature (167)..(167): Xaa can be any naturally occurring amino acid
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTXPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 54 - protein, 309 aa, Artificial sequence
  synthetic amino acid sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 55 - protein, 350 aa, Artificial sequence
  synthetic amino acid sequence
MVSRRPWLLALALAVALAAGSAGASTGSDATVPVATQDGPDYVFHRAHERMLFQTSYTLENNGSVICIPNNGQCFCLAWLKSNGTNAEKLAANILQWVTFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESS

SEQ ID NO: 56 - protein, 310 aa, Artificial sequence
  synthetic amino acid sequence
MDYGGALSAVGRELLFVTNPVVVNGSVLVPEDQCYCAGWIESRGTNGAQTASNVLQWLSAGFSILLLMFYAYQTWKSTCGWEEIYVCAISMVKVILEFFFSFKNPSMLYLATGHRVKWLRYASWLLTCPVILIRLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAVP

SEQ ID NO: 57 - protein, 344 aa, Artificial sequence
  synthetic amino acid sequence
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 58 - protein, 305 aa, Artificial sequence
  synthetic amino acid sequence
MDYGGALSAVGLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVLLIRLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISKYGSNIGHSILDLIAKQMWGVLGNYLRVKIHEHILLYGDIRKKQKITIAGQEMEVETLVAEEED

SEQ ID NO: 59 - protein, 350 aa, Artificial sequence
  synthetic amino acid sequence
MVSRRPWLLALALAVALAAGSAGASTGSDATVPVATQDGPDYVFHRAHERMLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 60 - protein, 310 aa, Artificial sequence
  synthetic amino acid sequence
MDYGGALSAVGLFQTSYTLENNGSVICIPNNGQCFCLAWLKSNGTNAEKLAANILQWVSFALSVACLGWYAYQAWRATCGWEEVYVALISMMKSIIEAFHSFDSPATLWLSSGNGVKWMRYGSWLLTCPVILIRLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISKYGSNIGHSILDLIAKQMWGVLGNYLRVKIHEHILLYGDIRKKQKITIAGQEMEVETLVAEEEDKYESS

SEQ ID NO: 61 - protein, 316 aa, Scherffelia dubia
MGGAPAPDAHSAPPGNDSAGGSEYHAPAGYQVNPPYHPVHGYEEQCSSIYIYYGALWEQETARGFQWFAVFLSALFLAFYGWHAYKASVGWEEVYVCSVELIKVILEIYFEFTSPAMLFLYGGNITPWLRYAEWLLTCPVILIHLSNITGLSEEYNKRTMALLVSDLGTICMGVTAALATGWVKWLFYCIGLVYGTQTFYNAGIIYVESYYIMPAGGCKKLVLAMTAVYYSSWLMFPGLFIFGPEGMHTLSVAGSTIGHTIADLLSKNIWGLLGHFLRIKIHEHIIMYGDIRRPVSSQFLGRKVDVLAFVTEEDKV

SEQ ID NO: 62 - protein, 350 aa, Chlamydomonas noctigama
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 63 - protein, 345 aa, Artificial sequence
  synthetic amino acid sequence
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 64 - protein, 325 aa, Stigeoclonium helveticum
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV